(19) United States
(12) Patent Application Publication
(10) Pub. No.: US 2003/0215903 A1
(43) Pub. Date: Nov. 20, 2003

(54) Nanostructures containing PNA joining or functional elements

(76) Inventors: Paul L. Hyman, Newburyport, MA (US); Edward B. Goldberg, Newton, MA (US)

Correspondence Address: OPPEDAHL AND LARSON LLP, P O BOX 5068, DILLON, CO, 80435-5068, US

(21) Appl. No.: 10/370,685

(22) Filed: Feb. 21, 2003

Related U.S. Application Data
(63) Continuation-in-part of application No. 10/080,608, filed on Feb. 21, 2002.

(51) Int. Cl. C12P 21/06; C07K 14/00

(52) U.S. Cl. 435/68.1; 530/350

(57) ABSTRACT

Nanostructures are made that include at least one species of assembly unit comprising a peptide nucleic acid (PNA). PNA assembly units may have one or two PNA joining elements. In addition, the PNA assembly units may contain structural elements, and/or other functional or joining elements. The nanostructure is suitably prepared using a staged assembly method. In this method, a nanostructure intermediate having at least one unbound joining element is contacted with an assembly unit having a plurality of different joining elements. None of the joining elements of the assembly unit can interact with itself or with another joining element of the same assembly unit. However, one of the joining elements of the assembly unit can interact with the unbound joining element of the nanostructure intermediate, so that the assembly unit is non-covalently bound to the nanostructure intermediate to form a new nanostructure intermediate for use in subsequent cycles. Unbound assembly units are removed and the cycles is repeated for a sufficient number of cycles to form a nanostructure. In one specific method, the complementary joining elements in at least one cycle are PNA joining elements.

This application is a continuation-in-part of U.S. patent application Ser. No. 10/080,608, filed Feb. 21, 2002, which is incorporated herein by reference.

### TECHNICAL FIELD

The present invention relates to methods for the assembly of nanostructures containing peptide nucleic acids (PNAs), to PNA assembly units for use in the construction of such nanostructures, and to nanostructures containing PNA assembly units.

### BACKGROUND OF THE INVENTION

Nanostructures are structures with individual components having one or more characteristic dimensions in the nanometer range (from about 1-100 nm). The advantages of assembling structures in which components have physical dimensions in the nanometer range have been discussed and speculated upon by scientists for over forty years. The advantages of these structures were first pointed out by Feynman (1959, There's Plenty of Room at the Bottom, An Invitation to Enter a New Field of Physics (lecture), Dec. 29, 1959, American Physical Society, California Institute of Technology, reprinted in Engineering and Science, February 1960, California Institute of Technology, Pasadena, Calif.) and greatly expanded on by Drexler (1986, Engines of Creation, Garden City, N.Y.: Anchor Press/Doubleday). These scientists envisioned enormous utility in the creation of architectures with very small characteristic dimensions. The potential applications of nanotechnology are pervasive and the expected impact on society is huge (e.g., 2000, Nanotechnology Research Directions: IWGN Workshop Report; Vision for Nanotechnology R & D in the Next Decade; eds. M. C. Roco, R. S. Williams and P. Alivisatos, Kluwer Academic Publishers). It is predicted that there will be a vast number of potential applications for nanoscale devices and structures including electronic and photonic components; medical sensors; novel materials; biocompatible devices; nanoelectronics and nanocircuits; and computer technology.

The physical and chemical attributes of a nanostructure depend on the building blocks from which it is made. For example, the size of these building blocks, and the angles at which they join plays an important role in determining the properties of the nanostructure, and the positions in which functional elements can be placed. The art provides numerous examples of different types of materials which can be used in nanostructures, including DNA (U.S. Pat. Nos. 5,468,851, 5,948,897 and 6,072,044; WO 01/00876), bacteriophage T-even tail fibers (U.S. Pat. Nos. 5,864,013 and 5,877,279 and WO 00/77196), self-aligning peptides modeled on human elastin and other fibrous proteins (U.S. Pat. No. 5,969,106), and artificial peptide recognition sequences (U.S. Pat. No. 5,712,366). Nevertheless, there is a continuing need for additional types of building blocks to provide the diversity which may be required to meet all of the potential applications for nanostructures . The present application provides a further class of building blocks which can be used in homogeneous nanostructures containing building blocks of only this class, or in heterogeneous nanostructures in combination with building blocks of other classes.

### SUMMARY OF THE INVENTION

The present invention provides nanostructures formed from a plurality of species of assembly units. With some exceptions, such as capping units, these assembly units comprise a plurality of different joining elements. In the nanostructures of the invention, the nanostructure includes at least one species of assembly unit in which a t least one joining or functional element comprises a peptide nucleic acid. The PNA assembly units may have two PNA joining elements. In addition, the PNA assembly units may contain other structural, functional and joining elements.

The nanostructure of the invention is suitably prepared using a staged assembly method. In this method, a nanostructure intermediate comprising at least one unbound joining element is contacted with an assembly unit comprising a plurality of different joining elements, wherein:

(i) none of the joining elements of said plurality of different joining elements can interact with itself or with another joining element of said plurality, and

(ii) a single joining element of said plurality and a single unbound joining element of the nanostructure intermediate are complementary joining element.

As a result, the assembly unit is non-covalently bound to the nanostructure intermediate to form a new nanostructure intermediate for use in subsequent cycles. Unbound assembly units are then removed and the process is repeated for a sufficient number of cycles to form a nanostructure. In the method of the invention, the complementary joining elements in at least one cycle comprise a PNA.

### BRIEF DESCRIPTION OF THE DRAWINGS

FIG. 1. Comparison of PNA (peptide nucleic acid, left) and DNA (right) structure. Note that PNA has a neutral peptide or peptide-like backbone instead of a negatively-charged sugar-phosphate backbone.

FIGS. 2(A-B). Two PNA/oligopeptide units can dimerize to form a single assembly unit. Two possible configurations for an assembly unit are shown here (FIG. 19A and FIG. 19B). The PNA portion provides joining elements A and B′, while the oligopeptide portion forms two coiled coil structural elements (S) stabilized by disulfide bonds at either end. One or more functional units (F), comprised of, e.g., protein segments, may also be incorporated into the assembly unit. In certain embodiments, the assembly unit can have a randomly coiled peptide that comprises a functional element, F, in the internal or center portion of the dimer (FIG. 19A) or at the end of the PNA molecule opposite the end comprising the joining element (FIG. 19B). In each of these diagrams, the N-terminal end of the PNA/oligopeptide unit is towards the left of the diagram and the C-terminal end is towards the right.

FIG. 3. Line diagram indicating the order of elements of the upper synthetic protein monomer forming the staged assembly subunit shown in FIG. 19A. The order of the elements in the corresponding lower unit would be identical except that the PNA element is at the C-terminus. This reflects the parallel arrangement of the leucine zippers aligning the two units. The functionality sequence encodes the region at which a functional element may be added to the assembly subunit. Glycines separate each element to reduce steric interference between elements. Numbers below the line indicate the typical length in residues of each element.

FIG. 4. Diagram of ROP protein, a four-helix bundle.

FIG. 5. Staged assembly of assembly units. In practice, each step in the staged assembly will be carried out in a massively parallel fashion. In step 1, an initiator unit is immobilized on a solid substrate. In the embodiment of the invention illustrated here, the initiator unit has a single joining element. In step 2, a second assembly unit is added. The second unit has two non-complementary joining elements, so that the units will not self-associate in solution. One of the joining elements on the second assembly unit is complementary to the joining element on the initiator unit. Unbound assembly units are washed away between each step (not shown).

After incubation, the second assembly unit binds to the initiator unit, resulting in the formation of a nanostructure intermediate made up of two assembly units. In step 3, a third assembly unit is added. This unit has two non-complementary joining elements, one of which is complementary to the only unpaired joining element on the nanostructure intermediate. This unit also has a functional unit (“F3”).

A fourth assembly unit with functional element “F4” and a fifth assembly unit with functional element “F5”are added in steps 4 and 5, respectively, in a manner exactly analogous to steps 2 and 3. In each case, the choice of joining elements prevents more than one unit from being added at a time, and leads to a tightly controlled assembly of functional units in pre-designated positions.

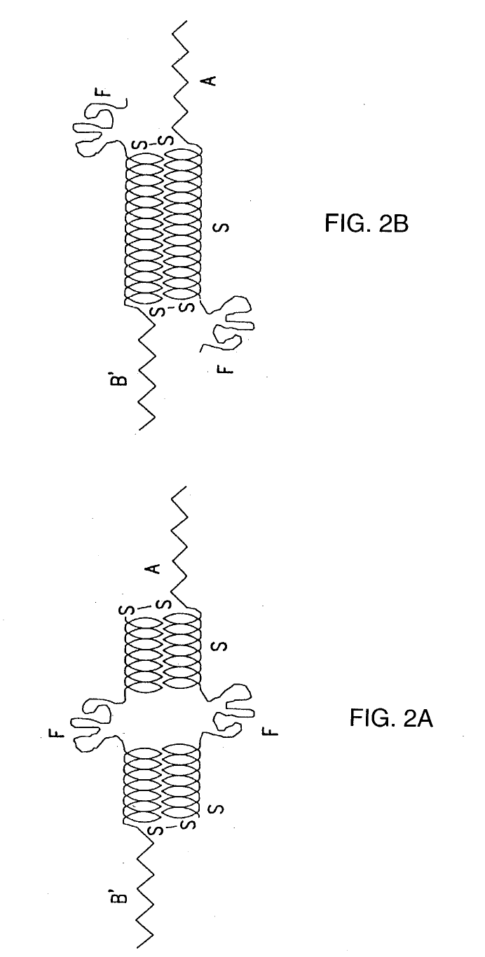

FIG. 6. Generation of a nanostructure from subassemblies. A nanostructure can be generated through the sequential addition of subassemblies, using steps analogous to those used for the addition of individual assembly units as illustrated above in FIG. 2. The arrow indicates the addition of a subassembly to a growing nanostructure.

FIG. 7. A diagram illustrating the addition of protein units and inorganic elements to a nanostructure according to the staged assembly methods of the invention. In step 1, an initiator unit is bound to a solid substrate. In step 2, an assembly unit is bound specifically to the initiator unit. In step 3, an additional assembly unit is bound to the nanostructure undergoing assembly. This assembly unit comprises an engineered binding site specific for a particular inorganic element. In step 4, the inorganic element (depicted as a cross-hatched oval) is added to the structure and bound by the engineered binding site. Step 5 adds another assembly unit with a binding site engineered for specificity to a second type of inorganic element, and that second inorganic element (depicted as a hatched diamond) is added in step 6.

FIG. 8. Diagram of eleven steps of a staged assembly that utilizes four bispecific assembly units and one tetraspecific assembly unit to make a two-dimensional nanostructure.

FIGS. 9(A-B). Diagram of a staged assembly that utilizes nanostructure intermediates as subassemblies. In Steps 1-3, a nanostructure intermediate is constructed, two joining elements are capped and the nanostructure intermediate is released from the solid substrate. In Step 5, the nanostructure intermediate from Step 3 is added to an assembly intermediate (shown in Step 4 attached to the solid substrate) as an intact subassembly.

### DETAILED DESCRIPTION OF THE INVENTION

Definitions:

The terms in this application are generally used in a manner consistent with their ordinary meaning in the art. To provide clarity, however, in the event of a disagreement in the art, the following definitions control.

Assembly Unit: An assembly unit is an assemblage of atoms and/or molecules comprising structural elements, joining elements and/or functional elements. Preferably, an assembly unit is added to a nanostructure as a single unit through the formation of specific, non-covalent interactions. An assembly unit may two or more sub-assembly units. An assembly unit may comprise one or more structural elements, and may further comprise one or more functional elements and one or more joining elements. If an assembly unit comprises a functional element, that functional element may be attached to or incorporated within a joining element or, in certain embodiments, a structural element. Such an assembly unit, which may comprise a structural element and one or a plurality of non-interacting joining elements, may be, in certain embodiments, structurally rigid and have well-defined recognition and binding properties.

Assembly Unit, Initiator: An initiator assembly unit is the first assembly unit incorporated into a nanostructure that is formed by the staged assembly method of the invention. It may be attached, by covalent or non-covalent interactions, to a solid substrate or other matrix as the first step in a staged assembly process. An initiator assembly unit is also known as an “initiator unit.”

Bottom-up: Bottom-up assembly of a structure (e.g., a nanostructure) is formation of the structure through the joining together of substructures using, for example, self-assembly or staged assembly.

Capping Unit: A capping unit is an assembly unit that comprises at most one joining element. Additional assembly units cannot be incorporated into the nanostructure through interactions with the capping unit once the capping unit has been incorporated into the nanostructure.

Functional Element: A functional element is a moiety exhibiting any desirable physical, chemical or biological property that may be built into, bound or placed by specific covalent or non-covalent interactions, at well-defined sites in a nanostructure. Alternatively, a functional element can be used to provide an attachment site for a moiety with a desirable physical, chemical, or biological property. Examples of functional elements include, without limitation, a peptide, protein (e.g., enzyme), protein domain, small molecule, inorganic nanoparticle, atom, cluster of atoms, magnetic, photonic or electronic nanoparticles, or a marker such as a radioactive molecule, chromophore, fluorophore, chemiluminescent molecule, or enzymatic marker. Such functional elements can also be used for cross-linking linear, one-dimensional nanostructures to form two-dimensional and three-dimensional nanostructures.

Joining Element: A joining element is a portion of an assembly unit that confers binding properties on the unit, including, but not limited to: binding domain, hapten, antigen, peptide, PNA, DNA, RNA, aptamer, polymer or other moiety, or combination thereof, that can interact through specific, non-covalent interactions, with another joining element.

Joining Elements, Complementary: Complementary joining elements are two joining elements that interact with one another through specific, non-covalent interactions.

Joining Elements, Non-Complementary: Non-complementary joining elements are two joining elements that do not specifically interact with one another, nor demonstrate any tendency to specifically interact with one another.

Joining Pair: A joining pair is two complementary joining elements.

Nanomaterial: A nanomaterial is a material made up of a crystalline, partially crystalline or non-crystalline assemblage of nanoparticles.

Nanoparticle: A nanoparticle is an assemblage of atoms or molecules, bound together to form a structure with dimensions in the nanometer range (1-1000 nm). The particle may be homogeneous or heterogeneous. Nanoparticles that contain a single crystal domain are also called nanocrystals.

Nanostructure or Nanodevice: A nanostructure or nanodevice is an assemblage of atoms and/or molecules comprising assembly units, i.e., structural, functional and/or joining elements, the elements having at least one characteristic length (dimension) in the nanometer range, in which the positions of the assembly units relative to each other are established in a defined geometry. The nanostructure or nanodevice may also have functional substitutents attached to it to provide specific functionality.

Nanostructure intermediate: A nanostructure intermediate is an intermediate substructure created during the assembly of a nanostructure to which additional assembly units can be added. In the final step, the intermediate and the nanostructure are the same.

Non-covalent Interaction, Specific: A specific non-covalent interaction is, for example, an interaction that occurs between an assembly unit and a nanostructure intermediate.

Protein: In this application, the term “protein” is used generically to referred to peptides, polypeptides and proteins comprising a plurality of amino acids, and is not intended to imply any minimum number of amino acids.

Removing: Removing of unbound assembly units is accomplished when they are rendered unable to participate in further reactions with the growing nanostructure, whether or not they are physically removed.

Self-assembly: Self-assembly is spontaneous organization of components into an ordered structure. Also known as auto-assembly.

Staged Assembly of a Nanostructure: Staged assembly of a nanostructure is a process for the assembly of a nanostructure wherein a series of assembly units are added in a pre-designated order, starting with an initiator unit that is typically immobilized on a solid matrix or substrate. Each step results in the creation of an intermediate substructure, referred to as the nanostructure intermediate, to which additional assembly units can then be added. An assembly step comprises (i) a linking step, wherein an assembly unit is linked to an initiator unit or nanostructure intermediate through the incubation of the matrix or substrate with attached initiator unit or nanostructure intermediate in a solution comprising the next assembly units to be added; and (ii) a removal step, e.g., a washing step, in which excess assembly units are removed from the proximity of the intermediate structure or completed nanostructure. Staged assembly continues by repeating steps (i) and (ii) until all of the assembly units are incorporated into the nanostructure according to the desired design of the nanostructure. Assembly units bind to the initiator unit or nanostructure intermediate through the formation of specific, non-covalent bonds. The joining elements of the assembly units are chosen so that they attach only at pre-designated sites on the nanostructure intermediate. The geometry of the assembly units, the structural elements, and the relative placement of joining elements and functional elements, and the sequence by which assembly units are added to the nanostructure are all designed so that functional units are placed at pre-designated positions relative to one another in the structure, thereby conferring a desired function on the completely assembled nanostructure.

Stringency: The extent to which experimental conditions impose a high degree of complementarity on two nucleic acid sequences to achieve a stable hybridization interaction. Highly or moderately stringent conditions are commonly known in the art. By way of example and not limitation, exemplary conditions of high stringency are as follows: Prehybridization of filters containing DNA is carried out for 8 h to overnight at 65° C. in buffer composed of 6× SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2× SSC, 0.01% PVP, 0.01% Ficoll, and 0.01% BSA. This is followed by a wash in 0.1× SSC at 50° C. for 45 min before autoradiography. Other conditions of high stringency that may be used are well known in the art. By way of further example and not limitation, exemplary conditions of moderate stringency are as follows: Filters containing DNA are pretreated for 6 h at 55° C. in a solution containing 6× SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 h at 55° C., and then washed twice for 30 minutes at 60° C. in a solution containing 1× SSC and 0.1% SDS. Filters are blotted dry and exposed for autoradiography. Other conditions of moderate stringency that may be used are well-known in the art. Other conditions of high stringency that may be used are, in general, for probes between 14 and 70 nucleotides in length the melting temperature (TM) is calculated using the formula: Tm(° C.)=81.5+16.6(log[monovalent cations (molar)])+0.41 (% G+C)−(500/N) where N is the length of the probe. If the hybridization is carried out in a solution containing formamide, the melting temperature is calculated using the equation Tm(° C.)=81.5+16.6(log[monovalent cations (molar)])+0.41(% G+C)−(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids).

Structural Element: A structural element is a portion of an assembly unit that provides a structural or geometric linkage between joining elements, thereby providing a geometric linkage between adjoining assembly units. Structural elements provide the structural framework for the nanostructure of which they are a part.

Subassembly: A subassembly is an assemblage of atoms or molecules consisting of multiple assembly units bound together and capable of being added as a whole to an assembly intermediate (e.g., a nanostructure intermediate). In many embodiments of the invention, structural elements also support the functional elements in the assembly unit.

Top-down: Top-down assembly of a structure (e.g., a nanostructure) is formation of a structure through the processing of a larger initial structure using, for example, lithographic techniques.

PNA Assembly Units

The present invention provides a new class of assembly units that can be used in production of nanostructures. These “PNA assembly units” contain at least one joining or functional element that is a PNA. In addition, the assembly unit may contain structural elements and/or other joining and functional elements.

PNA Joining Elements

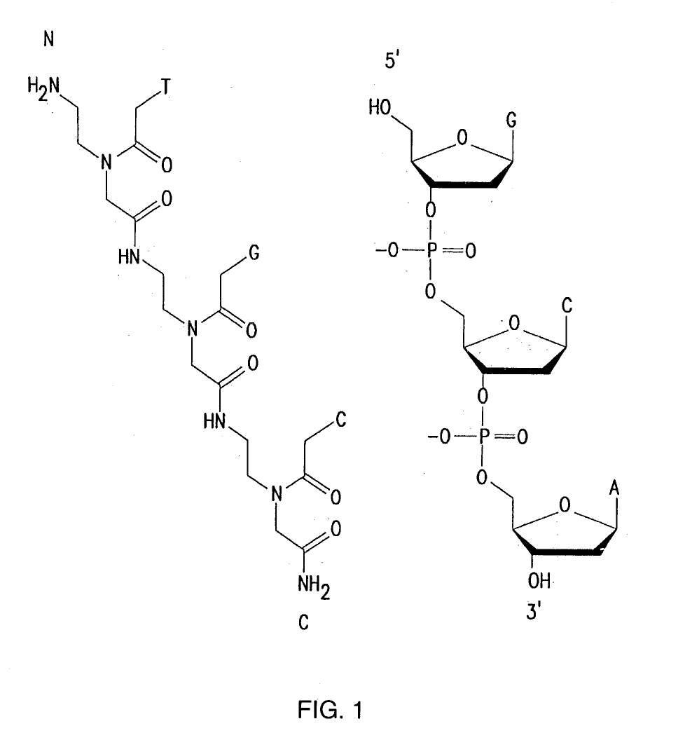

In certain embodiments of the invention, a joining element comprises a peptide nucleic acid (PNA) and may have any of a number of general forms, such as that shown in FIG. 1. PNA is a structural homologue of DNA that was first described by Nielsen et al. (1991, Sequence-selective recognition of DNA by strand displacement with a thymine-substituted polyamide, Science 254: 1497-1500) and has a neutral peptide or peptide-like backbone instead of a negatively-charged sugar-phosphate backbone (FIG. 1). Therefore, a PNA may be viewed as a protein or oligopeptide in which the amino acid side chains have been replaced with the pyrimidine and purine bases of DNA. The same nitrogenous bases (i.e. adenine, guanine, cytosine and thymine) are used in PNAs as are found in DNA and RNA; PNAs bind to DNA and RNA molecules according to Watson-Crick and/or Hoogsteen base pairing rules. PNAs are not generally recognized as substrates by DNA polymerases, nucleic acid binding proteins, or other enzymes, including proteases and nucleases, although some exceptions do exist (see, e.g., Lutz et al., 1997, Recognition of uncharged polyamide-linked nucleic acid analogs by DNA polymerases and reverse transcriptases, J. Am. Chem. Soc. 119: 3177-78). The biology of PNAs has been reviewed extensively (see, e.g., Nielsen et al., 1992, Peptide nucleic acids (PNA). DNA analogues with a polyamide backbone, In Antisense Research and Applications, Crooke and Lebleu, eds., CRC Press, pp. 363-72; Nielsen et al., 1993, Peptide nucleic acids (PNAs): potential antisense and anti-gene agents, Anticancer Drug Des. 8(1): 53-63; Buchardt et al., 1993, Peptide nucleic acids and their potential applications in biotechnology, Trends Biotechnol. 11(9):384-86; Nielsen et al., 1994, Peptide nucleic acid (PNA). A DNA mimic with a peptide backbone, Bioconjug. Chem. 5(1): 3-7; Nielsen et al., 1996, Peptide nucleic acid (PNA): A lead for gene therapeutic drugs, in Antisense Therapeutics Vol. 4, Trainor, ed., SECOM Science Publishers B. V., Leiden, pp. 76-84; Nielsen, 1995, DNA analogues with nonphosphodiester backbones, Ann. Rev. Biophys. Biomol. Struct. 24: 167-83; Hyrup and Nielsen, 1996, Peptide nucleic acids (PNA): synthesis, properties and potential applications, Bioorg. Med. Chem. 4:5-23; De Mesmaeker et al., 1995, Backbone modifications in oligonucleotides and peptide nucleic acid systems, Curr. Opin. Struct. Biol. 5: 343-55; Dueholm and Nielsen, 1997, Chemical aspects of peptide nucleic acid, New J. Chem. 21: 19-31; Knudsen and Nielsen, 1997, Application of PNA in cancer therapy, Anti-Cancer Drug 8: 113-18; Nielsen, 1997, Design of Sequence Specific DNA Binding Ligands, Chemistry 3: 505-08; Corey, 1997, Peptide nucleic acids: expanding the scope of nucleic acid recognition. Trends Biotechnol. 15(6):224-29; Nielsen and Ørum, 1995, Peptide nucleic acid (PNA) as new biomolecular tools, in Molecular Biology: Current Innovations and Future Trends, Part 2, (Griffin, H., Ed.), Horizon Scientific Press, UK, pp. 73-86; Nielsen and Haaima, 1997, Peptide Nucleic Acid (PNA). A DNA Mimic with a Pseudopeptide Backbone, Chem. Soc. Rev.: 73-78).

In PNA, as shown in FIG. 1, the phosphoribose backbone may be replaced, for example, by repeating units of N-(2-aminoethyl)-glycine linked by amide bonds (Egholm et al., 1992, Peptide nucleic acids (PNA), Oligonucleotide analogues with an achiral peptide backbone, J. Am. Chem. Soc. 114: 1895-97). Other substitutions in PNA of a neutral peptide or peptide-like backbone for a negatively-charged sugar-phosphate backbone are commonly known in the art and will be readily apparent to the skilled artisan. PNAs with modified polyamide backbones have been described, for example, in Hyrup et al. (1994, Structure-Activity studies of the binding modified Peptide Nucleic Acids, Journal of the American Chemical Society 116: 7964-70); Dueholm et al. (1994, Peptide Nucleic Acid (PNA) with a chiral backbone based on alanine, Bioorg. Med. Chem. Lett. 4: 1077-80); Peyman et al. (1996, Phosphonic Esters Nucleic Acids (PHONAs): Oligonucleotide Analogues with an Achiral Phosphonic Acid Ester Backbone, Angew. Chem. Int. Ed. Engl. 35: 2636-38); van der Laan et al. (1996, An approach towards the synthesis of oligomers containing a N-2-hydroxyethyl-aminomethylphosphonate backbone—A novel PNA analogue, Tetrahedron Letters 37: 7857-60); Jordan et al. (1997, Synthesis of new building blocks for peptide nucleic acids containing monomers with variations in the backbone, Bioorg. Med. Chem. Lett. 7: 681-86); Goodnow et al. (1997, Oligomer Synthesis and DNA/RNA Recognition Properties of a Novel Oligonucleotide Backbone Analog: Glucopyranosyl Nucleic Amide (GNA), Tetrahedron Lett. 38: 3199-3202); Zhang et al. (1999, Studies on the synthesis and properties of new PNA analogs consisting of L- and D-lysine backbones, Bioorg. Med. Chem. Lett. 9: 2903-08); Stammers et al. (1999, Synthesis of enantiomerically pure backbone alkyl substituted peptide nucleic acids utilizing the Et-DuPHOS-Rh+ hydrogenation of enamido esters, Tetrahedron Lett., 40, 3325-3328); Puschl et al. (2000, Pyrrolidine PNA: A Novel Conformationally Restricted PNA Analogue, Organic Letters 2: 4161-63); Vilaivan et al. (2000, Synthesis and properties of chiral peptide nucleic acids with a N-aminoethyl-D-proline backbone, Bioorg Med Chem Lett 10(22):2541-45); Yu et al., 2001, Synthesis and characterization of a tetranucleotide analogue containing alternating phosphonate-amide backbone linkages, Bioorg. Med. Chem. 9(1):107-19); Fader et al. (2001, Backbone modifications of aromatic peptide nucleic acid (APNA) monomers and their hybridization properties with DNA and RNA, J. Org. Chem. 66: 3372-79).

The nitrogenous bases of a PNA are attached to the neutral backbone by methylene carbonyl linkages. Because PNA does not have a highly-charged sugar-phosphate backbone, PNA binding to a target nucleic acid is stronger than with conventional nucleic acids, and that binding, once established, is virtually independent of salt concentration. This is reflected, quantitatively, by a high thermal stability of duplexes containing PNA.

Because the peptide backbone is uncharged, base-pairing between two complementary PNA molecules, or between, e.g., DNA and PNA in a DNA/PNA hybrid, is much stronger than in the corresponding DNA/DNA hybrid. Binding of a PNA to its complementary DNA or RNA target will occur more quickly than binding of the equivalent nucleic acid probe. The affinity of the PNA is so high that it can displace the corresponding strand in double stranded DNA (Nielsen et al., 1991, Sequence-selective recognition of DNA by strand displacement with a thymine substituted polyamide, Science 254: 1497-1500).

PNAs generally have a melting temperature that is higher than the corresponding DNA duplex, by approximately ⁻1° C. per base at moderate salt conditions (e.g., 100 mM NaCl) (Nielsen et al., 1991, Sequence-selective recognition of DNA by strand displacement with a thymine-substituted polyamide, Science 254: 1497-1500; Peffer et al., 1993, Strand-invasion of duplex DNA by peptide nucleic acid oligomers, Proc. Natl. Acad. Sci. USA 90: 10648-52; Demidov et al., 1995, Kinetics and mechanism of polyamide (“peptide”) nucleic acid binding to duplex DNA, Proc. Natl. Acad. Sci. USA 92: 2637-41). Thermal stability of a DNA-DNA duplex (as indicated by Tₘ) is approximated using an estimate of 2° C. per AT base pair and 4° C. per GC base pair, whereby a 10 bp DNA duplex with 50% GC content would be estimated to have melting temperature of about 30° C. Accordingly, the corresponding PNA therefore would have a melting temperature of about 40° C. Similarly an 18 residue PNA duplex (50% GC) would be estimated to have a melting temperature of about 72° C. Therefore, in certain embodiments of the present invention a PNA joining element has about 8 residues to about 20 residues, about 10 residues to about 18 residues, or about 12 residues to about 16 residues.

In other embodiments, PNAs having fewer residues can be designed that have higher melting temperatures by taking advantage of the PNA's ability to form triple helices. In a specific embodiment, three PNA strands (two polypyrimidine, one polypurine) form this extremely stable structure. The structure can be further stabilized by using two PNA's such that one has two polypyrimidine PNA stretches separated by a glycine spacer, wherein the glycine spacer generally comprises three to five glycine residues. When mixed with the corresponding polypurine PNA, the two polypyrimidine PNA segments fold around the glycine space to form this triple helix. Having the “two” polypyrimidine strands on the same molecule raises the effective concentration and hence the rate of formation and strength of the triplex helix. For a staged assembly joining pair, one joining element of the joining pair would contain the polypurine strand while the other joining element of the joining pair is a double-length polypyrimidine PNA joining element.

PNAs may be synthesized by methods well known in the art using chemistries similar to those used for synthesis of nucleic acids and peptides. PNA monomers used in such syntheses are hybrids of nucleosides and amino acids. PNA products, services (such as custom-synthesis of PNA molecules), and technical support are commercially available from PerSeptive Biosystems, Inc. (a division of Applied Biosystems, Foster City, Calif.). PNA may be synthesized using commercially available reagents and equipment or can be purchased from contract manufacturers such as PerSeptive Biosystems, Inc. PNA oligomers may also be manually synthesized using either Fmoc or t-Boc protected monomers using standard peptide chemistry protocols. Similarly, standard peptide purification conditions may be used to purify PNA following synthesis.

In certain embodiments, a PNA used in the methods of the invention is a chimeric PNA or a binding derivative or modified version thereof, and references to PNA should be understood to encompass both PNSs and these variations. A chimeric PNA is a molecule that is modified at the base moiety or the peptide backbone, and that may include other appending groups or labels. A chimeric PNA also may be a molecule that comprises a PNA sequence linked by a covalent bond(s) to one or more amino acids or to a sequence of two or more contiguous amino acids.

For example, a chimeric or modified PNA may comprise at least one modified base moiety which is selected from the group including but not limited to 5-fluorouracil; 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In a specific embodiment, a modified or chimeric PNA contains the “universal base” 3-nitropyrrole (Zhang et al., 2001, Peptide nucleic acid-DNA duplexes containing the universal base 3-nitropyrrole, Methods 23: 132-40).

Once a desired PNA is synthesized, it is cleaved from the solid support on which it was synthesized and treated, by methods known in the art, to remove any protecting groups present. The PNA may then be purified by any method known in the art, including extraction and gel purification. The concentration and purity of the PNA may be determined by examining PNA that has been separated on an acrylamide gel, or by measuring the optical density in a spectrophotometer.

In certain embodiments of the invention, a joining pair comprises a complementary pair of PNA joining elements that are capable of binding via standard Watson-Crick and/or Hoogsteen base-pairing. A PNA moiety can serve as a joining element, while an oligopeptide, protein, or protein fragment provides a small structural element and, in specific embodiments, the structural element further comprises a functional element, as depicted schematically in FIGS. 19(A-B). As shown in FIGS. 19(A-B), two PNA/oligopeptide units can dimerize to form a single assembly unit. The PNA portion provides joining elements A and B′, while the oligopeptide portion forms two coiled coil structural elements.

Like DNA, PNA/PNA molecules bind most stably in an antiparallel fashion (Wittung et al., 1994, DNA-like double helix formed by peptide nucleic acid, Nature 368: 561-63). For PNA molecules the amino terminus is equivalent to the 5′ end of a corresponding DNA sequence (FIG. 1). Leucine zipper dimers normally bind in a parallel fashion (amino terminus adjacent to amino terminus) (Harbury et al., 1993, A switch between two-, three-, and four-stranded coiled coils in GCN4 leucine zipper mutants, Science 262: 1401-07). Therefore, all the molecules depicted in the assembly units shown in FIG. 2 are shown in a parallel orientation (the amino terminals are the 5′ ends to the left and the carboxy terminals are the 3′ ends to the right).

In certain embodiments, the assembly unit can have a randomly coiled peptide that comprises a functional element, F, in the internal or center portion of the dimer (FIG. 2A) or at the end of the PNA molecule opposite the end comprising the joining element. The two functional elements may be the same or different. The joining elements are designed to obviate uncontrolled assembly to allow for staged assembly using such an assembly unit. In this illustration, at least two complementary pairs of PNA sequences are used. There must be no self-complementation or cross-complementation between the joining pairs.

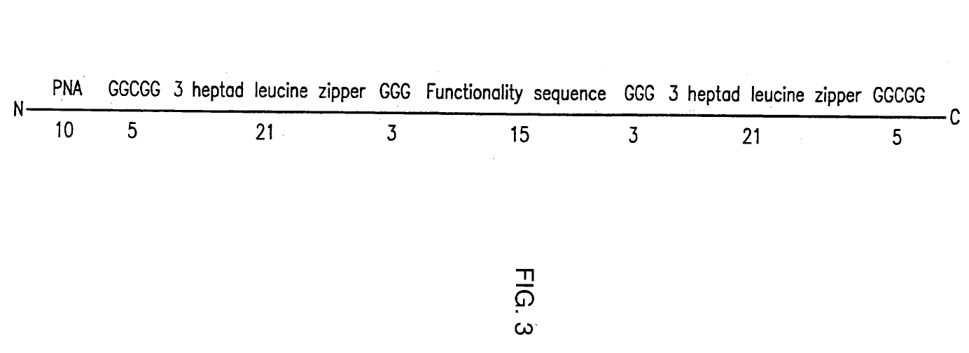

FIG. 3 shows the order of elements of the upper synthetic protein monomer forming the staged assembly subunit shown in FIG. 2A. The order of the elements in the corresponding lower unit would be identical except that the PNA element is at the C-terminus. This reflects the parallel arrangement of the leucine zippers aligning the two units. The functionality sequence encodes the region at which a functional element may be added to the assembly subunit. Glycines separate each element to reduce steric interference between elements. Numbers below the line indicate the typical length in residues of each element.

Formation of a PNA/oligopeptide assembly unit structure may be monitored using the same methodologies commonly known in the art that are used for monitoring protein folding. For example, the oligopeptide portion can be modeled with software that predicts the formation of coiled-coils, e.g. Multicoil (Wolf et al., 1997, MultiCoil: A program for predicting two- and three-stranded coiled coils, Protein Science 6: 1179-89), Paircoil (Berger et al., 1995, Predicting coiled coils by use of pairwise residue correlations, Proc. Natl. Acad. Sci. USA, 92: 8259-63), COILS (Lupas et al., 1991, Predicting coiled coils from protein sequences, Science 252: 1162-64; Lupas, 1996, Prediction and analysis of coiled-coil structures, Meth. Enzymology 266: 513-25) and Macstripe (Lupas et al, 1991, Predicting Coiled Coils from Protein Sequences, Science 252: 1162-64). Standard techniques such as measurement of circular dichroism (CD), e.g., a CD spectrum, can also be used to monitor oligopeptide folding. Moreover, modeling of formation of a joining pair comprising PNA joining elements follows the same rules as DNA-DNA complementary pairing. PNA joining pairs are preferably evaluated using any of a variety of commercial software packages, e.g., Amplify (University of Wisconsin, Madison Wis.), Vector NTI (InforMax, Bethesda Md.), and GCG Wisconsin Package (Accelrys Inc., Burlington Mass.).

PNA/oligopeptide assembly units differ from other types of assembly units (such a pilin-based immunoglobulin-based assembly units) in several aspects. PNA/oligopeptide assembly units are hybrids of two different classes of biological molecules—PNA and oligopeptide—and are, therefore, chemically synthesized rather than biologically synthesized. Accordingly, a strict level of quality control and testing for each batch of such PNA-containing assembly units is required. These tests include, e.g., sandwich ELISAs and tests for circular dichroism for protein/protein interactions, evaluation of temperatures for PNA joining elements, and SDS-PAGE for determining the percent of full-length molecules.

The α-helical oligopeptide portion of an assembly unit is about 1 nm long per heptad repeat in embodiments where, for example, leucine zipper protein domains are used as structural elements in the construction of an assembly unit (Harbury et al., 1994, Crystal structure of an isoleucine-zipper trimer, Nature 371: 80-83). In embodiments in which an assembly unit has four to six heptads (28-42 amino acids), the structural element is about 4-6 nm long. The PNA joining element is structurally similar to DNA and has a length of about 0.34 nm/base. Therefore, in certain embodiments, a joining element of 10-18 residues will be about 3 to 6 nm in length and, therefore, such an assembly unit will be about 7-12 nm long.

PNA/oligopeptide assembly units also differ from other embodiments of the invention disclosed herein in that they are generally less rigid. In a specific embodiment, a PNA-peptide assembly unit has a structural element comprising a leucine zipper structure. Such a PNA-peptide assembly unit has an alpha helical portion that has some flexibility although, in certain embodiments, the presence of two or three helix bundles is not as flexible as an isolated a-helical coil. The PNA portion is relatively flexible, so that a structure assembled according to the staged assembly method of the invention from these units may be more analogous to a string of soft beads than to a rigid rod. In addition, a flexible domain (e.g., a tri-, tetra- or pentaglycine) which, in certain embodiments, links joining elements to structural elements, will add to the flexibility of the assembly unit and higher order structures. Two- and three-dimensional nanostructures made of these units are somewhat flexible as free units. However, upon attachment at multiple points to a solid support or matrix, the nanostructure can be made rigid by applying tension to the overall structure, in a manner analogous to the stiffening of a rope net or a spider web by application of a tensioning force.

The coiled coil structural elements also allow for flexibility in the design and construction of assembly units and the nanostructures fabricated from those assembly units. Generally, simple leucine zipper type coiled coils, as disclosed above, are not stable enough to hold the assembly units together by themselves but are stabilized by disulfide bridges (see above). Four helical bundles that are found, for example, in the Rop protein, are generally stable enough, at normal room temperature and can be lengthened, as needed, to provide the stability that is required for formation of assembly units. In addition, the distance between functional elements can be adjusted by changing the length of the coiled coils and by adding flexible peptide segments between, e.g., joining and functional elements. This would lead, in certain embodiments, to a flexible nanostructure more akin to a beads-on-a-string type of architecture.

Because the PNA/protein assembly molecule shares a common backbone, it can be synthesized as a single molecule. It is unnecessary to join the two components together after they are synthesized separately. Custom, contract PNA/protein synthesis is available commercially from PerSeptive Biosystems (division of Applied Biosystems, Framingham Mass.).

The sequence of each PNA joining element is critical to correct assembly. While designing complementary pairs is relatively easy to those skilled in the art, it is important to ascertain that there is no complementary base pairing between PNAs that will be part of the same assembly unit. There are a variety of DNA software packages known to skilled in the art, that can be used to analyze nucleotide sequences for complementarity, e.g., Amplify (University of Wisconsin, Madison Wis.), Vector NTI (InforMax, Bethesda Md.), and GCG Wisconsin Package (Accelrys Inc., Burlington Mass.). PNA segments that have internal complementarity can form hairpin loops and are preferably avoided according to the staged-assembly methods disclosed herein.

Table 1 below lists exemplary PNA sequences that can be comprised in joining elements in PNA/protein assembly units, and gives examples of usable and unusable sequences. In preferred embodiments, one member of the PNA joining pair is attached to a single assembly unit. The corresponding member of the joining pair is the direct complementary sequence, and is attached to another assembly unit. The sequences in Table 1 are listed in amino to carboxy (5′ to 3′) orientation.

TABLE 1
PNA Sequences for Use as Joining Elements in PNA/Protein
Assembly Units
Compatible binding element pairs (for two assembly units
having the general form of A . . . B′and B . . . A′;
* represents the remainder of the assembly unit).

| Complementary | | | |
|---|---|---|---|
| binding pair 1 | | Complementary binding pair 2 | |
| A | A′ | B | B′ |
| *gggggggggg | cccccccccc* | *aaaaaaaaaa | tttttttttt* |
| (SEQ ID NO:1) | (SEQ ID NO:2) | (SEQ ID NO:3) | (SEQ ID NO:4) |
| *gggggttttt | cccccaaaaa* | *tttttggggg | aaaaaccccc* |
| (SEQ ID NO:5) | (SEQ ID NO:6) | (SEQ ID NO:7) | (SEQ ID NO:8) |
| *acacacacac | tgtgtgtgtg* | *tctctctctc | agagagagag* |
| (SEQ ID NO:9) | (SEQ ID NO:10) | (SEQ ID NO:11) | (SEQ ID NO:12) |
| *atagacagat | tatctgtcta* | *cgctgagatg | gcgactctac* |
| (SEQ ID NO:13) | (SEQ ID NO:14) | (SEQ ID NO:15) | (SEQ ID NO:16) |
| *aacagctaac | ttgtcgattg* | *tttggatatg | aaacctatac* |
| (SEQ ID NO:17) | (SEQ ID NO:18) | (SEQ ID NO:19) | (SEQ ID NO:20) |
| *gttctggtaa | caagaccatt* | *ttttgcgaac | aaaacgctta* |
| (SEQ ID NO:21) | (SEQ ID NO:22) | (SEQ ID NO:23) | (SEQ ID NO:24) |
| *ctcaatttgc | gagttaaacg* | *tggggatgtt | acccctacaa* |
| (SEQ ID NO:25) | (SEQ ID NO:26) | (SEQ ID NO:27) | (SEQ ID NO:28) |
| *cacacaggaa | gtgtgtcctt* | *acagctatga | tgtcgatact* |
| (SEQ ID NO:29) | (SEQ ID NO:30) | (SEQ ID NO:31) | (SEQ ID NO:32) |
| *gagcctccag | ctcggaggtc* | *ttgttgaacc | aacaacttgg* |
| (SEQ ID NO:33) | (SEQ ID NO:34) | (SEQ ID NO:35) | (SEQ ID NO:36) |
| *gggtgcaggt | cccaogtcca* | *tcatttgctt | agtaaacgaa* |
| (SEQ ID NO:37) | (SEQ ID NO:38) | (SEQ ID NO:39) | (SEQ ID NO:40) |
| *ccaagttcac | ggttcaagtg* | *gctttatcca | cgaaataggt* |
| (SEQ ID NO:41) | (SEQ ID NO:42) | (SEQ ID NO:43) | (SEQ ID NO:44) |
| *cgggtacggt | gcccatgcca* | *cagaatgact | gtcttactga* |
| (SEQ ID NO:45) | (SEQ ID NO:46) | (SEQ ID NO:47) | (SEQ ID NO:48) |
| *ccccaagcat | ggggttcgta* | *gtggtttagt | caccaaatca* |
| (SEQ ID NO:49) | (SEQ ID NO:50) | (SEQ ID NO:51) | (SEQ ID NO:52) |

Complementary binding pairs forming triple helices. “OOOO” represents residues with no base, essentially glycines that allow the PNA to fold back on itself to form the triple helix.

| A | A′ |
|---|---|
| *cccccccOOOOccccccc | ggggggg* |
| (SEQ ID NO:53) | (SEQ ID NO:54) |
| *cccttttOOOOttttccc | gggaaaa* |
| (SEQ ID NO:55) | (SEQ ID NO:56) |
| *tctctctOOOOtctctct | agagaga* |
| (SEQ ID NO:57) | (SEQ ID NO:58) |
| *cttcctcOOOOctccttc | gaaggag* |
| (SEQ ID NO:59) | (SEQ ID NO:60) |

Sequences Unsuitable as Binding Elements

Sequences with cross-complementation (complementary sequences underlined)

| A | B′ |
|---|---|
| *ggactatgtt | gatacaagat* |
| (SEQ ID NO:61) | (SEQ ID NO:62) |
| *tctgtattgg | ataacctgac* |
| (SEQ ID NO:63) | (SEQ ID NO:64) |

Sequences forming hairpin loops

*gggttttccc
(SEQ ID NO:65)
*gatcttgatc
(SEQ ID NO:66)

FIGS. 19(A-B) contains line diagrams of two possible embodiments of synthetic molecules that can be used in the construction of an assembly unit useful for the present staged assembly methods. As shown in FIGS. 19(A-B), two PNA/oligopeptide units can dimerize to form a single assembly unit. Two possible assembly units are shown in FIG. 2A and FIG. 2B. The PNA portion provides joining elements A and B′, while the oligopeptide portion forms two coiled coil structural elements (S) stabilized by disulfide bonds at either end. One or more functional units (F), comprised of, e.g., protein segments, may also be incorporated into the assembly unit. In certain embodiments, the assembly unit can have a randomly coiled peptide that comprises a functional element, F, in the internal or center portion of the dimer (FIG. 2A) or at the end of the PNA molecule opposite the end comprising the joining element (FIG. 2B).

In this example, the order of elements (i.e., joining structural, and/or functional elements) in the corresponding next assembly unit (i.e., one to be added next during staged assembly) would be identical, except that the PNA element would be at the C-terminus. This reflects the parallel arrangement of the leucine zippers. Glycines separate each element to reduce steric interference between elements.

PNA Functional Elements

In another embodiment, functional elements (depicted as “F”) comprising peptide sequences are placed in two possible locations in an assembly unit formed by leucine zipper dimerization. Sequences can be added to the opposite end of the peptide from, e.g., a PNA, or can be inserted between two shorter α-helices, as shown in FIG. 2.

Table 2 sets forth several non-limiting, illustrative examples of functional elements.

TABLE 2
Peptides That Can Be Used as Functional Elements in Peptide/PNA
Units

| Amino acid sequence | | Origin/activity/reference |
|---|---|---|
| Epitopes | | |
| SGFNADYEASSSRC | (SEQ ID NO:67) | human fos |
| PIDMESQERIKAERKRM | (SEQ ID NO:68) | v-jun |
| EQKLISEEDL | (SEQ ID NO:69) | c-myc |
| EEYSAMRDQYMRTGE | (SEQ ID NO:70) | v-H-ras |
| QPELAPEDPED | (SEQ ID NO:71) | herpes simplex virus |
| MASMTGGQQMG | (SEQ ID NO:72) | bacteriophage T7 gene 10 |
| YGGFL | (SEQ ID NO:73) | β-endorphin |
| Biotin analogues | | |
| (bind to strep- | | |
| tavidin) | | |
| ISFENTWLWHPQFSS | (SEQ ID NO:74) | |
| | | Devlin et al., 1990, Random peptide libraries: A |
| | | source of specific protein binding molecules, |
| | | Science 249: 404-406 |
| TPHPQ | (SEQ ID NO:75) | |
| | | Lam et al., 1991, A new type of synthetic peptide |
| | | library for identifying ligand-binding activity, |
| | | Nature 354: 82-84 |
| MHPMA | (SEQ ID NO:76) | |
| | | Lam et al., 1991, A new type of synthetic peptide |
| | | library for identifying ligand-binding activity, |
| | | Nature 354: 82-84 |
| His tags (bind to | | |
| nickel and nickel | | |
| conjugates) | | |
| H₆₋₁₀ | | |
| Peptides (bind to | | |
| specific protein | | |
| targets) | | |
| KETAAAKFERQHMDS | (SEQ ID NO:77) | |
| | | binds S-protein conjugate |
| | | Richards and Wyckoff, in “The Enzymes”Vol. IV, |
| | | P. D. Boyer ed., Academic Press, New York, pp. |
| | | 647-806 |
| RRASV | (SEQ ID NO:78) | |
| | | protein kinase A phosphorylation target |
| | | de Arruda and Burgess, 1996, pET-33B(+): A pET |
| | | vector that contains a protein kinase A recognition |
| | | sequence, Novagen Innovations 4a: 7-8 |
| Peptides (bind to | | |
| GaAs) | | |
| VTSPDSTTGAMA | (SEQ ID NO:79) | Whaley et al., 2000, Selection of peptides |
| AASPTQSMSQAP | (SEQ ID NO:80) | with semiconductor binding specificity for |
| AQNPSDNNTHTH | (SEQ ID NO:81) | directed nanocrystal assembly, Nature 405: |
| ASSSRSHFGQTD | (SEQ ID NO:82) | 665-668 |
| WAHAPQLASSST | (SEQ ID NO:83) | |
| ARYDLSIPSSES | (SEQ ID NO:84) | |
| TPPRPIQYNHTS | (SEQ ID NO:85) | |
| SSLQLPENSFPH | (SEQ ID NO:86) | |
| GTLANQQIFLSS | (SEQ ID NO:87) | |
| HGNPLPMTPFPG | (SEQ ID NO:88) | |
| RLELAIPLQGSG | (SEQ ID NO:89) | |

In one embodiment, the functional element comprises a PNA segment. Just as PNA can be placed at the end of the monomer during synthesis to serve as a joining element, a segment of PNA, comprising residues capable of base-paring, can be placed into the middle of a synthesized peptide subunit to serve as a functional element. This permits the fabrication of a precisely branched nanostructure, or a nanostructure comprising a PNA-conjugated joining element that is precisely attached to the nanostructure by base-pairing interactions with the structural element-embedded PNA functional element. In preferred embodiments, functional elements, and/or bridging cysteine residues, are generally separated from neighboring structural and/or joining elements by a peptide segment of about two to five glycine residues, so that the protein/peptide domains can form independently.

Other Elements

In certain embodiments of the present invention, an assembly unit comprises a structural element. As noted above, that structural element may be a leucine zipper. More generally, the structural element generally has a rigid structure (although in certain embodiments, described below, the structural element may be non-rigid). The structural element is preferably a defined peptide, protein or protein fragment of known size and structure that comprises at least about 50 amino acids and, generally, fewer than 2000 amino acids. Peptides, proteins and protein fragments are preferred since naturally-occurring peptides, proteins and protein fragments have well-defined structures, with structured cores that provide stable spatial relationships between and among the different faces of the protein. This property allows the structural element to maintain pre-designed geometric relationships between the joining elements and functional elements of the assembly unit, and the relative positions and stoichiometries of assembly units to which it is bound.

The use of proteins as structural elements has particular advantages over other choices such as inorganic nanoparticles. Most populations of inorganic nanoparticles are heterogeneous, making them unattractive scaffolds for the assembly of a nanostructure. In most populations, each inorganic nanoparticle is made up of a different number of atoms, with different geometric relationships between facets and crystal faces, as well as defects and impurities. A comparably sized population of proteins is, by contrast, very homogeneous, with each protein comprised of the same number of amino acids, each arranged in approximately the same way, differing in arrangement, for the most part, only through the effect of thermal fluctuations. Consequently, two proteins designed to interact with one another will always interact with the same geometry, resulting in the formation of a complex of predictable geometry and stoichiometry. This property is essential for massively parallel “bottom-up” assembly of nanostructures.

A structural element may be used to maintain the geometric relationships among the joining elements and functional elements of a nanostructure. As such, a rigid structural element is generally preferred for construction of nanostructures using the staged assembly methods described herein. This rigidity is typical of many proteins and may be conferred upon the protein through the properties of the secondary structural elements making up the protein, such as α-helices and β-sheets.

Structural elements may be based on the structure of proteins, protein fragments or peptides whose three-dimensional structure is known or may be designed ab initio. Examples of proteins or protein fragments that may be utilized as structural elements in an assembly unit include, but are not limited to, antibody domains, diabodies, single-chain antibody variable domains, and bacterial pilins.

In some embodiments, structural elements, joining elements and functional elements may be of well-defined extent, separated, for example, by glycine linkers. In other embodiments, joining elements may involve peptides or protein segments that are integral parts of a structural element, or may comprise multiple loops at one end of a structural element, such as in the case of the complementarity determining regions (CDRs) of antibody variable domains (Kabat et al., 1983, Sequences of Proteins of Immunological Interest, U.S. Department of Health and Human Services). A CDR is a joining element that is an integral part of the variable domain of an antibody. The variable domain represents a structural element and the boundary between the structural element and the CDR making up the joining element (although well-defined in the literature on the basis of the comparisons of many antibody sequences) may not always be completely unambiguous structurally. There may not always be a well-defined boundary between a structural element and a joining element, and the boundary between these domains, although well-defined on the basis of their respective utilities, may be ambiguous spatially.

Structural elements of the present invention comprise, e.g., core structural elements of naturally-occurring proteins that are then modified to incorporate joining elements, functional elements, and/or a flexible domain (e.g., a tri-, tetra- or pentaglycine), thereby providing useful assembly units. Consequently, in certain embodiments, structures of existing proteins are analyzed to identify those portions of the protein or part thereof that can be modified without substantially affecting the rigid structure of that protein or protein part.

For example, in certain embodiments, the amino acid sequence of surface loop regions of a protein or structural element are altered with little impact on the overall folding of the protein. The amino acid sequences of a surface loop of a protein are generally preferred as amino acid positions into which the additional amino acid sequence of a joining element, a functional element, and/or a flexible domain may be inserted, with the lowest probability of disrupting the protein structure. Determining the position of surface loops in a protein is carried out by examination of the three-dimensional structure of the protein or a homolog thereof, if three-dimensional atomic coordinates are available, using, for example, a public-domain protein visualization computer program such as RASMOL (Sayle et al., 1995, RasMol: Biomolecular graphics for all, Trends Biochem. Sci. (TIBS) 20(9): 374-376; Saqi et al., 1994, PdbMotif—a tool for the automatic identification and display of motifs in protein structures, Comput. Appl. Biosci. 10(5): 545-46). In this manner, amino acids included in surface loops, and the relative spatial locations of these surface loops, can be determined.

If the three-dimensional structure of the protein being engineered is not known, but that of a close homolog is known (as is the case, for example, for essentially all antibody molecules), the amino acid sequence of the molecule of interest, or a portion thereof, can be aligned with that of the molecule whose three-dimensional structure is known. This comparison (done, for example, using BLAST (Altschul et al., 1997, Gapped BLAST and PSI-BLAST: a new generation of protein database search programs, Nucleic Acids Res. 25: 3389-3402) or LALIGN (Huang and Miller, 1991, A time efficient, linear-space local similarity algorithm, Adv. Appl. Math. 12: 337-357) allows identification of all the amino acids in the protein of interest that correspond to amino acids that constitute surface loops (β-turns) in the protein of known three-dimensional structure. In regions in which there is high sequence similarity between the two proteins, this identification is carried out with a high level of certainty. Once a putative loop is identified and altered according to methods disclosed herein, the resultant construct is tested to determine if it has the expected properties. This analysis is performed even in those instances where identification of the loop is highly reliable, e.g. where that determination is based upon a known three-dimensional protein structure.

Structural elements comprising leucine zipper-type coiled coils can also be employed in assembly units in the nanostructures of the invention. In certain embodiments, the invention encompasses structural elements comprising leucine zipper-type coiled coils for use in the construction of nanostructures using the staged assembly methods of the invention. Leucine zippers are well-known, α-helical protein structures (Oas et al., 1994, Springs and hinges: dynamic coiled coils and discontinuities, TIBS 19: 51-54; Branden et al., 1999, Introduction to Protein Structure 2nd ed., Garland Publishing, Inc., New York) that are involved in the oligomerization of proteins or protein monomers into dimeric, trimeric, and tetrameric structures, depending on the exact sequence of the leucine zipper domain (Harbury et al., 1993, A switch between two-, three-, and four-stranded coiled coils in GCN4 leucine zipper mutants, Science 262: 1401-07). While only dimers are disclosed herein for simplicity, it would be apparent to one of ordinary skill in the art that trimeric and tetrameric units may also be used for the construction of assembly units for use in staged assembly of nanostructures according to the methods disclosed herein. In certain embodiments, trimeric and tetrameric units could be especially useful for incorporation of functional elements that, e.g., require two or more chemical moieties for proper activity, for example, the incorporation of two cysteine moieties for binding of gold particles. Several non-limiting examples of leucine-zipper domains are provided in Table 3 below.

Table 3 shows canonical leucine zippers and high stability dimerization sequences. The top line shows register of the repeat unit. Residues in the a and d positions are generally hydrophobic and control the oligomerization. Residues in the e and g positions are generally charged and create salt bridges to stabilize the oligomerization.

TABLE 3
Canonical Leucine Zippers and High Stability
Dimerization Sequences

| | |
|---|---|
| abcdefgabcdefgabcdefgabcdefgabcdefg | |
| GCN4 | |
| MKQLEDKVEELLSKNYHLENEVARLKKL | (SEQ ID NO:90) |
| c-Fos | |
| TDTLQAETDQLEDEKYALQTEIANLLKE | (SEQ ID NO:91) |
| c-Jun | |
| AARLEEKVKTLKAQNYELASTANMLREQ | (SEQ ID NO:92) |
| C/EBPb | |
| VLETQHKNERLTAEVEQLQKKLSTLSREFKQLRNL | (SEQ ID NO:93) |
| ATF4 | |
| CKELTGENEALEKKADSLKERTQYLAKETEEVKDL | (SEQ ID NO:94) |
| c-myc | |
| CGGVQAEEQKLISEEDLLRKRREQLKHKLEQLX | (SEQ ID NO:95) |
| Max | |
| CGGMRRKNDTHQQDIDDLKRQNALLEQQVPALX | (SEQ ID NO:96) |
| CREB | |
| VKSLENRVAVLENQNKTLIEELKALKDLYSHK | (SEQ ID NO:97) |
| PAP1 | |
| VVTLKELHSSTTLENDQLRQKVRQLEEELRTLK | (SEQ ID NO:98) |

Many naturally occurring leucine zippers may be used according to the methods of the invention, including those found in the yeast transcription factor GCN4 and in the mammalian Fos, Jun and Myc oncogenes. Additional proteins containing leucine zippers and other coiled coil-type oligomerization sequences can be identified by searching public protein databases such as SWISS-PROT/TrEMBL (Bairoch and Apweiler, 2000, The SWISS-PROT protein sequence database and its supplement TrEMBL in 2000, Nucl. Acids Res. 28: 45-48). Table 4 shows the results of such a search, using the keywords “coiled coil” and “dimer.”

In Table 4, the common names of genes are listed, as well as their SWISS-PROT accession numbers, sequence description and sequence. The SWISS-PROT accession number is a unique identifier for a sequence record. An accession number applies to the complete record and is usually a combination of a letter(s) and numbers, such as a single letter followed by five digits (e.g., Q12345) or a combination of six letters and digits (e.g., Q1Z2F3). The coiled coil sequences are underlined.

TABLE 4
Examples of Proteins Containing Coiled Coil Dimerization Sequences That
Can Be Used for Structural Elements of Assembly Units

| | | | |
|---|---|---|---|
| Sequence | Accession | Sequence | |
| ID | number | description | Sequence |

| | | | | | | |
|---|---|---|---|---|---|---|
| SWISS_PR | O54931 | A-kinase anchor | MEIGVSVAECKSVPGVTSTPHSKDHSSPFYSPS | (SEQ ID NO: 99) | | |
| OT: | O54932 | protein 2 | HNGLLADHHESLDNDVAREIQYLDEVLEANCCD | | | |
| AKA2_MOU | O54933 | (Protein kinase | SSVDGTYNGISSPEPGAAILVSSLGSPAHSVTE | | | |
| SE | | A anchoring | AEPTEKASGRQVPPHIELSRIPSDRMAEGERAN | | | |
| | | protein | GHSTDQPQDLLGNSLQAPASPSSSTSSHCSSRD | | | |
| | | 2)\|(PRKA2) | GEFTLTTLKKEAKFELRAFHEDKKPSKLFEEDE | | | |
| | | (AKAP expressed | REKEQFCVRKVRPSEEMIELEKERRELIRSQAV | | | |
| | | in kidney and | KKNPGIAAKWWNPPQEKTIEEQLDEEHLESHRR | | | |
| | | lung) (AKAP-KL) | YKERKEKRAQQEQLQLQQQQQQQLQQQQLQQQQ | | | |
| | | | LQQQQLQQQLQQQQLSTSQPCTAPAAHKHLDGI | | | |
| | | | EHTKEDVVTEQIDFSAARKQFQLMENSRQTLAK | | | |
| | | | GQSTPRLFSIKPYYKPLGSIHSDKPPTILRPAT | | | |
| | | | VGGTLEDGGTQAAKEQKAPCVSESQSAGAGPAN | | | |
| | | | AATQGKEGPYSEPSKRGPLSKLWAEDGEFTSAR | | | |
| | | | AVLTVVKDEDHGILDQFSRSVNVSLTQEELDSG | | | |
| | | | LDELSVRSQDTTVLETLSNDFSMDNISDSGASN | | | |
| | | | ETTSALQENSLADFSLPQTPQTDNPSEGREGVS | | | |
| | | | KSFSDHGFYSPSSTLGDSPSVDDPLEYQAGLLV | | | |
| | | | QNAIQQAIAEQVDKAEAHTSKEGSEQQEPEATV | | | |
| | | | EEAGSQTPGSEKPQGMFAPPQVSSPVQEKRDIL | | | |
| | | | PKNLPAEDRALREKGPSQPPTAAQPSGPVNMEE | | | |
| | | | TRPEGGYFSKYSEAAELRSTASLLATQESDVMV | | | |
| | | | GPFKLRSRKGRTLSMIEEEIPAAQEREEELKRQ | | | |
| | | | RGVRQSTPSPRAKNAPSLPSRTTCYKTAPGKIE | | | |
| | | | KVKPPPSPTTEGPSLQPDLAPEEAAGTQRPKNL | | | |
| | | | MQTLMEDYETHKSKRRERMDDSSYTSKLLSCKV | | | |
| | | | TSEVLEATRVNRRKSASGLALGGRDLR | | | |
| SWISS_PR | Q99996 | A-kinase anchor | MEDEERQKKLEAGKAKIEELSLAFLVRQLAQFR | (SEQ ID NO:10) | | |
| OT: | Q9UQQ4 | protein 9 | QRKAQSDGQSPSKKQKKKRKTSSSKHDVSAHHD | | | |
| AKA9_HUM | Q9UQH3 | (Protein kinase | LNIDQSQCHEMYINSSQRVESTVIPESTIMRTL | | | |
| AN | Q9Y6Y2 | A anchoring | HSGEITSHEQGFSVELESEISTTANDCSSEVNG | | | |
| | 014869 | protein | CSFVMRTGKPTNTLLREEEFGVDDSYSEQGAQDS | | | |
| | 043355 | 9)\|(PRKA9) (A-kinase | PTHLEMMESELAGKQHEIEELNRELEEMRVTYG | | | |
| | 094895 | anchor | TEGLQQLQEFEAAIKQRDGIITQLTANLQQARR | | | |
| | Q9YGB8 | protein 450 | EKDETMREFLELTEQSQKLQIQFQQLQASETLR | | | |
| | | kDa) (AKAP 450) | NSTHSSTAADLLQAKQQILTHQQQLEEQDHLLE | | | |
| | | (A-kinase | DYQKKKEDFTMQTSFLQEKIKVYEMEQDKKVEH | | | |
| | | anchor\|protein | SNKEEIQEKETIIEELNTKIIEEEKKTLELKDK | | | |
| | | 350 kDa) (AKAP | LTTADKLLGELQEQIVQKLINQETKNMKLELTNSK | | | |
| | | 350) (hgAKAP | QKERQSSEEIKQLMGTVEELQKRNHKDSQFETD | | | |
| | | 350) (AKAP 120 | IVQRMEQETQRKLEQLRAELDEMYGQQIVQMKQ | | | |
| | | like | ELIRQHMAQMEEMKTRHKGEMENALRSYSNITV | | | |
| | | protein)\|(Hyper | NEDQIKLMNVAINELHIKLQDTNSQKEKLKEEL | | | |
| | | ion protein) | GLILEEKCALQRQLEDLVEELSFSREQIQRANQ | | | |
| | | (Yotiao | TIAEQESKLNEAHKSLSTVEDLKAEIVSASESR | | | |
| | | protein) | KELELKHEAEVTNYKIKLEMLEKEKNAVLDRMA | | | |
| | | (Centrosome- and | ESQEAELERLRTQLLFSHEEELSKLKEDLEIEH | | | |
| | | golgi-localized\| | RINILEKLKDNLGIHYKQQIDGLQNEMSQKIETM | | | |
| | | PKN-associated | QFEKDNLITKQNQLILEISKLKDLQQSLVNSKS | | | |
| | | | EEMTLQINELQKEIETLRQEEKEKGTLEQEVQE | | | |
| | | protein) | LQLKTELLEKQMKEKENDLQEKFAQLEAENSIL | | | |
| | | NAP) | KDEKKTLEDMLKIHTPVSQEERLIFLDSIKSKS | | | |
| | | | KDSVWEKEIEILIEENEDLKQQCIQLHEEIEKQ | | | |
| | | | RNTFSFAEKNFEVNYQELQEEYACLLKVKDDLE | | | |
| | | | DSKNKQELEYKSKLKALNEELHLQRINPTTVKM | | | |
| | | | KSSVFDEDKTFVAETLEMGEVVTEKDTTELMEKL | | | |
| | | | EVTKREKLELSQRLSDLSEQLKQKHGEISFLNE | | | |
| | | | EVKSLKQEKEQVSLRCRELEIIINHNRAENVQS | | | |
| | | | CDTQVSSLLDGVVTMTSRGAEGSVSKVNKSEGE | | | |
| | | | ESKIMVEDKVSFENMTVGEESKQEQLILDHLPS | | | |
| | | | VTKESSLRATQPSENDKLQKELNVLKSEQNDLR | | | |
| | | | LQMEAQRICLSLVYSTHVDQVREYMENEKDKAL | | | |
| | | | CSLKEELIFAQEEKIKELQKIHQLELQTMKTQE | | | |
| | | | TGDEGKPLHLLIGKLQKAVSEECSYFLQTLCSV | | | |
| | | | LGEYYTPALKCEVNAEDKENSGDYISENEDPEL | | | |
| | | | QDYRYEVQDFQENMHTLLNKVTEEYNKLLVLQT | | | |
| | | | RTSKIWGQQTDGMKLEFGEENLPKEETEFLSIH | | | |
| | | | SQMTNLEDIDVNHKSKLSSLQDLEKTKLEEQVQ | | | |
| | | | ELESLISSLQQQLKETEQNYEAEIHCLQKRLQA | | | |
| | | | VSESTVPPSLPVDSVVITESDAQRTMYPGSCVK | | | |
| | | | KNIDGTIEFSGEFGVKEETNIVKLLEKQYQEQL | | | |
| | | | EEEVAKVIVSMSIAFAQQTELSRISGGKENTAS | | | |
| | | | SKQAHAVCQQEQHYFNEMKLSQDQIGFQTFETV | | | |
| | | | DVKFKEEFKPLSKELGEHGKEILLSNSDPHDIP | | | |
| | | | ESKDCVLTISEEMFSKDKTFIVRQSIHDEISVS | | | |
| | | | SMDASRQLMLNEEQLEDMRQELVRQYQEHQQAT | | | |
| | | | ELLRQAHMRQMERQREDQEQLQEEIKRLNRQLA | | | |
| | | | QRSSIDNENLVSERERVLLEELEALIKQLSLAGR | | | |
| | | | EKLCCELRNSSTQTQNGNENQGEVEEQTFKEKE | | | |
| | | | LDRKPEDVPPETLSNERYALQKANNRLLKILLE | | | |
| | | | VVKTTAAVEETIGRHVLGILDRSSKSQSSASLI | | | |
| | | | WRSEAEASVKSCVHEEHTRVTDESIPSYSGSDM | | | |
| | | | PRNDINMWSKVTEEGTELSQRLVRSGFAGTEID | | | |
| | | | PENEELMLNISSRLQAAVEKLLEAISETSSQLE | | | |
| | | | HAKVTQTELMRESFRQKQEATESLKCQEELRER | | | |
| | | | LHEESRAREQLAVELSKAEGVIDGYADEKTLFE | | | |
| | | | RQIQEKTDIIDRLEQELLCASNRLQELEAEQQQ | | | |
| | | | IQEERELLSRQKEAMKAEAGPVEQQLLQETEKL | | | |
| | | | MKEKLEVQCQAEKVRDDLQKQVKALEIDVEEQV | | | |
| | | | SRFIELEQEKNTELMDLRQQNQALEKQLEKMRK | | | |
| | | | FLDEQAIDREHERDVFQQEIQKLEQQLKVVPRF | | | |
| | | | QPISEHQTREVEQLANHLKEKTDKCSELLLSKE | | | |
| | | | QLQRDIQERNEEIEKLEFRVRELEQALLVSADT | | | |
| | | | FQKVEDRKHFGAVEAKPELSLEVQLQAERDAID | | | |
| | | | RKEKEITNLEEQLEQFREELENKNEEVQQLHMQ | | | |
| | | | LEIQKKESTTRLQELEQENKLFKDDMEKLGLAI | | | |
| | | | KESDAMSTQDQHVLFGKFAQIIQEKEVEIDQLN | | | |
| | | | EQVTKLQQQLKITTDNKVIEEKNELIRDLETQI | | | |
| | | | ECLMSDQECVKRNREEEIEQLNEVIEKLQQELA | | | |
| | | | NIGQKTSMNAHSLSEEADSLKHQLDVVIAEKLA | | | |
| | | | LEQQVETANEEMTFMKNVLKETNFKMNQLTQEL | | | |
| | | | FSLKRERESVEKIQSIPENSVNVAIDHLSKDKP | | | |
| | | | ELEVVLTEDALKSLENQTYFKSFEENGKGSIIN | | | |
| | | | LETRLLQLESTVSAKDLELTQCYKQIKDMQEQG | | | |
| | | | QFETEMLQKKIVNLQKIVEEKVAAALVSQIQLE | | | |
| | | | AVQEYAKFCQDNQTISSEPERTNIQNLNQLRED | | | |
| | | | ELGSDISALTLRISELESQVVEMHTSLILEKEQ | | | |
| | | | VEIAEKNVLEKEKKLLELQKLLEGNEKKQREKE | | | |
| | | | KKRSPQDVEVLKTTTELFHSNEESGFFNELEAL | | | |
| | | | RAESVATKAELASYKEKAEKLQEELLVKETNMT | | | |
| | | | SLQKDLSQVRDHLAEAKEKLSILEKEDETEVQE | | | |
| | | | SKKACMFEPLPIKLSKSIASQTDGTLKISSSNQ | | | |
| | | | TPQILVKNAQIQINLQSECSSEEVTEIISQFTE | | | |
| | | | KIEKMQELHAAEILDMESRHISETETLKREHYV | | | |
| | | | AVQLLKEECGTLKAVIQCLRSKEVFGFYNMCFS | | | |
| | | | TLCDSGSDWGQGIYLTHSQGFDTASEGEQEESE | | | |
| | | | SATDSFPKKIKGLLRAVHNEGMQVLSLTESPYS | | | |
| | | | DQEDESIQQVSEPWLEERKAYINTISSLKDLIT | | | |
| | | | KMQLQREAEVYDSSQSHESFSDWRGELLLALQQ | | | |
| | | | VFLEERSVLLAAFRTELTALGTTDAVGLLNCLE | | | |
| | | | QRIQEQGVEYQAANECLQKADRRSLLSEIQALH | | | |
| | | | AQMNGRKITLKREQESEKPSQELLEYNIQQKQS | | | |
| | | | QMLEMQVELSSMKDRATELQEQLSSEKMVVAEL | | | |
| | | | KSELAQTKLELETTLKAQHKHLKELEAFRLEVK | | | |
| | | | DKTDEVHLLNDTLASEQKKSRELQWALEKEKAK | | | |
| | | | LGRSEERDKEELEDLKFSLESQKQRNLQLNLLL | | | |
| | | | EQQKQLLNESQQKTESQRMLYDAQLSEEQGRNL | | | |
| | | | ELQVLLESEKVRIREMSSTLDRERELHAQLQSS | | | |
| | | | DGTGQSRPPLPSEDLLKELQKQLEEKHSRIVEL | | | |
| | | | LNETEKYKLDSLQTRQQMEKDRQVHRKTLQTEQ | | | |
| | | | EANTEGQKKMHELQSKVEDLQRQLEEKRQQVYK | | | |
| | | | LDLEGQRLQGIMQEFQKQELEREEKRESRRILY | | | |
| | | | QNLNEPTTWSLTSDRTRNWVLQQKIEGETKESN | | | |
| | | | YAKLIEMNGGGTGCNHELEMIRQKLQCVASKLQ | | | |
| | | | VLPQKASERLQFETADDEDFIWVQENTDEIILQ | | | |
| | | | LQKLTGQQGEEPSLVSPSTSCGSLTERLLRQNA | | | |
| | | | ELTQHISQLTEEKNDLRNMVMKLEEQIRWYRQT | | | |
| | | | GAGRDNSSRFSLHGGANIEAIIASEKEVWNREK | | | |
| | | | LTLQKSLKRAEAEVYKLKAELRNDSLLQTLSPD | | | |
| | | | SEHVTLKRIYGKYLRAESFRKALIYQKKYLLLL | | | |
| | | | LGGFQECEDATLALLARMGGQPAFTDLEVITNR | | | |
| | | | PKGFTRFRSAVRVSIATSRMKFLVRRWHRVTGS | | | |
| | | | VSININRDGFGLNQGAEKTDSFYHSSGGLELYG | | | |
| | | | EPRHTTYRSRSDLDYIRSPLPFQNRYPGTPADF | | | |
| | | | NPGSLACSQLQNYDPDPALTDYITRLEALQRRL | | | |
| | | | GTTQSGSTTQFHAGMRR | | | |
| SWISS_PR | Q28628 | A-kinase anchor | REKLEVQCQAEKVRDDLQKQVKALEIDVEEQVC | (SEQ ID NO:101) | | |
| OT: | | protein 9 | RFIELEQEKNAELMDLRQQNQALEKQLEKMRKM | | | |
| AKA9_PM | (Protein kinase | DLRQQNQALEKQLEKMRKFLDEQAIDREHERDV | | | | |
| IT | | A anchoring | FQQEIQKLEQQLKLVPRFQPISEHQTREVEQLT | | | |
| | | protein | NHLKEKTDKCSETLLLSKEQLQRDVQERNEEIEK | | | |
| | | 9)\|(PRKA9) (A-kinase | LECRVRELEQALLSVQTLSKRWRTRNSFGAVEP | | | |
| | | anchor | KAELCLEVQLQAERDAIDRKEKEITNLEEQLEQ | | | |
| | | protein 120 | FREELENKNEEVQQLHMQLEIQKKESTTRLQEL | | | |
| | | kDa) (AKAP 120) | EQENKLFKDEMEKLGFAIKESDAVSPQDQQVLF | | | |
| | | (Fragment) | QKEAQIIHEKEVEIDRLNEQIIKLQQLKITTD | | | |
| | | | NKVIEEKNELIRDLEAQIECLMSDQERVRKNRE | | | |
| | | | EBIEQLNEVIEKLQQELANIDQKTSVDPSSLSE | | | |
| | | | EADSLKHQLDKVIAEKLALEHQVETTNEEMAVT | | | |
| | | | KNVLKETNFKMNQLTQELCSLKREREKMERIQS | | | |
| | | | VPEKSVNMSVGDLSKDKPEMDLIPTEDALAQLE | | | |
| | | | TQTQLRSSEESSKVSLSSLETKLLQLESTVSTK | | | |
| | | | DLELTQCYKQIQDMREQQRSETEMLQTKIVSLQ | | | |
| | | | KVLEEKVAAALVSQVQLEAVQEYVKLCADKPAV | | | |
| | | | SSDPARTEVPGLSQLAGNTMESDVSALTWRISE | | | |
| | | | LESQLVEMHSSLISEKEQVEIAEKNALEKEKKL | | | |
| | | | QELQKLVQDSETKQRERERQSRLHCDLQVLEST | | | |
| | | | TSEESGVFGELEALRAESAAPKGELANYKELAE | | | |
| | | | KLQEELLVKETNMASLPKELSHVRDQLTEAEDK | | | |
| | | | LSHFSEKEDKTEVQEHGTICILEPCPGQIGESF | | | |
| | | | ASQTEQAVQVNSHTQTPQIPVRSVGIQTHSQSD | | | |
| | | | SSPEEVAEIISRFTEKIEQMRELHAAEILDMES | | | |
| | | | RHISETETLKREHCIAVQLLTEECASLKSLIQG | | | |
| | | | LRMPEGSSVPELTHSNAYQTREVQSSDSGSDWG | | | |
| | | | QGIYLTQSQGFDTASEARGEEGETSTDSFPKKI | | | |
| | | | KGLLRAVHNEGMQVLSLTEGPCGDCEDYPCHQL | | | |
| | | | SESWLEERRAYLSTISSLKDFITKMQVQREVEV | | | |
| | | | YDSSQSHENISDWRGELLLALQQVFLRERSVLL | | | |
| | | | AAFKTELTALGTRDAAGLLNCLEQRIPRTEY | | | |
| SWISS_PR | Q94981 | Ariadne-1 | MDSDNDNDFCDNVDSGNVSSGDDGDDDFGMEVD | (SEQ ID NO:102) | | |
| OT: | | protein (Ari-1) | LPSSADRQMDQDDYQYKVLTTDEIVQHQREIID | | | |
| ARI1_DRO | | | EANLLLKLPTPTTRILLNHFKWDKEKLLEKYFD | | | |
| ME | | | DNTDEFFKCAHVINPFNATEAIKQKTSRSQCEE | | | |
| | | | CEICFSQLPPDSMAGLECGHRFCMPCWHEYLST | | | |
| | | | KIVAEGLGQTISCAAHGCDILVDDVTVANLVTD | | | |
| | | | ARVRVKYQQLITNSFVECNQLLRWCPSVDCTYA | | | |
| | | | VKVPYAEPRRVHCKCQHVFCFACGENWHDPVKC | | | |
| | | | RWLKKWIKKCDDDSETSNWIAANTKECPRCSVT | | | |
| | | | IEKDGGCNHMVCKNQNCKNEFCWVCLQSWEPHG | | | |
| | | | SSWYNCNRYDEDEAKTARDAQEKLRSSLARYLH | | | |
| | | | YYNRYMNHMQSMKFENKLYASVKQKMEEMQQHN | | | |
| | | | MSWIEVQFLKKAVDILCQCRQTLMYTYVFAYYL | | | |
| | | | KKNNQSMIFEDNQKDLESATEMLSEYLERDITS | | | |
| | | | ENLADIKQKVQDKYRYCEKRCSVLLKHVHECYD | | | |
| | | | KEWWEYTE | | | |
| SWISS_PRQ9UBS5 | Gamma-aminobutyric | MLLLLLLAPLFLRPPGAGGAQTPNATSEGCQIT | (SEQ ID NO:103) | | | |
| OT: | O95375 | | HPPWEGGTRYRGLTRDQVKAINFLPVDYEIEYV | | | |
| GBR1_HUM | Q9UQQ0 | acid type B | CRGEREVVGPKVRKCLANGSWTDMDTPSRCVRI | | | |
| AN | O96022 | receptor, | CSKSYLTLENGKVFLTGGDLPALDGARVDFRCD | | | |
| | O95975 | subunit 1 | PDFHLVGSSRSICSQGQWSTPKPHCQVNRTPHS | | | |
| | O95468 | precursor | ERRAVYIGALFPMSGGWPGGQACQPAVEMALED | | | |
| | | (GABA-B\| | VNSRRDILPDYELKLIHHDSKCDPGQATKYLYE | | | |
| | | receptor 1) | LLYNDPIKIILMPGCSSVSTLVAEAARMWNLIV | | | |
| | | (GABA-B-R1) | LSYGSSSPALSNRQRFPTFFRTHPSATLHNPTR | | | |
| | | (Gb1) | VKLFEKWGWKKIATIQQTTEVFTSTLDDLEERV | | | |
| | | | KEAGIEITFRQSFFSDPAVPVKNLKRQDARIIV | | | |
| | | | GLFYETEARKVFCEVYKERLFGKKYVWFLTQWY | | | |
| | | | ADNWFKIYDPSINCTVDEMTEAVEGHITTEIVM | | | |
| | | | LNPANTRSISNMTSQEFVEKLTKRLKRHPEETG | | | |
| | | | GFQEAPLAYDAIWALALALNKTSGGCGRSQVRL | | | |
| | | | EDFNYNNQTITDQIYRANNSSSFEGVSGHVVFD | | | |
| | | | ASGSRMAWTLIEQLQGGSYKKIGYYDSTKDDLS | | | |
| | | | WSKTDKWIQGSPPADQTLVIKTFRFLSQKLFIS | | | |
| | | | VSVLSSLGIVLAVVCLSFNIYNSHVRYIQNSQP | | | |
| | | | NLNNLTAVGCSLALAAVFPLGLDGYHIGRNQFP | | | |
| | | | FVCQARLWLLGLGFSLGYGSMFTKIWWVHTVFT | | | |
| | | | KKEEKKEWRKTLEPWKLYATVGLLVGMDVLTLA | | | |
| | | | IWQIVDPLHRTIETFAKEEPKEDIDVSILPQLE | | | |
| | | | HCSSRKMNTWLGIFYGYKGLLLLLGTFLAYETK | | | |
| | | | SVSTEKINDHRAVGMAIYNVAVLCLITAPVTMI | | | |
| | | | LSSQQDAAFAFASLAIVFSSYITLVVLFVPKMR | | | |
| | | | RLITRGEWQSEAQDTMKTGSSTNNNEEEKSRLL | | | |
| | | | EKENRELEKIIAEKEERVSELRHQLQSRQQLRS | | | |
| | | | RRHPPTPPEPSGGLPRGPPEPPDRLSCDGSRVH | | | |
| | | | LLYK | | | |
| SWISS_PR | P03069 | General control | MSEYQPSLFALNPMGFSPLDGSKSTNENVSAST | (SEQ ID NO:104) | | |
| OT: | P03068 | protein GCN4 | STAKPMVGQLTFDKFIKTEEDPIIKQDTPSNLD | | | |
| GCN4_YEA | | (Amino acid | FDFALPQTATAPDAKTVLPIPELDDAVVESPFS | | | |
| ST | | biosynthesis | SSTDSTPMFEYENLEDNSKEWTSLFDNDIPVTT | | | |
| | | regulatory\|pro- | DDVSLADKAIESTEEVSLVPSNLEVSTTSFLPT | | | |
| | | tein) | PVLEDAKLTQTRKVKKPNSVVKKSHHVGKDDES | | | |
| | | | RLDHLGVVAYNRKQRSIPLSPIVPESSDPAALK | | | |
| | | | RARNTEAARRSRARKLQRMKQLEDKVEELLSKN | | | |
| | | | YHLENEVARLKKLVGER | | | |
| SWISS_PR | 060282 | Kinesin heavy | MADPAECSIKVMCRFRPLNEAEILRGDKFIPKF (SEQ ID NO: 105) | | | |
| OT: | O95079 | chain isoform | KGDETVVIGQGKPYVFDRVLPPNTTQEQVYNAC | | | |
| KF5C_HUM | | 5C (Kinesin | AKQIVKDVLEGYNGTIFAYGQTSSGKTHTMEGK | | | |
| AN | | heavy chain | LHDPQLMGIIPRIAHDIFDHIYSMDENLEFHIK | | | |
| | | neuron-specfic\| | VSYFEIYLDKIRDLLDVSKTNLAVHEDKNRVPY | | | |
| | | 2) | VKGCTERFVSSPEEVMDVIDEGKANRHVAVTNM | | | |
| | | | NEHSSRSHSIFLINIKQENVETEKKLSGKLYLV | | | |
| | | | DLAGSEKVSKTGAEGAVLDEAKNINTNKSLSALGN | | | |
| | | | VISALAEGTKTHVPYRDSKMTRILQDSLGGNCR | | | |
| | | | TTTVICCSPSVFNEAETKSTLMFGQRAKTIKNT | | | |
| | | | VSVNLELTAEEWKKKYEKEKEKNKTLKNVIQHL | | | |
| | | | EMELNRWRNGEAVPEDEQISAKDQKNLEPCDNT | | | |
| | | | PIIDNIAPVVAGISTEEKEKYDEEISSLYRQLD | | | |
| | | | DKDDEINQQSQLAEKLKQQMLDQDELLASTRRD | | | |
| | | | YEKIQEELTRLQIENEAAKDEVKEVLQALEELA | | | |
| | | | VNYDQKSQEVEDKTRANEQLTDELAQKTTTLTT | | | |
| | | | TQRELSQLQELSNHQKKRATEILNLLLKDLGEI | | | |
| | | | QGIIGTNDVKTLADVNGVIEEEFTMARLYISKM | | | |
| | | | KSEVKSLVNRSKQLESAQMDSNRKMNASERELA | | | |
| | | | ACQLLISQHEAKIKSLTDYMQNMEQKRRQLEES | | | |
| | | | QDSLSEELAKLRAQEKMHEVSFQDKEKEHLTRL | | | |
| | | | QDAEEMKKALEQQMESHREAHQKQLSRLRDEIE | | | |
| | | | EKQKIIDEIRDLNQKLQLEQEKLSSDYNKLKIE | | | |
| | | | DQEREMKLEKLLLLNDKREQAREDLKGLEETVS | | | |
| | | | RELQTLHNLRKLFVQDLTTRVKKSVELDNDDGG | | | |
| | | | GSAAQKQKISFLENNLEQLTKVHKQLVRDNADL | | | |
| | | | RCELPKLEKRLRATAERVKALESALKEAKENAM | | | |
| | | | RDRKRYQQEVDRIKEAVRAKNMARRAHSAQIAK | | | |
| | | | PIRPQHYPASSPTAVHAIRGGGGSSSNSTHYQK | | | |
| SWISS_PR | P28738 | Kinesin heavy | MADPAECSIKVMCRFRPLNEAEILRGDKFIPKF | (SEQ ID NO:106) | | |
| OT: | Q9Z2F8 | chain isoform | KGEETVVIGQGKPYVFDRVLPPNTTQEQVYNAC | | | |
| KF5C_MOU | | 5C (Kinesin | AKQIVKDVLEGYNGTIFAYQQTSSGKTHTMEGK | | | |
| SE | | heavy chain | LHDPQLMGTIPRIAHDIFDHILYSMDENLEFHIK | | | |
| | | neuron-specific | VSYFEIYLDKIRDLLDVSKTNLAVHEDKNRVPY | | | |
| | | specific\|2) | VKGCTERFVSSPEEVMDVIDEGKANRHVAVTNM | | | |
| | | | NEHSSRSHSIFLINIKQENVETEKKLSGKLYLV | | | |
| | | | DLAGSEKVSKTGAEGAVLDEAKNINKSLSALGN | | | |
| | | | VISALAEGTKTHVPYRDSKMTRILQDSLGGNCR | | | |
| | | | TTIVILCCSPSVFNEAETKSTLMFGQRAKTIKNT | | | |
| | | | VSVNLELTAEEWKKKYEKEKEKNKALKSVLQHL | | | |
| | | | EMELNRWRNGEAVPEDEQTSAKDHKSLEPCDNT | | | |
| | | | PIIDNITPVVDGISAEKEKYDEEITSLYRQLDD | | | |
| | | | KDDEINQQSQLAEKLKQQMLDQDELLASTRRDY | | | |
| | | | EKIQEELTRLQIENEAAKDEVKEVLQALEELAV | | | |
| | | | NYDQKSQEVEDKTRANEQLTDELAQKTTTLTTT | | | |
| | | | QRELSQLQELSNHQKKRATEILNLLLKDLGEIG | | | |
| | | | GIIGTNDVKTLADVNGVIEEEFTMARLYISKMK | | | |
| | | | SEVKSLVNRSKQLESAQMDSNRKMNASERELAA | | | |
| | | | CQLLISQHEAKIKSLTDYMQNMEQKRRQLEESQ | | | |
| | | | DSLSEELAKLRAQEKMHEVSFQDKEKEHLTRLQ | | | |
| | | | DAEEVKKALEQQMESHREALHQKQLSRLRDEIEE | | | |
| | | | KQRIIDEIRDLNQKLQLEQERLSSDYNKLKIED | | | |
| | | | QEREVKLEKLLLLNDKREQAREDLKGLEETVSI | | | |
| | | | ELQTLHNLRKLFVQDLTTRVKKSVELDSDDGGG | | | |
| | | | SAAQKQKISFLENNLEQLTKVHKQLVRDNADLR | | | |
| | | | CELPKLEKRLRATAERVKALESALKEAKENAMR | | | |
| | | | DRKRYQQEVDRIKEAVRAKNMARRAHSAQIAKP | | | |
| | | | IRPGHYPASSPTAVHAVRGQGGQSSNSTHYQK | | | |
| SWISS_PR | P34540 | Kinesin heavy | MEPRTDGAECGVQVFCRIRPLNKTEEKNADRFL | (SEQ ID NO:107) | | |
| OT: | | chain | PKFPSEDSISLGGKVYVFDKVFKPNTTQEQVYK | | | |
| KINH_CAE | | | GAAYHIVQDVLSQYNGTVFAYGQTSSGKTHTME | | | |
| EL | | | GVIGDNGLSGIIPRIVADIFNHIYSMDENLQFH | | | |
| | | | IKVSYYETYNEKIRDLLDPEKVNLSIHEDKNRV | | | |
| | | | PYVKGATERFVGGPDEVLQAIEDGKSNRMVAVT | | | |
| | | | NMNEHSSRSHSVFLITVKQEHQTTKKQLTGKLY | | | |
| | | | LVDLAGSEKVSKTGAQGTVLEEAKNINKSLTAL | | | |
| | | | GIVISALAEGTKSHVPYRDSKLTRILQESLGGN | | | |
| | | | SRTTVIICASPSHFNEAETKSTLLFGARAKTIK | | | |
| | | | NVVQINEELTAEEWKRRYEKEKEKNTRLAALLQ | | | |
| | | | AAALELSRWRAGESVSEVEWVNLSDSAQMAVSE | | | |
| | | | VSGGSTPLMERSTAPAPPMLTSTTGPITDEEKK | | | |
| | | | KYEEERVKLYQQLDEKDDEIQKVSQELEKLRQQ | | | |
| | | | VLLQEEALGTMRENEELIREENNRFQKEAEDKQ | | | |
| | | | QEGKEMMTALEEIAVNLDVRQAECEKLKRELEV | | | |
| | | | VQEDNQSLEDRMNQATSLLNAHLDECGPKIRHF | | | |
| | | | KEGIYNVIREFNIADIASQNDQLPDHDLLNHVR | | | |
| | | | IGVSKLFSEYSAAKESSTAAEHDAEAKLAADVA | | | |
| | | | RVESGQDAGRMKQLLVKDQAAKEIKPLTDRVNM | | | |
| | | | ELTTLKNLKKEFMRVLVARCQANQDTEGEDSLS | | | |
| | | | GPAQKQRIQFLENNLDKLTKVHKQLVRDNADLR | | | |
| | | | VELPKMEARLRGREDRIKILETALRDSKQRSQA | | | |
| | | | ERKKYQQEVERIKEAVRQRNMRRMNAPQIVKPI | | | |
| | | | RPGQVYTSPSAGMSQGAPNGSNA | | | |
| SWISS_PR | P17210 | Kinesin heavy | MSAEREIPAEDSIKVVCRFRPLNDSEEKAGSKF | (SEQ ID NO:108) | | |
| OT: | QSV7L9 | chain | VVKFPNNVEENCISIAGKVYLFDKVFKPNASQE | | | |
| KINH_DRO | | KVYNKAAKSIVTDVLAGYNGTIFAYGQTSSGKT | | | | |
| ME | | | HTMEQVIGDSVKQGIIPRIVNDIFNHIYAMEVN | | | |
| | | | LEFHIKVSYYEIYMDKIRDLLDVSKVNLSVHED | | | |
| | | | KNRVPYVKGATERFVSSPEDVFEVIEEGKSNRH | | | |
| | | | IAVTNMNEHSSRSHSVFLINVKQENLENQKKLS | | | |
| | | | GKLYLVDLAGSEKVSKTGAEGTVLDEAKNINKS | | | |
| | | | LSALGNVISALADGNKTHIPYRDSKLTRILQES | | | |
| | | | LGGNARTTIVICCSPASFNESETKSTLDFGRRA | | | |
| | | | KTVKNVVCVNEELTAEEWKRRYEKEKEKNARLK | | | |
| | | | GKVEKLEIELARWRAGETVKAEEQINMEDLMEA | | | |
| | | | STPNLEVEAAQTAAAEAALAAQRTALANMSASV | | | |
| | | | AVNEQARLATECERLYQQLDDKDEEINQQSQYA | | | |
| | | | EQLKEQVMEQEELIANARREYETLQSEMARIQQ | | | |
| | | | ENESAKEEVKEVLQALEELAVNYDQKSQEIDNK | | | |
| | | | NKDIDALNEELQQKQSVFNAASTELQQLKDMSS | | | |
| | | | HQKKRITEMLTNLLRDLGEVGQAIAPGESSIDL | | | |
| | | | KMSALAGTDASKVEEDFTMARLFISKMKTEAKN | | | |
| | | | IAQRCSNMETQQADSNKKISEYEKDLQEYRLLI | | | |
| | | | SQHEARMKSLQESMREAENKKRTLEEQIDSLRE | | | |
| | | | ECAKLKAAEHVSAVNAEEKQRAEELRSMFDSQM | | | |
| | | | DELREAHTRQVSELRDEIAAKQHEMDEMKDVHQ | | | |
| | | | KLLLAHQQMTADYEKVRQEDAEKSSELQNIILT | | | |
| | | | NERREQARKDLKGLEDTVAKELQTLHNLRKLFV | | | |
| | | | QDLQQRIRKNVVNEESEEDGGSLAQKQKISFLE | | | |
| | | | NNLDQLTKVHKQLVRDNADLRCELPKLEKRLRC | | | |
| | | | TMERVKALETALKEAKEGAMRDRKRYQYEVDRI | | | |
| | | | KEAVRQKHLGRRGPQAQIAKPIRSGQGAIAIRG | | | |
| | | | GGAVGGPSPLAQVNPVNS | | | |
| SWISS_PR | P33176 | Kinesin heavy | MADLAECNIKVMCRFRPLNESEVNRGDKYIAKF | (SEQ ID NO:109) | | |
| OT: | | chain | QGEDTVVIASKPYAFDRVFQSSTSQEQVYNDCA | | | |
| KINH_HUM | (Ubiguitous | KKIVKDVLEGYNGTILFAYGQTSSGKTHTMEGKL | | | | |
| AN | | kinesin heavy | HDPEGMGIIPRIVQDIFNYIYSMDENLEFHIKV | | | |
| | | chain) (UKHC) | SYFEIYLDKIRDLLDVSKTNLSVHEDKNRVPYV | | | |
| | | | KGCTERFVCSPDEVMDTIDEGKSNRHVAVTNMN | | | |
| | | | EHSSRSHSIFLINVKQENTQTEQKLSGKLYLVD | | | |
| | | | LAGSEKVSKTGAEGAVLDEAKNINKSLSALGNV | | | |
| | | | ISALAEGSTYVPYRDSKMTRILQDSLGGNCRTT | | | |
| | | | IVICCSPSSYNESETKSTLLFGQRAKTIKNTVC | | | |
| | | | VNVELTAEQWKKKYEKEKEKNKILRNTIQWLEN | | | |
| | | | ELNRWRNGETVPIDEQFDKEKANLEAFTVDKDI | | | |
| | | | TLTNDKPATAIGVIGNFTDAERRKCEEEIAKLY | | | |
| | | | KQLDDKDEEINQQSQLVEKLKTQMLDQEELLAS | | | |
| | | | TRRDQDNMQAELNRLQAENDASKEEVKEVLQAL | | | |
| | | | EELAVNYDQKSQEVEDKTKEYELLSDELNQKSA | | | |
| | | | TLASIDAELQKLKEMTNHQKKRAAEMMASLLKD | | | |
| | | | LAEIGIAVGNNDVKQPEGTGMIDEEFTVARLYI | | | |
| | | | SKMKSEVKTMVKRCKQLESTQTESNKKMEENEK | | | |
| | | | ELAACQLRISQHEAKIKSLTEYLQNVEQKKRQL | | | |
| | | | EESVDALSEELVQLRAQEKVHEMEKEHLNKVQT | | | |
| | | | ANEVKQAVEQQIQSHRETHQKQISSLRDEVEAK | | | |
| | | | AKLITDLQDQNQKMMLEQERLRVEHEKLKATDQ | | | |
| | | | EKSRKLHELTVMQDRREQARQDLKGLEETVAKE | | | |
| | | | LQTLHNLRKLFVQDLATRVKKSAEIDSDDTGGS | | | |
| | | | AAQKQKISFLENNLEQLTKVHKQLVRDNADLRC | | | |
| | | | ELPKLEKRLPATAERVKALESALKEAKENASRD | | | |
| | | | RKRYQQEVDRIKEAVRSKNMARRGHSAQIAKPI | | | |
| | | | RPGQHPAASPTHPSAIRGGGAFVQNSQPVAVRG | | | |
| | | | GGGKQV | | | |
| SWISS_PR | P21613 | Kinesin heavy | MDVASECNIKVICRVRPLNEAEERAGSKFILKF | (SEQ ID NO:110) | | |
| OT: | | chain | PTDDSISIAGKVFVFDKVLKPNVSQEYVYNVCA | | | |
| KINH_LOL | | | KPIVADVLSGCNGTIFAYGQTSSGKTHTMEGVL | | | |
| PE | | | DKPSMHGIIPRIVQDIFNYTYGMDENLEFHIKI | | | |
| | | | SYYEIYLDKIRDLLDVTKTNLAVHEDKNRVPFV | | | |
| | | | KGATERFVSSPEEVMEVIDEGKNNRHVAVTNMN | | | |
| | | | EHSSRSHSVFLINVKQENVETQKKLSGKLYLVD | | | |
| | | | LAGSEKVSKTQAEGAVLDEAKNINKSLSALGNV | | | |
| | | | ISALADGNKSHVPYRDSKLTRILQESLGGNART | | | |
| | | | TMVICCSPASYNESETKSTLLFGQPAKTIKNVV | | | |
| | | | SVNEELTADEWKRRYEKEKERVTKLKATMAKLE | | | |
| | | | AELQRWRTGQAVSVEEQVDLKEDVPAESPATST | | | |
| | | | TSLAGGLIASMNEGDRTQLEEERLKIYQQLDDK | | | |
| | | | DDEINNQSQLIEKLKEQMMEQEDLIAQSRRDYE | | | |
| | | | NLQQDMSRIQADNESAKDEVKEVLQALEELAIVIN | | | |
| | | | YDQKSQEVEDKNKENENLSEELNQKLSTLNSLQ | | | |
| | | | NELDQQLKDSSMHHRKRVTDMMINLLKDLCDIGT | | | |
| | | | IVGGNAAETKPTAGSGEKIEEEFTVARLYISKM | | | |
| | | | KSEVKTLVSRNNQLENTQQDNFKKIETHEKDLS | | | |
| | | | NCKLLIQQHEAKMASLQEAIKDSENKKRMLEDN | | | |
| | | | VDSLNEEYAKLKAQEQMHLAALSEREKETSQAS | | | |
| | | | ETREVLEKQMEMHREQHQKQLQSLRDEISEKQA | | | |
| | | | TVDNLKDDNQRLSLALEKLQADYDKLKQEEVEK | | | |
| | | | AAKLADLSLQIDRREQAKQDLKGLEETVAKELQ | | | |
| | | | TLHNLRKLFVQDLQNKVKKSCSKTEEEDEDTGG | | | |
| | | | NAAQKQKISFLENNLEQLTKVHKQLVRDNADLR | | | |
| | | | CELPKLEKRLRATMERVKSLESALKDAKEGAMR | | | |
| | | | DRKRYQHEVDRIKEAVRQKNLARRGHAAQIAKP | | | |
| | | | IRPGQHQSVSPAQAAAIRGGGGLSQNGPMITST | | | |
| | | | PIRMAPESKA | | | |
| SWISS_PR | Q61768 | Kinesin heavy | MADPAECNIKVMCRFRPLNESEVNRGDKYVAKF | (SEQ ID NO:111) | | |
| OT: | O08711 | chain | QGEDTVVIASKPYAFDRVFQSSTSQEQVYNDCA | | | |
| KINH_MQU | Q61580 | (Ubiguitous | KKIVKDVLEGYNGTILFAYGQTSSGKTHTMEGKL | | | |
| SE | | kinesin heavy | HDPEGMGIIPRIVQDIFNYIYSMDENLEFHIKV | | | |
| | | chain) (UKHC) | SYFEIYLDKIRDLLDVSKTNLSVHEDKNRVPYV | | | |
| | | | KGCTERFVCSPDEVMDTIDEGKSNRHVAVTNMN | | | |
| | | | EHSSRSHSIFLINVKQENTQTEQKLSGKLYLVD | | | |
| | | | LAGSEKVSKTGAEGAVLDEAKNINKSLSALGNV | | | |
| | | | ISALAEGSTYVPYRDSKMTRILQDSLGGNCRTT | | | |
| | | | IVICCSPSSYNESETKSTLLFGQRAKTIKNTVC | | | |
| | | | VNVELTAEQWKKKYEKEKEKNKTLRNTIQWLEN | | | |
| | | | ELNRWRNGETVPIDEQFDKEKANLEAFTADKDI | | | |
| | | | AITSDKGAAAVGMAGSFTDAERRKCEEELAKLY | | | |
| | | | KQLDDKDEEINQQSQLVEKLKTQMLDQEELLAS | | | |
| | | | TRRDQDNMQAELNRLQAENDASKEEVKEVLQAL | | | |
| | | | EELAVNYDQKSQEVEDKTKEYELLTDEFNQKSA | | | |
| | | | TLASIDAELQKLKEMTNHQKKRAAEMMASLLKD | | | |
| | | | LAEIGIAVGNNDVKQPEGTGMTDEEFTVARLYI | | | |
| | | | SKMKSEVKTMVKRCKQLESTQTESNKKMEENEK | | | |
| | | | ELAACQLRISQHEAKIKSLTEYLQNDEQKKRQL | | | |
| | | | EESLDSLGEELVQLRAQEKVHEMEKEHLNKVQT | | | |
| | | | ANEVKQAVEQQIQSHRETHQKQISSLRDEVEAK | | | |
| | | | EKLITDLQDQNQKMVLETERLRVEHERLKATDQ | | | |
| | | | EKSRKLHELTVMQDRREQARQDLKGLEETVAKE | | | |
| | | | LQTLHNLRKLFVQDLATRVKKSAEVDSDDTGGS | | | |
| | | | AAQKQKISFLENNLEQLTKVHKQLVRDNADLRC | | | |
| | | | ELPKLEFRLRATAERVKALESALKEAKENASRD | | | |
| | | | RKRYQQEVDRIKEAVRSKNMARRGHSAQIAKPI | | | |
| | | | RPGQHPAASPTHPGTVRGGGSFVQNNQPVGLRG | | | |
| | | | GGGKQS | | | |
| SWISS_PR | P48467 | Kinesin heavy | MSSSANSIKVVARFRPQNRVEIESGGQPTVTFQ | (SEQ ID NO:112) | | |
| OT: | | chain | QPDTCTVDSKEAQGSFTFDRVFDMSCKQSDIFD | | | |
| KINH_NEU | | | FSIKPTVDDILNGYNGTVFAYGQTGAGKSYTMM | | | |
| CR | | | GTSIDDPDGRGVIPRIVEQIFTSILLSSAANIEY | | | |
| | | | TVRVSYMEIYMERIRDLLAPQNDNLPVHEEKNR | | | |
| | | | GVYVKGLLETYVSSVQEVYEVMRRGGNARAVAA | | | |
| | | | TNMNQESSRSHSIFVITITQKNVETGSAKSGQL | | | |
| | | | FLVDLAGSEKVGKTGASGQTLEEAKKINKSLSA | | | |
| | | | LGMVINALTDGKSSHVPYRDSKLTRILQESLGG | | | |
| | | | NSRTTLIINCSPSSYNDAETLSTLRFGMRAKSI | | | |
| | | | KNKAKVNAELSPAELKQMLAKAKTQITSFENYI | | | |
| | | | VNLESEVQVWRGQETVPKEKWVPPLELAITPSK | | | |
| | | | SASTTARPSTPSRLLPESRAETPAISDRAGTPS | | | |
| | | | LPLDKDEREEFLRRENELQDQIAEKESIAAAAE | | | |
| | | | RQLRETKEELIALKDHDSKLGKENERLISESNE | | | |
| | | | FKMQLERLAFENKEAQITIDGLKDANSELTAEL | | | |
| | | | DEVKQQMLDMKMSAKETSAVLDEKEKKKAEKMA | | | |
| | | | KMMAGFDLSGDVFSDNERAVADAIAQLDALFEI | | | |
| | | | SSAGDAIPPEDIKALREKLVETQGFVRQAELSS | | | |
| | | | FSAASSDAEARKRAELEARLEALQQEHEELLSR | | | |
| | | | NLTEADKEEVKALLAKSLSDKSAVQVELVEQLK | | | |
| | | | ADIALKNSETEHLKALVDDLQRRVKAGGAGVAM | | | |
| | | | ANGKTVQQQLAEFDVMKKSLMRDLQNRCERVVE | | | |
| | | | LEISLDETREQYNNVLRSSNNRAQQKKMAFLER | | | |
| | | | NLEQLTQVQRQLVEQNSALKKEVAIAERKLMAR | | | |
| | | | NERIQSLESLLQESQEKMAQANHKFEVQLAAVK | | | |
| | | | DRLEAAKAGSTRGLGTDAGLGGFSIGSRIAKPL | | | |
| | | | RGGGDAVAGATATNPTIATLQQNPPENKRSSWF | | | |
| | | | FQKS | | | |
| SWISS_PR P35978 | Kinesin heavy | MADPAECNIKVVCRVRPMNATEQNTSHICTKFI | (SEQ ID NO:113) | | | |
| OT: | | chain | SEEQVQIGGKLNMFDRIFKPNTTQEEVYNKAAR | | | |
| KINH_STR | | | QILVKDVLDGYNGTIFAYGQTSSGKTFTMEGVMG | | | |
| PU | | | NPQYMGIIPRIVQDIFNHIYQMDESLEFHIKVS | | | |
| | | | YFEIYMDRIRDLLDVSKTNLSVHEDKNRVPFVK | | | |
| | | | GATERFASSPEEVMDVIEEGKSNRHIAVTNMNE | | | |
| | | | HSSRSHSIFLIQVKQENMETKKKLSGKLYLVDL | | | |
| | | | AGSEKVSKTGAEGTVLDEAKNINKSLSALGNVI | | | |
| | | | SALADGKKSHIPYRDSKMTRILQESLCGNARTT | | | |
| | | | IVICCSPSSFNESESKSTLMFGQRAKTIKNTVT | | | |
| | | | VNMELTAEEWRNRYEKEKEKNGRLKAQLLILEN | | | |
| | | | ELQRWRAGESVPVKEQGNKNDEILKEMMKPKQM | | | |
| | | | TVHVSEEEKNKWEEEKVKLYEQLDEKDSEIDNQ | | | |
| | | | SRLTEKLKQQMLEQEELLSSMQRDYELLQSQMG | | | |
| | | | RLEAENAAAKEEAKEVLQALEEMAVNYDEKSKE | | | |
| | | | VEDKNRMNETLSEEVNEKMTALHTTSTELQKLQ | | | |
| | | | ELEQHQRRRITEMMASLLKDLGEIGTALQGNAA | | | |
| | | | DMKPNVENIEKVDEEFTMARLFVSKMKTEVKTM | | | |
| | | | SQRCKILEASNAENETKIRTSEDELDSCRMTIQ | | | |
| | | | QHEAKMKSLSENIRETEGKKRHLEDSLDMLNEE | | | |
| | | | IVKLRAAEEIRLTDQEDKKREEEDKMQSATEMQ | | | |
| | | | ASMSEQMESHRDAHQKQLANLRTEINEKEHQME | | | |
| | | | ELKDVNQRMTLQHEKLQLDYEKLKIEEAEKAAK | | | |
| | | | LRELSQQFDRREQAKQDLKGLEETVAKELQTLH | | | |
| | | | NLRKLFVSDLQNRVKKALEGGDRDDDSGGSQAQ | | | |
| | | | KQKISFLENNLEQLTKVHKQLVRDNADLRCELP | | | |
| | | | KLERRLRATSERVKALEMSLKETKEGAMRDRKR | | | |
| | | | YQQEVDRIREAVRQRNFAKRGSSAQIAKAIRAG | | | |
| | | | HPPPSPGGSTGIRGGGYSGIRGGGSPVIRPPSH | | | |
| | | | GSPEPISHNNSFEKSLNPNDAENMEKKANKRLP | | | |
| | | | KLPPGGNKLTESDIAANKARSKARNNTPGKAPL | | | |
| | | | TTSGEQGS | | | |
| SWISS_PR | 043093 | Kinesin heavy | MSGNNIKVVCRFRPQNSLEIREGGTPIIDIDPE | (SEQ ID NO:114) | | |
| OT: KINH_ | | chain (Synkin) | GTQLELKGKEFKGNFNFDKVFGMNTAQKDVFDY | | | |
| SYNRA | | | SIKTIVDDVTAGYNGTVFAYGQTGSGKTFTMMG | | | |
| | | | ADIDDEKTKGIIPRIVEQIFDSIMASPSNLEFT | | | |
| | | | VKVSYMEIYMEKVRDLLNPSSENLPIHEDKTKG | | | |
| | | | VYVKGLLEVYVGSTDEVYEVMRRGSNNRVVAYT | | | |
| | | | NMNAESSRSHSIVMFTITQKNVDTGAAKSGKLY | | | |
| | | | LVDLAGSEKVGKTGASGQTLEEAKKINKSLTAL | | | |
| | | | GMVINALTDGKSSHVPYRDSKLTRILQESLGGN | | | |
| | | | SRTTLIINCSPSSYNEAETLSTLRFGAPAKSIK | | | |
| | | | NKAKVNADLSPAELKALLKKVKSEAVTYQTYIA | | | |
| | | | ALEGEVNXTWRTGGTVPEGKWVTMDKVSKGDFAG | | | |
| | | | LPPAPGFKSPVSDEGSRPATPVPTLEKDEREEF | | | |
| | | | IKRENELMDQISEKETELTNREKLLESLREEMG | | | |
| | | | YYKEQEQSVTKENQQMTSELSELRLQLQKVSYE | | | |
| | | | SKENAITVDSLKEANQDLMAELEELKKNLSEMR | | | |
| | | | QAHKDATDSDKEKRKAEKMAQMMSGFDPSGILN | | | |
| | | | DKERQIRNALSKLDGEQQQTLTVEDLVSLRREL | | | |
| | | | AESKMLVEQHTKTISDLSADKDANEAKKIELEG | | | |
| | | | RLGALEKEYEELLDKTIAEEEANMQNADVDNLS | | | |
| | | | ALKTKLEAQYAEKKEVQQKEIDDLKRELDRKQS | | | |
| | | | GHEKLSAAMTDLRAANDQLQAALSEQPFQAPQD | | | |
| | | | NSDMTEKEKDIERTRKSMAQQLADFEVMKKALM | | | |
| | | | RDLQNRCEKVVELEMSLDETREQYNNVLRASNN | | | |
| | | | KAQQKKMAFLERNLEQLTNVQKQLVEQNAS LKK | | | |
| | | | EVALAERKLIARNERIQSLETLLHNAQDKLLNQ | | | |
| | | | NKKFEQQLATVRERLEQARSQKSQNSLAALNFS | | | |
| | | | RIAKPLRGNGAAIDNGSDDGSLPTSPTDKRDKR | | | |
| | | | SSWMPGFMNSR | | | |
| SWISS_PR | Q12840 | Neuronal | MAETNNECSIKVLCRFRPLNQAEILRGDKFIPI | (SEQ ID NO:115) | | |
| OT: | | kinesin heavy | FQGDDSVVIGGKPYVFDRVFPPNTTQEQVYHAC | | | |
| KINN_HUM | | chain (NKHC) | AMQIVKDVLAQYNGTIFAYGQTSSGKTHTMEGK | | | |
| AN | | (Kinesin heavy | LHDPQLMGIIPRIARDIFNHIYSMDENLEFHIK | | | |
| | | chain isoform | VSYFEIYLDKIRDLLDVTKTNLSVHEDKNRVPF | | | |
| | | 5A)\|(Kinesin | VKGCTERFVSSPEEILDVIDEGKSNRHVAVTNM | | | |
| | | heavy chain | NEHSSRSHSIFLINIKQENMETEQKLSGKLYLV | | | |
| | | neuron-specific | DLAGSEKVSKTGAEGAVLDEAKNINKSLSALGN | | | |
| | | 1) | VISALAEGTKSYVPYRDSKMTRILQDSLGGNCR | | | |
| | | | TTMFICCSPSSYNDAETKSTLMFGQRAKTIKNT | | | |
| | | | ASVNLELTAEQWKKKYEKEKEKTKAQKETIAKL | | | |
| | | | EAELSRWRNGENVPETERLAGEEAALGAELCEE | | | |
| | | | TPVNDNSSIVVRIAPEERQKYEEEIRRLYKQLD | | | |
| | | | DKDDEINQQSQLIEKLKQQMLDQEELLVSTRGD | | | |
| | | | NEKVQRELSHLQSENDAAKDEVKEVLQALEELA | | | |
| | | | VNYDQKSQEVEEKSQQNQLLVDELSQKVATMLS | | | |
| | | | LESELQRLQEVSGHQRKRIAEVLNGLMKDLSEF | | | |
| | | | SVIVGNGEIKLPVEISQAIEEEFTVARLYISKT | | | |
| | | | KSEVKSVVKRCRQLENLQVECHRKMEVTGRELS | | | |
| | | | SCQLLISQHEAKIRSLTEYMQSVELKKRHLEES | | | |
| | | | YDSLSDELAKLQAQETVHEVALKDKEPDTQDAD | | | |
| | | | EVKKALELQMESHREAHHRQLARLRDEINEKQK | | | |
| | | | TIDELKDLNQKLQLELEKLQADYEKLKSEEHEK | | | |
| | | | STKLQELTFLYERHEQSKQDLKGLEETVARELQ | | | |
| | | | TLHNLRKLFVQDVTTRVKKSAEMEPEDSGGIHS | | | |
| | | | QKQKISFLENNLEQLTKVHKQLVRDNADLRCEL | | | |
| | | | PKLEKRLRATAERVKALEGALKEAKEGAMKDKR | | | |
| | | | RYQQEVDRIKEAVRYKSSGKRAHSAQIAKPVRP | | | |
| | | | GHYPASSPTNPYGTRSPECISYTNSLFQNYQNL | | | |
| | | | YLQATPSSTSDMYFANSCTSSGATSSGGPLASY | | | |
| | | | QKANMDNGNATDINDNRSDLPCGYEAEDQAKLF | | | |
| | | | PLHQETAAS | | | |
| SWISS_PR | P33175 | Neuronal | MAETNNECSIKVLCRFRPLNQAEILRGDKFTPI | (SEQ ID NO:116) | | |
| OT:KINN_ | Q9Z2F9 | kinesin heavy | FQGDDSVIIGGKPYVFDRVFPPNTTQEQVYHAC | | | |
| MOUSE | | chain (NKHC) | AMQIVKDVLAQYNGTIFAYGQTSSGKTHTMEGK | | | |
| | | (Kinesin heavy | LHDPQLMGIIPRIARDIFNHIYSMDENLEFHIK | | | |
| | | chain isoform | VSYFEIYLDKIRDLLDVTKTNLSVHEDKNRVPF | | | |
| | | 5A) | VKGCTERFVSSPEEILDVIDEGKSNRHVAVTNM | | | |
| | | (Kinesin heavy | NEHSSRSHSIFLINIKQENVETEQKLSGKLYLV | | | |
| | | chain | DLAGSEKVSKTGAEGAVLDEAKNINKSLSALGN | | | |
| | | neuron-specific | VISALAEGTKSYVPYRDTKMTRILQDSLGGNCR | | | |
| | | 1) | TTMFICCSPSSYNDAETKSTLMFGQRAKTIKNT | | | |
| | | | ASVNLELTAEQWKKKYEKEKEKTKAQKETIANV | | | |
| | | | EAELSRWRNGENVPETERLAGEDSALGAELCEE | | | |
| | | | TPVNDNSSIVVRTAPEERQKYEEEIRRLYKQLD | | | |
| | | | DKDDETNQQSQLIEKLKQQMLDQEELLVSTRGD | | | |
| | | | NEKVQRELSHLQSENDAAKDEVKEVLQALEELA | | | |
| | | | VNYDQKSQEVEEKSQQNQLLVDELSQKVATMLS | | | |
| | | | LESELQRLQEVSGHQRKRIAEVLNGLMRDLSEF | | | |
| | | | SVIVGNGEIKLPVEISGAIEEEFTVARLYISKI | | | |
| | | | KSEVKSVVKRCRQLENLQVECHRKMEVTGRELS | | | |
| | | | SCQLLISQHEAKIRSLTEYMQTVELKKRHLEES | | | |
| | | | YDSLSDELARLQAHETVHEVALKDKEPDTQDAE | | | |
| | | | EVKKALELQMENHREAHHRQLARLRDEINEKQK | | | |
| | | | TIDELKDLNQKLQLELEKLQADYERLKNEENEK | | | |
| | | | SAKLQELTFLYERHEQSKQDLKGLEETVARELQ | | | |
| | | | TLHNLRKLFVQDVTTRVKKSAEMEPEDSGGIHS | | | |
| | | | QKQKISFLENNLEQLTKVHKQLVRDNADLRCEL | | | |
| | | | PKLEKRLRATAERVKALEGALKEAKEGAMKDKR | | | |
| | | | RYQQEVDRIKEAVRYKSSGKRGHSAQIAKPVRP | | | |
| | | | QHYPASSPTNPYGTRSPECISYTNNLFQNYQNL | | | |
| | | | HLQAAPSSTSDMYFASSGRTSVAPLASYQKANM | | | |
| | | | DNGNATDINDNRSDLPCGYEAEDQAKLFPLHQE | | | |
| | | | TAAS | | | |
| SWISS_PR | P28742 | Kinesin-like | MARSSLPNRRTAQFEANKRRTIAHAPSPSLSNG | (SEQ ID NO:117) | | |
| OT: | | protein KIP1 | MHTLTPPTCNNGAATSDSNIRVYVRCRSRNKRE | | | |
| KIP1_YEA | | | TEEKSSVVTSTLCPQGKETTLSNGSHQSYSSSK | | | |
| ST | | | KTYQFDQVFGAESDQETVFNATAKNYTKEMLHG | | | |
| | | | YNCTTFAYGQTGTGKTYTMSGDINILGDVQSTD | | | |
| | | | NLLLGEHAGIIPRVLVDLFKELSSLNKEYSVKT | | | |
| | | | SFLELYNENLKDLLSDSEDDDPAVNDPKRQTRI | | | |
| | | | FDNNNNNSSIMVKGMQEIFINSAHEGLNLLMQG | | | |
| | | | SLKRKVAATKCNDLSSRSHTVFTITTNIVEQDS | | | |
| | | | KDHGQNKNFVKIGKLNLVDLAGSENINRSGAEN | | | |
| | | | KRAQEAGLINKSLLTLGRVINALVDHSNHIPYR | | | |
| | | | ESKLTRLLQDSLGQMTKTCITATISPAKISMEE | | | |
| | | | TASTLEYATRAKSIKNTPQVNQSLSKDTCLKDY | | | |
| | | | IQEIEKLRNDLKNSRNKQGIFITQDQLDLYESN | | | |
| | | | SILIDEQNLKIHNLREQIKKFKENYLNQLDINN | | | |
| | | | LLQSEKEKLIAIIQNFNVDFSNFYSEIQKIHHT | | | |
| | | | NLELMNEVTQQRDFSLENSQKQYNTNQNMQLKI | | | |
| | | | SQQVLQTLNTLQGSLNNYNSKCSEVIKGVTEEL | | | |
| | | | TRNVNTHKAKHDSTLKSLLNITTNLLMNQMNEL | | | |
| | | | VRSISTSLEIFQSDSTSHYRKDLNEIYQSHQQF | | | |
| | | | LKNLQNDTKSCLDSIGSSILTSTNEISQNCTTN | | | |
| | | | LNSMNVLIENQQSGSSKLIKEQDLEIKKLKNDL | | | |
| | | | INERRISNQFNQQLAEMKRYFQDHVSRTRSEFH | | | |
| | | | DELNKCTDNLKDKQSKLDQDIWQKTASTFNETD | | | |
| | | | IVVNKIHSDSIASLAHNAENTLKTVSQNNESFT | | | |
| | | | NDLISLSRGMNMDISSKLRSLPINEFLNKISQT | | | |
| | | | ICETCGDDNTIASNPVLTSIKKFQNTICSDIAL | | | |
| | | | TNEKTMSLIDEIQSQTETISNENNTNLTAINEN | | | |
| | | | FNSLCNFTLTDYDENIMQTSKTQDEVLSEHCEK | | | |
| | | | LQSLKILGMDIFTAHSIEKPLHEHTRPEASVIK | | | |
| | | | ALPLLDYPKQFQIYRDAENKSKDDTSNSRTCIP | | | |
| | | | NLSTNENFPLSQFSPKTPVPVPDQPLPKVLIPK | | | |
| | | | SINSAKSNRSKTLPNTEGTGRESQNNLKRRFTT | | | |
| | | | EPTLKGEETENNDTLQNKKLHQ | | | |
| SWISS_PR | P28743 | Kinesin-like | MIQKMSPSLRRPSTRSSSGSSNIPQSPSVRSTS | (SEQ ID NO:118) | | |
| OT: | | protein KIP2 | SFSNLTRNSIRSTSNSGSQSISASSTRSNSPLR | | | |
| KIP2_YEA | | | SVSAKSDPFLHPGRIRIRRSDSINNNSRKNDTY | | | |
| ST | | | TQSITVTIRPKPRSVGTSRDHVGLKSPRYSQPR | | | |
| | | | SNSHHGSNTFVRDPWFITNDKTIVHEEIGEFKF | | | |
| | | | DHVFASHCTNLEVYERTSKPMIDKLLMQFNATI | | | |
| | | | FAYGMTGSGKTFTMSGNEQELGLIPLSVSYLFT | | | |
| | | | NIMEQSMNGDKKFDVIISYLETYNERIYDLLES | | | |
| | | | GLEESGSRISTPSRLYMSKSNSNGLGVELKIRD | | | |
| | | | DSQYGVKVIGLTERRCESSEELLRWIAVGDKSR | | | |
| | | | KIGETDYNARSSRSHAIVLIRLTSTNVKNGTSR | | | |
| | | | SSTLSLCDLAGSERATGQQERRKEGSFINKSLL | | | |
| | | | ALGTVISKLSADKMNSVGSNIPSPSASGSSSSS | | | |
| | | | QNATNNGTSPSNHIPYRDSKLTRLLQPALSGDS | | | |
| | | | IVTTICTVDTRNDAAAETMNTLRFASRAKNVAL | | | |
| | | | HVSKKSIISNGNNDGDKDRTIELLRRQLEEQRR | | | |
| | | | MISELKNRSNIGEPLTKSSNESTYKDIKATQND | | | |
| | | | GDPNLALMRAENRVLKYKLENCEKLLDKDVVDL | | | |
| | | | QDSEIMEIVEMLPFEVGTLLETKFQGLESQIRQ | | | |
| | | | YRKYTQKLEDKIMALEKSGHTAMSLTQCDGTEV | | | |
| | | | IELQKMLERKDKMIEALQSAKRLRDRALKPLIN | | | |
| | | | TQQSPHPVVDNDK | | | |
| SWISS_PR | P46822 | Kinesin light | MSNMSQDDVTTGLRTVQQGLEALREEHSTISNT | (SEQ ID NO:119) | | |
| OT: | Q18088 | chain (KLC) | LETSVKGVKEDEAPLPKQKLSQINDNLDKLVCG | | | |
| KLC_CAEE | | | VDETSLMLMVFQLTQQMDAQHQKYQAQRRRLCQ | | | |
| L | | | ENAWLRDELSSTQIKLQQSEQMVAQLEEENKHL | | | |
| | | | KYMASIKQLDDGTQSDTKTSVDVGPQPVTNETL | | | |
| | | | QELGFGPEDEEDMNASQFNQPTPANQMAASANV | | | |
| | | | GYEIPARLRTLHNLVIQYASQGRYEVAVPLCKQ | | | |
| | | | ALEDLEKTSGHDHPDVATMLNILALVYRDQNKY | | | |
| | | | KEAANLLNEALSIREKCLGESHPAVAATLNNLA | | | |
| | | | VLFGKRGKFKDAEPLCKRALEIREKVLGDDHPD | | | |
| | | | VAKQLNNLALLCQNQGKYEEVEKYYKRALEIYE | | | |
| | | | SKLGPDDPNVAKTKNNLSSAYLKQGKYKEAEEL | | | |
| | | | YKQILTRAHEREFGQISGENKPIWQIAEEREEN | | | |
| | | | KHKGEGATANEQAGWAKAAKXTDSPTVTTTLKNL | | | |
| | | | GALYRRQGKYEAAETLEDVALRAKKQHEPLRSQ | | | |
| | | | ANGGIDEMSQSMMASTIQGSRNSMTTSTSQTGL | | | |
| | | | KNKLMNALGFNS | | | |
| SWISS_PR | P46824 | Kinesin light | MTQMSQDEIITNTKTVLQGLEALRVEHVSIMNQ | (SEQ ID NO:120) | | |
| OT: | Q9VU05 | chain (KLC) | IAEVQKDNEKSDMLRKNIENILELGLSEAQVMMA | | | |
| KLC_DROM | | | LTSHLQNEAEKHKLKTQVRRLHQENAWLRDEL | | | |
| E | | | ANTQQKFQASEQLVAQLEEEKKHLEFMASVKKY | | | |
| | | | DENQEQDDACDKSRTDPVVELFPDEENEDRHNM | | | |
| | | | SPTPPSQFANQTSQYEIPARLRTLHNLVIQYAS | | | |
| | | | QGRYEVAVPLCKQALEDLERTSGHDHPDVATML | | | |
| | | | NILALVYRDQNKYKEAANLLNDALSIRGKTLGE | | | |
| | | | NHPAVAATLNNLAVLYQKRGKYKDAEPLCKRAL | | | |
| | | | EIREKVLGKDHPDVAKQLNNLALLCQNQGKYDE | | | |
| | | | VEKYYQRALDIYESKLGPDDPNVAKTKNNLAGC | | | |
| | | | YLKQGRYTEAEILYKQVLTRAHEREFGAIDSKN | | | |
| | | | KPIWQVAEEREEHKFDNRENTPYQEYQQWHKAA | | | |
| | | | KVDSPTVTTTLKNLGALYRRQQMFEAAETLEDC | | | |
| | | | ANRSKKEAYDLAKQTKLSQLLTSNEKRRSKAIK | | | |
| | | | EDLDFSEEKNAKP | | | |
| SWISS_PR | P46825 | Kinesin light | MEVTQTVKSYRIKKIEEIQKMTALSQEEIISNT | (SEQ ID NO:121) | | |
| OT: | | chain (KLC) | KTVIQGLDTLKNEHNQILNSLLTSMKTIRKENG | | | |
| KLC_LOLP | | | DTNLVEEKANILKKSVDSIELGLGEAQVMMALA | | | |
| E | | | NHLQHTEAEKQKLRAQVRRLCQENAWLRDELAN | | | |
| | | | TQQKLQMSEQKVATIEEEKKHLEFMNEMKKYDT | | | |
| | | | NEAQVNEEKESEQSSLDLGFPDDDDDGGQPEVL | | | |
| | | | SPTQPSAMAQAASGQCEIPARLRTLHNLVIQYA | | | |
| | | | SQGRYEVAVPLCKQALEDLEKTSGHDHPDVATM | | | |
| | | | LNILALVYRDQGKYKEAANLLNDALGIREKTLG | | | |
| | | | PDHPAVAATLNNLAVLYGKRGKYKDAEPLCKRA | | | |
| | | | LVIREKVLGKDHPDVAKQLNNLALLCQNQGKYE | | | |
| | | | EVERYYQRALEIYQKELGPDDPNVAKTKNNLAS | | | |
| | | | AYLKQGKYKQAEILYKEVLTRAHEKEFGKVDDD | | | |
| | | | NKPIWMQAEEREENKAKYKDGAPQPDYQSWLKA | | | |
| | | | VKVDSPTVTTTLKLNLGALYRRQQKYEAAETLEE | | | |
| | | | CALRSRKSALEVVRQTKISDVLGSDFSKGQSPK | | | |
| | | | DRKRSNSRDRNRRDSMDSVSYEKSQDGDEHEKS | | | |
| | | | KLHVGTSHKQ | | | |
| SWISS_PR | Q05090 | Kinesin light | MSGSKLSTPNNSGGGQGNLSQEQTTTGTREVIK | (SEQ ID NO:122) | | |
| OT: | Q05089 | chain (KLC) | GLEQLKNEHNDILNSLYQSLKMLKKDTPGDSNL | | | |
| KLC_STRP | Q05088 | | VEEKTDIIEKSLESLELGLGEAKVMMALGHHLN | | | |
| U | Q04801 | | MVEAEKQKLRAQVRRLVQENTWLRDELAATQQK | | | |
| | | | LQTSEQNIADLEVKYKHLEYMNSIKKYDEDRTP | | | |
| | | | DEEASSSDPLDLGFPEDDDGQQADESYPQPQTG | | | |
| | | | SGSVSAAAQGYEIPARLRTLHNLVIQYASQSRY | | | |
| | | | EVAVPLCKQALEDLEKTSGNDHPDVATMLNILA | | | |
| | | | LVYRDQNKYKEAGNLLHDALAIREKTLGPDHPA | | | |
| | | | VAATLNNLAVLYGKRGKYKEAEPLCKRALEIRE | | | |
| | | | KVLGKDHPDVAKQLNNLALLCQNQGKYEEVEWY | | | |
| | | | YQRALEIYEKKLGPDDPNVAKTKNNLAAAYLKQ | | | |
| | | | GKYKAAETLYKQVLTRAHEREFGLSADDKDNKP | | | |
| | | | IWMQAEEREEKGKFKDNAPYGDYGGWHKAAKVD | | | |
| | | | SRSRSSPTVTTTLKNLGALYRRQGKYDAAEILE | | | |
| | | | ECAMKSRRNALDMVRETKVRELLGQDLSTDVPR | | | |
| | | | SEAMAKERHHRRSSGTPRHGSTESVSYEKTDGS | | | |
| | | | REVSIGVAWKAKRKAKDRSRSIPAGYVEIPRSP | | | |
| | | | PHVLVENGDGKLRRSGSLSKLRASVRRSSTKLL | | | |
| | | | NKLKGRESDDDGGMKRASSMSVLPSRGNDESTP | | | |
| | | | APIQLSQRGRVQSHDNLSSRRQSGNF | | | |
| SWISS_PR | O42401 | Matrilin-3 | MRRALGTLGCCLALLLPLLPAARGVPHRHRRQP | (SEQ ID NO:123) | | |
| OT: | | precursor | LGSQLGRHGAADTACKURPLDLVFIIDSSRSVR | | | |
| MTN3_CHI | | | PEEFEKVKIFLSKMIDTLDVGERTTRVAVMNYA | | | |
| | | CK | STVKVEFPLRTYFDKASMKEAVSRIQPLSAGTM | | | |
| | | | TGLAIQAAMDEVFTEEMGTRPANFNIPKVVIIV | | | |
| | | | TDGRPQDQVENVAANARTAGIEIYAVGVQRADM | | | |
| | | | QSLRIMASEPLDEHVFYVETYGVIEKLTSKERE | | | |
| | | | TFCAANTCALGTHDCEQVCVSNDGSYLCDCYEG | | | |
| | | | YTLNPDKRTCSAVDVCAPGRHECDQICVSNNGS | | | |
| | | | YVCECFEGYTLNPDKKTCSAMDVCAPGRHDCAQ | | | |
| | | | VCRRNGGSYSCDCFEGFTLNPDKKTCSAVDVCA | | | |
| | | | PGRHDCEQVCVRDDLFYTCDCYQGYVLNPDKKT | | | |
| | | | CSRATTSSLVTDEEACKCEAIAALQDSVTSRLE | | | |
| | | | ALSTKLDEVSQKLQAYQDRQQVV | | | |
| SWISS_PR | O15232 | Matrilin-3 | MPRPAPARRLPGLLLLLWPLLLLPSAAPDPVAR | (SEQ ID NO:124) | | |
| OT: | | precursor | PGFRRLETRGPGGSPGRRPSPAAPDGAPASGTS | | | |
| MTN3_HUM | | | EPGRARGAGCKSRPLDLVFIIDSSRSVRPLEF | | | |
| AN | | | TKVKTFVSRIIDTLDIGPADTRVAVVNYASTVK | | | |
| | | | IEFQLQAYTDKQSLKQAVGRITPLSTGTMSGLA | | | |
| | | | IQTAMDEAFTVEAGAREPSSNIPKVAIIVTDGR | | | |
| | | | PQDQVNEVAARAQASGIELYAVGVDRADMASLK | | | |
| | | | MMASEPLEEHVFYVETYGVIEKLSSRFQETFCA | | | |
| | | | LDPCVLGTHQCQHVCISDGEGKHHCECSQGYTL | | | |
| | | | NADKKTCSALDRCALNTHGCEHICVNDRSGSYH | | | |
| | | | CECYEGYTLNEDRKTCSAQDKCALGTHGCQHIC | | | |
| | | | VNDRTGSHHCECYEGYTLNADKKTCSVRDKCAL | | | |
| | | | GSHGCQHICVSDGAASYHCDCYPGYTLNEDKKT | | | |
| | | | CSATEEARRLVSTEDACGCEATLAFQDKVSSYL | | | |
| | | | QRLNTKLDDILEKLKINEYGQIHR | | | |
| SWISS_PR | O35701 | Matrilin-3 | MLLSAPLRHLPGLLLLLWPLLLLPSLAAPGRLA | (SEQ ID NO:125) | | |
| OT: | Q9JHM0 | precursor | RASVRRLGTRVPGGSPGHLSALATSTRAPYSGG | | | |
| MTN3_MOU | | | RGAGVCKSRPLDLVFIIDSSRSVRPLEFTKVKT | | | |
| SE | | | FVSRIIDTLDIGATDTRVAVVNYASTVKIEFQL | | | |
| | | | NTYSDKQALKQAVARITPLSTGTMSGLALQTAN | | | |
| | | | EEAFTVEAGARGPMSNIPKVAIIVTDGRPQDQV | | | |
| | | | NEVAARARASGIELYAVGVDRADMESLKMMASK | | | |
| | | | PLEEHVFYVETYGVIEKLSARFQETFCALDQCM | | | |
| | | | LGTHQCQHVCVSDGDGKHHCECSQGYTLNADGK | | | |
| | | | TCSAIDKCALSTHGCEQICINDRNGSYHCECYG | | | |
| | | | GYALNADRRTCAALDKCASGTHGCQHICVNDGA | | | |
| | | | GSHHCECFEGYTLNADKKTCSVRNKCALGTHGC | | | |
| | | | QHICVSDGAVAYHCDCFPGYTLNDDKKTCSDIE | | | |
| | | | EARSLISIEDACGCGATLAFQEKVSSHLQKLNT | | | |
| | | | KLDNILKKLKVTEYGQVHR | | | |
| SWISS_PR | Q24167 | Similar protein | MVSLIDTIEAAAEKQKQSQAVVTNTSASSSSCS | (SEQ ID NO:126) | | |
| OT: | Q9VAA5 | | SSFSSSPPSSSVGSPSPGAPKTNLTASGKPKEK | | | |
| SIMA_DRO | | | RRNNEKRKEKSRDAARCRRSKETEIFMELSAAL | | | |
| ME | | | PLKTDDVNQLDKASVMRITIAFLKTREMLQFVP | | | |
| | | | SLRDCNDDIKQDIETAEDQQEVKPKLEVGTEDW | | | |
| | | | LNGAEARELLKQTMDGFLLVLSHEGDITYVSEN | | | |
| | | | VVEYLGITKIDTLGQQIWEYSHQCDHAEIKEAL | | | |
| | | | SLKRELAQKVKDEPQQNSGVSTHHRDLFVRLKC | | | |
| | | | TLTSRGRSINIKSASYKVIHITGHLVVNAKGER | | | |
| | | | LLMAIGRPIPHPSNIEIPLGTSTFLTKHSLDMR | | | |
| | | | FTYVDDKMHDLLGYSPKDLLDTSLFSCQHGADS | | | |
| | | | ERLMATFKSVLSKGQGETSRYRFLGKYGGYCWI | | | |
| | | | LSQATIVYDKLKPQSVVCVNYVISNLENKHEIY | | | |
| | | | SLAQQTAASEQKEQHHQAAETEKEPEKAADPEI | | | |
| | | | IAQETKETVNTPIHTSELQAKPLQLESEKAEKT | | | |
| | | | IEETKTIATIPPVTATSTADQIKQLPESNPYKQ | | | |
| | | | ILQAELLIKRENHSPGPRTITAQLLSGSSSGLR | | | |
| | | | PEEKRPKSVTASVLRPSPAPPLTPPPTAVLCKK | | | |
| | | | TPLQVEPNLPPTTTATAAIISSSNQQLQIAQQT | | | |
| | | | QLQNPQQPAQDMSKGFCSLFADDGRGLTMLKEE | | | |
| | | | PDDLSHHLASTNCIQLDEMTPFSDMLVGLMGTC | | | |
| | | | LLPEDINSLDSTTCSTTASGQHYQSPSSSSTSA | | | |
| | | | PSNTSSSNNSYANSPLSPLTPNSTATASNPSHQ | | | |
| | | | QQQQHHNQQQQQQQQQQHHPQHHDNSNSSSNID | | | |
| | | | PLFNYREESNDTSCSQHLHSPSITSKSPEDSSL | | | |
| | | | PSLCSPNSLTQEDDFSFEAFAMRAPYIPIDDDM | | | |
| | | | PLLTETDLMWCPPEDLQTMVPKEIDAIQQQLQQ | | | |
| | | | LQQQHHQQYAGNTGYQQQQQQPQLQQQHFSNSL | | | |
| | | | CSSPASTVSSLSPSPVQQHHQQQQAAVFTSDSS | | | |
| | | | ELAALLCGSGNGTLSILAGSGVTVAEECNERLQ | | | |
| | | | QHQQQQQQTSGNEFRTFQQLQQELQLQEEQQQR | | | |
| | | | QQQQQQQQQQQQQQQLLSLNIECKKEKYDVQMG | | | |
| | | | GSLCHPMEDAFENDYSKDSANLDCWDLIQMQVV | | | |
| | | | DTEPVSPNAASPTPCKVSAIQLLQQQQQLQQQQ | | | |
| | | | QQQQNIILNAVPLITIQNNKELMQQQQQQQQQQ | | | |
| | | | QQEQLQQPAIKLLNGASIAPVNTKATIRLVESK | | | |
| | | | PPTTTQSRMAKVNLVPQQQQHGNKRHLNSATGA | | | |
| | | | GNPVESKRLKSGTLCLDVQSPQLLQQLIQKDPA | | | |
| | | | QQQTQAAKRAGSERWQLSAESKQQKQQQQQSNS | | | |
| | | | VLKNLLVSGRDDDDSEAMIIDEDNSLVQPIPLG | | | |
| | | | KYGLPLHCHTSTSSVLRDYHNNPLISGTNFQLS | | | |
| | | | PVFGGSDSSGGDGETGSVVSLDDSVPPGLTACD | | | |
| | | | TDASSDSGIDENSLMDGASGSPRKRLSSTSNST | | | |
| | | | NQAESAPPALDVETPVTQKSVEEEFEGGGSGSN | | | |
| | | | APSRKTSISFLDSSNPLLHTPAMMDLVNDDYIM | | | |
| | | | GEGGFEFSDNQLEQVLGWPEIA | | | |
| SWISS_PR | P23497 | Nuclear | MAGGGGDLSTRRLNECISPVANEMNHLPAHSHD | (SEQ ID NO:127) | | |
| OT: | Q13343 | autoantigen Sp-100 | LQRMFTEDQGVDDRLLYDIVFKHFKRNKVEISN | | | |
| SP10_HUM | 075450 | (Speckled | AILKKTFPFLEGLRDRDLITNKMFEDSQDSCRNL | | | |
| AN | Q9UE32 | 100 kDa) | VPVQRVVYNVLSELEKTFNLPVLEALFSDVNMQ | | | |
| | | (Nuclear dot-associated\| | EYPDLIHILYKGFENVIHDKLPLQESEEEEREER | | | |
| | | Sp10 | SGLQLSLEQGTGENSFRSLTWPPSGSPSHAGTT | | | |
| | | 0 protein) | PPENGLSEHPCETEQINAKRKDTTSDKDDSLGS | | | |
| | | (Lysp100h) | QQTNEQCAQKAEPTESCEQIAVQVNNGDAGREM | | | |
| | | | PCPLPCDEESPEAELHNHGIQINSCSVRLVDTK | | | |
| | | | KEKPFSNSKVECQAQARTHHNQASDIIVISSED | | | |
| | | | SEGSTDVDEPLEVFISAPRSEPVINNDNPLESN | | | |
| | | | DEKEGQEATCSRPQIVPEPMDFRKLSTFRESFK | | | |
| | | | KRVIGQDHDFSESSEEEAPAEASSGALRSKHGE | | | |
| | | | KAPMTSRSTSTWRIPSRKRRFSSSDFSDLSNGE | | | |
| | | | ELQETCSSSLRRGSGSQPQEPENKKCSCVNCFP | | | |
| | | | KGVPRSQEARTESSQASDMMDTMDVENNSTLEK | | | |
| | | | HSGKRRKKRRHRSKVNGLQRGRKKDRPRKHLTL | | | |
| | | | NNKVQKKRWQQRGRKANTRPLKRRRKRGPRIPK | | | |
| | | | DENINFKQSELPVTCGEVKGTLYKERFKQGTSK | | | |
| | | | KCIQSEDKKWFTPREFEIEGDRGASKNWKLSIR | | | |
| | | | CGGYTLKVLMENKFLPEPPSTRKKRILESHNNT | | | |
| | | | LVDPCEEHKKKNPDASVKFSEFLKKCSETWKTI | | | |
| | | | FAKEKGKFEDMAKADKAHYEREMKTYIPPKGEK | | | |
| | | | KKKFKDPNAPKRPPLAFFLFCSEYRPKIKGEHP | | | |
| | | | GLSIDDVVKKLAGMWNNTAAADKQFYEKKAAKL | | | |
| | | | KEKYKKDIAAYPAKGKPNSAKKRVVKAEKSKKK | | | |
| | | | KEEEEDEEDEQEEENEEDDDK | | | |
| SWISS_PR | P04267 | Tropomyosin 1, | MEAIKKKMQMLKLDKENAIDRAEQAEANKKQAE | (SEQ ID NO:128) | | |
| OT: | | smooth muscle | DRCKQLEEEQQGLQKKLKGTEDEVEKYSESVKE | | | |
| TPM1_CHI | | (Gizzard beta-tropomyosin) | AQEKLEQAEKKATDAEAEVASLNRRIQLVEEEL | | | |
| CK | | | DRAQERLATALQKLEEAEKAADESERGMKVIEN | | | |
| | | (Smooth-muscle\| | RAMKDEEKMELQEMQLKEAKHIAEEADRKYEEV | | | |
| | | alpha-tropomyosin) | ARKLVVLEGELERSEERAEVAESRVRQLEEELR | | | |
| | TMDQSLKSLIASEEEYSTKEDKYEEEIKLLGEK | | | | | |
| | | (Tropomyosin | LKEAETRAEFAERSVAKLEKTIDDLEESLASAK | | | |
| | | beta chain, | EENVGIHQVLDQTLLELNNL | | | |
| | | smooth muscle) | | | | |
| SWISS_PR | P09493 | Tropomyosin | MDAIKKKMQMLKLDKENALDRAEQAEANKKQAE | (SEQ ID NO:129) | | |
| OT: | | alpha chain, | DRSKQLEDELVSLQKKLKGTEDELDKYSEALKD | | | |
| TPM1_HUM | skeletal muscle | AQEKLELAEKKATDAEADVASLNRRIQLVEEEL | | | | |
| AN | | (Tropomyosin 1, | DRAQERLATALQKLEEAEKAADESERGMKVIES | | | |
| | | skeletal\| | RAQKDEEKMEIQEIQLKEAKHIAEDADRKYEEV | | | |
| | | muscle) | ARKLVIIESDLERAEERAELSEGKCAELEEELK | | | |
| | | | TVTNNLKSLEAQAEKYSQKEDRYEEEIKVLSDK | | | |
| | | | LKEAETRAEPAERSVTKLEKSIDDLEDELYAQK | | | |
| | | | LKYKAISEELDHALNDMTSI | | | |
| SWISS_PR | P04268 | Tropomyosin 2, | MDAIKKKMQMLKLDKENALDRAEQAEDKKAAE | (SEQ ID NO:130) | | |
| OT: | | smooth muscle | ERSKQLEDDIVQLEKQLRVTEDSRDQVLEELHK | | | |
| TPM2 CHI | (Gizzard gamma-tropomyosin) | SEDSLLSAEENAAKAESEVASLNRRIQLVEEEL | | | | |
| CK | | | DRAQERLATALQKLEEAEKAADESERGMKVIEN | | | |
| | | (Smooth-\|muscle | RAQKDEEKMEIQEIQLKEAKHIAEEADRKYEEV | | | |
| | | beta-tropomyosin | ARKLVILEGDLERAEERAELSESKCAELEEELK | | | |
| | | | LVTNEAKSLEAQAEKYSQKEDKYEEEIKVLTDK | | | |
| | | | LKEAETRAEFAERSVTKLEKSIDDLEEKVAHAK | | | |
| | | | EENLNMHQMLDQTLLELNNM | | | |
| SWISS_PR P19353 | Tropomyosin | MAGISSIDAVKKKIQSLQQVADEAEERAEHLQR | (SEQ ID NO:131) | | | |
| OT: | | beta 3, | EADAERQARERAEAEVASLNRRIQLVEEELDRA | | | |
| TPM3_CHI | fibroblast | QERLATALQKLEEAEKAADESERGMKVIENRAM | | | | |
| CK | | | KDEEKMELQEMQLKEAKHIAEEADRKYEEVARK | | | |
| | | | LVVLEGELERSEERAEVAESRVRQLEEELRTMD | | | |
| | | | QSLKSLIASEEEYSTKEDKYEEEIKLLGEKLKE | | | |
| | | | AETRAEFAERSVAKLEKTIDDLEESLASAKEEN | | | |
| | | | VGIHQVLDQTLLELNNL | | | |
| SWISS_PR | | | P06753 | Tropomyosin | MEAIKKKMQMLKLDKENALDRAEQAEAEQKQAE | (SEQ ID NO:132) |
| OT: | | alpha chain, | ERSKQLEDELAANQKKLKGTEDELDKYSEALKD | | | |
| TPM3_HUM | | skeletal muscle | AQEKLELAEKKAADAEAEVASLNRRIQLVEEEL | | | |
| AN | | type | DRAQERLATALQKLEEAEKAADESERGMKVIEN | | | |
| | | (Tropomyosin | 3, RALKDEEKMELQEIQLKEAKHIAEEADRKYEEV | | | |
| | | skeletal\| | ARKLVIIEGDLERTEERAELAESKCSELEEELK | | | |
| | | muscle) | NVTNNLKSLEAQAEKYSQKEDKYEEEIKILTDK | | | |
| | | | LKEAETRAEFAERSVAKLEKTIDDLEDELYAQK | | | |
| | | | LKYKAISEELDHALNDMTSI | | | |
| SWISS_PR | P49438 | Tropomyosin | MAALSSLEAVRKKIRSLQEQAAEERAGKLQR | (SEQ ID NO:133) | | |
| OT: | | alpha chain, | EVDQERALREEAESEVASLNRRIQLVEEELDRA | | | |
| TPM5_CHI | | major brain | QERLATALQKLEEAEKAADESERGMKVIENPAQ | | | |
| CK | | isoform | KDEEKMEIQEIQLKEAKHIAEEADRKYEEVANK | | | |
| | | | LVIIEGDLERAEERAELSESKCAELEEELKTVT | | | |
| | | | NNLKSLEAQAEKYSQKEDKYEEEIKVLTDKLKE | | | |
| | | | AETRAEFAERSVTKLEKSIDDLEDQLYQQLEQN | | | |
| | | | SRLTNELKLALNED | | | |
| SWISS_PR | P49439 | Tropomyosin | MDATKKKMQMLKLDKENALDRAEQAEADKKAAE | (SEQ ID NO:134) | | |
| OT: | | alpha chain, | ERSKQLEDELVALQKKLKCTEDELDKYSESLKD | | | |
| TPM6_CHI | minor brain | AQEKLELADKKATDAESEVASLNRRIQLVEEEL | | | | |
| CK | | isoform | DRAQERLATALQKLEEAEKAADESERGMKVTEN | | | |
| | | | RAQKDEEKMEIQEIQLKEAKHIAEEADRKYEEV | | | |
| | | | ARKLVIIECDLERAEERAELSESKCAELEEELK | | | |
| | | | TVTNNLKSLEAQAEKYSQKEDKYEEEIKVLTDK | | | |
| | | | LKEAETRAEFAERSVTKLEKSIDDLEDQLYQQL | | | |
| | | | EQNSRLTNELKLALNED | | | |
| SWISS_PR | P13104 | Tropomyosin | MDAIKKKMQMLKLDKENALDRAEQAETDKKAAE | (SEQ ID NO:135) | | |
| OT: | | alpha chain, | ERSKQLEDDLVALQKKLKATEDELDKYSEALKD | | | |
| TPMA_BRA | skeletal muscle | AQEKLELAEKKATDAEGDVASLNRRIQLVEEEL | | | | |
| RE | | | DRAQERLATALQKLEEAEKAADESERGMKVIEN | | | |
| | | | RALKDEEKMELQEIQLKEAKHIAEEADRKYEEV | | | |
| | | | ARKLVIVEGELERTEERAELNEGKCSELEEELK | | | |
| | | | TVTNNMKSLEAQAEKYSAKEDKYEEEIKVLTDK | | | |
| | | | LKEAETRAEFAERSVAKLEKTIDDLEDELYAQK | | | |
| | | | LKYKAISEELDHALNDMTSI | | | |
| SWISS_PR | P02559 | Tropomyosin | MDAIKKKMQMLKLDKENALDRAEQAEADKKAAE | (SEQ ID NO:136) | | |
| OT: | P18442 | alpha chain, | ERSKQLEDELVALQKKLKCTEDELDKYSESLKD | | | |
| TPMA_COT | | skeletal muscle | AQEKLELADKKATDAESEVASLNRRIQLVEEEL | | | |
| JA | | | DRAQERLATALQKLEEAEKAADESERGMKVTEN | | | |
| | | | RAQKDEEKMEIQEIQLKEAKHIAEEADRKYEEV | | | |
| | | | ARKLVIIEGDLERAEERAELSESKCAELEEELK | | | |
| | | | TVTNNLKSLEAQAEKYSQKEDKYEEEIKVLTDK | | | |
| | | | LKEAETRAEFAERSVTKLEKSIDDLEDELYAQK | | | |
| | | | LKYKAISEELDHALNDMTSI | | | |
| SWISS_PR | P02558 | Tropomyosin | MDAIKKKMQMLKLDKENALDRAEQAEADKKAAE | (SEQ ID NO:137) | | |
| OT: | P46902 | alpha chain, | DRSKQLEDELVSLQKKLKGTEDELDKYSEALKD | | | |
| TPMA_MOU | P99034 | skeletal and | AQEKLELAEKKATDAEADVASLNRRIQLVEEEL | | | |
| SE | | cardiac muscle | DRAQERLATALQKLEEAEKAAIDESERQMKVIES | | | |
| | | | RAQKDEEKMEIQEIQLKEAKHIAEDADRKYEEV | | | |
| | | | ARKLVIIESDLERAEERAELSEGKCAELEEELK | | | |
| | | | TVTNNLKSLEAQAEKYSQKEDKYEEEIKVLSDK | | | |
| | | | LKEAETRAEFAERSVTKLEKSIDDLEDELYAQK | | | |
| | | | LKYKAISEELDHALNDMTSI | | | |
| SWISS_PR | P13105 | Tropomyosin | MDAIKKKMQMLKLDKENALDRAEQAEADKKGAE | (SEQ ID NO:138) | | |
| OT: | | alpha chain, | DKSKQLEDELVAMQKKMKGTEDELDKYSEALKD | | | |
| TPMA_RAN | skeletal muscle | AQEKLELAEKKATDAEADVASLNRRIQLVEEEL | | | | |
| TE | | | DRAQERLATALQKLEEAEKAADESERGMKVIEN | | | |
| | | | RALKDEEKIELQEIQLKEAKHIAEEADRKYEEV | | | |
| | | | ARKLVIIEGDLERAEERAELSESKCAELEEELK | | | |
| | | | TVTNNLKSLEAQAEKYSQKEDKYEEEIKVLTDK | | | |
| | | | LKEAETRAEFAERTVAKLEKSIDDLEDELYAQK | | | |
| | | | LKYKAISEELDHALNDMTSI | | | |
| SWISS_PR | P04692 | Tropomyosin | MDAIKKKMQMLKLDKENALDRAEQAEADKKAAE | (SEQ ID NO:139) | | |
| OT: | | alpha chain, | DRSKQLEDELVSLQKKLKGTEDELDKYSEALKD | | | |
| TPMA_RAT | | skeletal muscle | AQEKLELAEKKATDAEADVASLNRRIQLVEEEL | | | |
| | | | DRAQERLATALQKLEEAEKAADESERGMKVIES | | | |
| | | | RAQKDEEKMEIQEIQLKEAKHIAEDADRKYEEV | | | |
| | | | ARKLVIIESDLERAEERAELSEGKCAELEEELK | | | |
| | | | TVTNNLKSLEAQAEKYSQKEDKYEEEIKVLSDK | | | |
| | | | LKEAETRAEFAERSVTKLEKSIDDLEDELYAQK | | | |
| | | | LKYKAISEELDHALKDMTSI | | | |
| SWISS_PR Q01173 | Tropomyosin | MDAIKKKMQMLKLDKENALDRAEQAEADKKGAE | (SEQ ID NO:140) | | | |
| OT: | | alpha chain, | DKSKQLEDELVALQKKLKGTEDELDKYSEALKD | | | |
| TPMA_XEN | | skeletal muscle | AQEKLELSDKKATDAEGDVASLNRRIQLVEEEL | | | |
| LA | | DRAQERLSTALQKLEEAEKAADESERGMKVIEN | | | | |
| | | | RALKDEEKMELQEIQLKEAKHIAEEADRKYEEV | | | |
| | | | ARKLVIIEGDLERAEERAELSESKCAELEEELK | | | |
| | | | TVTNNLKSLEAQAEKYSQKEDKYEEEIKVLTDK | | | |
| | | | LKEAETRAEFAERTVAKLEKSIDDLEDELYAQK | | | |
| | | | LKYKAISEELDHALNDMTSI | | | |
| SWISS_PR | P19352 | Tropomyosin | MEAIKKKMQMLKLDKENAIDRAEQAEADKKQAE | (SEQ ID NO:141) | | |
| OT: | | beta chain, | DRCKQLEEEQQGLQKKLKGTEDEVEKYSESVKE | | | |
| TPME_CHI | | skeletal muscle | AQEKLEQAEKKATDAEAEVASLNRRIQLVEEEL | | | |
| CK | | | DRAQERLATALQKLEEAEKAADESERGMKVIEN | | | |
| | | | RAMKDEEKMELQEMQLKEAKHIAEEADRKYEEV | | | |
| | | | ARKLVVLECELERSEERAEVAESKCGDLEEELK | | | |
| | | | IVTNNLKSLEAQADKYSTKEDKYEEEIKLLGEK | | | |
| | | | LKEAETRAEFAERSVAKLEKTIDDLEDEVYAQK | | | |
| | | | MKYKAISEELDNALNDITSL | | | |
| SWISS_PR | P07951 | Tropomyosin | MDAIKKKMQMLKLDKENAIDRAEQAEADKKQAE | (SEQ ID NO:142) | | |
| OT: | | beta chain, | DRCKQLEEEQQALQKKLKGTEDEVEKYSESVKE | | | |
| TPMB_HUM | | skeletal muscle | AQEKLEQAEKKATDAEADVASLNRRIQLVEEEL | | | |
| AN | | | (Tropomyosin 2, | DRAQERLATALQKLEEAEKAADESERGMKVIEN | | |
| | | skeletal | RAMKDEEKMELQEMQLKEAKHIAEDSDRKYEEV | | | |
| | | muscle) | ARKLVILEQELERSEERAEVAESKCGDLEEELK | | | |
| | | | IVTNNLKSLEAQADKYSTKEDKYEEEIKLLEEK | | | |
| | | | LKEAETRAEFAERSVAKLEKTIDDLEDEVYAQK | | | |
| | | | MKYKAISEELDNALNDITSL | | | |
| SWISS_PR | P02560 | Tropomyosin | MDAIKKKNQMLKLDKENAIDRAEQAEADKKQAE | (SEQ ID NO:143) | | |
| OT: | | beta chain, | DRCKQLEEEQQALQKKLKQTEDEVEKYSESVKD | | | |
| TPMB_MOU | | skeletal muscle | AQEKLEQAEKKATDAEADVASLNRRIQLVEEEL | | | |
| SE | | | DRAQERLATALQKLEEAEKAADESERGMKVIEN | | | |
| | | | RAMKDEEKMELQEMQLKEAKHIAEDSDRKYEEV | | | |
| | | | ARKLVILEGELERSEERAEVAESKCQDLEEELK | | | |
| | | | IVTNNLKSLEAQADKYSTKEDKYEEEIKLLEEK | | | |
| | | | LKEAETRAEFAERSVAKLEKTIDDLEDEVYAQK | | | |
| | | | MKYKAISEELDNALNDITSL | | | |
| SWISS_PR P42639 | Tropomyosin | MDAIKKKMQMLKLDKENALDRADDEADKKAAE | (SEQ ID NO:144) | | | |
| OT: | | alpha chain, | DRSKQLEDELVSLQKKLKATEDELDKSEALKD | | | |
| TPMC_PIG | | cardiac muscle | AQEKLELAEKKATDAEADVASLNRRIQLFEEEL | | | |
| | | | DRAQERLATALQKLEEAEKAADESERGMKVIES | | | |
| | | | RAQKDEEKMEIQEIQLKEAKHIAEDADRKYEEV | | | |
| | | | ARKLVIIESDLERAEERAELSEGKCAELEEELK | | | |
| | | | TVTNNLKSLEAQAEKYSQKEDKYEEEIKVLSDK | | | |
| | | | LKEAETRAEFAERSVTKLEKSIDDLEDELYAQK | | | |
| | | | LKYKAISEELDHALNDMTSI | | | |
| SWISS_PR | P18441 | Tropomyosin | MDAIKKKMQMLKLDKENALDPAEQAEADKKAAE | (SEQ ID NO:145) | | |
| OT: | | alpha chain, | ERSKQLEDELVALQKKLKGTEDELDKYSESLKD | | | |
| TPMF_CHI | | fibroblast | AQEKLELADKKATDAESEVASLNRRIQLVEEEL | | | |
| CK | | isoform F1 | DRAQERLATALQKLEEAEKAADESERGMKVIEN | | | |
| | | | RAQKDEEKMEIQEIQLKEAKHIAEEADRKYEEV | | | |
| | | | ARKLVIIEGDLERAEERAELSESQVRQLEEQLR | | | |
| | | | IMDQTLKALMAAEDKYSQKEDKYEEEIKVLTDK | | | |
| | | | LKEAETRAEFAERSVTKLEKSIDDLEEKVAHAK | | | |
| | | | EENLNMHQMLDQTLLELNNM | | | |
| SWISS_PR | P08942 | Tropomyosin | MDAIKKKMQMLKLDKENALDPAEQAEADKKAAE | (SEQ ID NO:146) | | |
| OT: | | alpha chain, | ERSKQLEDELVALQKKLKGTEDELDKYSESLKD | | | |
| TPMG_COT | | fibroblast | AQEKLELADKKATDAESEVASLNRRIQLVEEEL | | | |
| JA | | isoform F2 | DRAQERLATALQKLEEAEKAALESERGMKVIEN | | | |
| | | | RAQKDEEKNEIQEIQLKEAKHIAEEADRKYEEV | | | |
| | | | ARKLVIIECDLERAEERAELSESKCAELEEELK | | | |
| | | | TVTNNLKSLEAQAEKYSQKEDKYEEEIKVLTDK | | | |
| | | | LKEAETRAEFAERSVTKLEKSIDDLEEKVAHAK | | | |
| | | | EENLNMHQMLDQTLLELNNM | | | |
| SWISS_PR | Q01174 | Tropomyosin | MAGITSLEAVKRKIKCLQDQADEAEERAEKLQR | (SEQ ID NO:147) | | |
| OT: | | alpha chain, | ERDMERKLREAAEQDVASLNRRIQLVEEELDPRA | | | |
| TPMN_XEN | | non-muscle | QERLSTALQKLEEAEKAADESERGMKVIENRAL | | | |
| LA | | | KDEEKMELQEIQLKEAKHIAEEADRKYEEVARK | | | |
| | | | LVIIEQDLEPAEERAELSESHYRQLEDQQRIMD | | | |
| | | | QTLKTLIASEEKYSQKEDKYEEEIKVLTDKLKE | | | |
| | | | AETRAEFAERTVAKLEKSIDDLEEKVAHAKEEN | | | |
| | | | LNMHQIVILDQTLLELNNM | | | |
| SWISS_PR | P49436 | Tropomyosin | MDAIKKKMQMLKLDKENALDRAEQAEADKKAAE | (SEQ ID NO:148) | | |
| OT: | | alpha chain, | ERSKQLEDDIVQLEKQLRVTEDSRDQVLEELHK | | | |
| TPMS_CHI | | smooth muscle | SEDSLLFAEENAAKAESEVASLNRRIQLVEEEL | | | |
| CK | | | DRAQERLATALQKLEEAEKAADESERGMKVIEN | | | |
| | | | RAQKDEEKMEIQEIQLKEAKHIAEEADRKYEEV | | | |
| | | | ARKLVIIEGDLERAEERAELSESKCAELEEELK | | | |
| | | | TVTNNLKSLEAQAEKYSQKEDKYEEEIKVLTDK | | | |
| | | | LKEAETRAEFAERSVTKLEKSIDDLEEKVARAK | | | |
| | | | EENLNMNQMLDQTLLELNNM | | | |
| SWISS_PR | P49437 | Tropomyosin | MDAIKKKMQMLKLDKENALDPAEQAEPLKKAAE | (SEQ ID NO:149) | | |
| OT: | | alpha chain, | ERSKQLEDDIVQLEKQLRVTEDSRDQVLEELHK | | | |
| TPMS_COT | | smooth muscle | SEDSLLSAEEIAAKAESEVASLNRRIQLVEEEL | | | |
| JA | | | DRAQERLATALQKLEEAEKAADESERCMKVIEN | | | |
| | | | RAQKDEEKMEIQEIQLKEAKHIAEEIWRKYEEV | | | |
| | | | ARKLVIIEGDLERAEERAELSESKCAELEEELK | | | |
| | | | TVTNNLKSLEAQAEKYSQKEDKYEEEIKVLTDK | | | |
| | | | LKEAETRAEFAERSVTKLEKSIDDLEEKVAHAK | | | |
| | | | EENLNMHQMLDQTLLELNNM | | | |
| SWISS_PR | P10469 | Tropomyosin | CRLRIFLRTASSEHLHERKLRETAEDVASLNR | (SEQ ID NO:150) | | |
| OT: | | alpha chain, | RIQLVEEELDRAQERLATVLQKLEEAEKAALES | | | |
| TPMS_HUM | | smooth muscle | ERGMKVTESRAQKDEEKMEIQEIQLKEAKHIAE | | | |
| AN | | (Tropomyosin 1, | DADRKYEEVARKLVITESDIIEPAEERAELSEGQ | | | |
| | | smooth muscle) | VRQLEEQLRIMDSDLESINAAEDKYSQKEDRYE | | | |
| | | (Fragment) | EEIKVLSDKLKEAETRAEFAERSVTKLEKSIDD | | | |
| | | | LEEKVAHAKEENLSMHQMLDQTLLELNNM | | | |
| SWISS_PR | P06469 | Tropomyosin | MDAIKKKMQMLKLDKENALDRAEQAEADKKAAE | (SEQ ID NO:151) | | |
| OT: | | alpha chain, | DRSKQLEEDISAKEKLLRASEDERDRVLEELHK | | | |
| TPMS_RAT | | smooth muscle | AEDSLLAADETAAKAEADVASLNRRIQLVEEEL | | | |
| | | | DRAQERLATALQKLEEAEKAADESERGMKVIES | | | |
| | | | RAQKDEEKMEIQEIQLKEAKHIAEDADRKYEEV | | | |
| | | | ARKLVIIESDLERAEERAELSEGKCAELEEELK | | | |
| | | | TVTNNLKSLEAQAEKYSQKEDKYEEEIKVLSDK | | | |
| | | | LKEAETRAEFAERSVTKLEKSIDDLEEKVAHAK | | | |
| | | | EENLSMHQMLHQTLLELNNM | | | |
| SWISS_PR | P18342 | Tropomyosin | MDAIKKKMQMLKLDKENALDPAEQAEADKKAAE | (SEQ ID NO:152) | | |
| OT: | | alpha chain, | DRSKQLEDELVSLQKKLKATEDELDKYSEALKL | | | |
| TPMX_RAT | | brain-1 | AQEKLELAEKKATDAEADVASLNRRIQLVEEEL | | | |
| | | (TMBR-1) | DRAQERLATALQKLEEAEKAADESERGMKVIES | | | |
| | | | RAQKDEEKMEIQEIQLKEAKHIAEDADRKYEEV | | | |
| | | | ARKLVIIESDLERAEERAELSECKCAELEEELK | | | |
| | | | TVTNNLKSLEAQAEKYSQKEDKYEEEIKVLSDK | | | |
| | | | LKEAETRAEFAERSVTKLEKSIDDLEDQLYHQL | | | |
| | | | EQNRRLTNELKLALNED | | | |
| SWISS_PR | P18343 | Tropomyosin | MAGSSSLEAVRRKIRSLQEQADAAEERAGSLQR | (SEQ ID NO:153) | | |
| OT: | | alpha chain, | ELDQERKLRETAEADVASLNRRIQLVEEELDRA | | | |
| TPMY_RAT | | brain-2 | QERLATALQKLEEAEKAADESERGMKVIESRAQ | | | |
| | | (TMBR-2) | KDEEKMEIQEIQLKEAKHIAEDADRKYEEVANK | | | |
| | | | LVIIESDLERAEERAELSEQKCAELEEELKTVT | | | |
| | | | NNLKSLEAQAEKYSQKEDKYEEEIKVLSDKLKE | | | |
| | | | AETRAEFAERSVTKLEKSIDDLEDKFLCFSPPK | | | |
| | | | TPSSSRMSHLSELCICLLSS | | | |
| SWISS_PR | P18344 | Tropomyosin | MAGSSSLEAVRRKIRSLQEQADAAEERAGSLQR | (SEQ ID NO:154) | | |
| OT: | | alpha chain, | ELDQERKLRETAEADVASLNRRIQLVEEELDRA | | | |
| TPMZ_RAT | | brain-3 (TMBR-3) | QERLATALQKLEEAEKAADESERGMKVIESRAQ | | | |
| | | | KDEEKMEIQEIQLKEAKHIAEDADRKYEEVANK | | | |
| | | | KDEEKMEIQEIQLKEAKHIAEDADRKYEEVARK | | | |
| | | | LVIIESDLERAEERAELSEGKCAELEEELKTVT | | | |
| | | | NNLKSLEAQAEKYSQKEDKYEEEILKVLSDKLKE | | | |
| | | | AETRAEFAERSVTKLEKSIDDLEDQLYHQLEQN | | | |
| | | | RRLTNELKLALNED | | | |
| SWISS_PR | P41541 | General | MNFLRGVMGGQSAGPQHTEAETIQKLCDRVASS | (SEQ ID NO:155) | | |
| OT: | | vesicular | TLLDDRRNAVRALKSLSKKYRLEVGIQANEHLI | | | |
| VDP_BOVI | | transport | HVLQTDRSDSEIIGYALDTLYNIISNDEEEEVE | | | |
| N | | factor p115 | ENSTRQSEDLGSQFTEIFIKQQENVTLLLSLLE | | | |
| | | (Transcytosis | EFDFHVRWPGVKLLTSLLKQLGPQVQQIILVSP | | | |
| | | associated | MGVSRLMDLLADSREVIRNDGVLLLQALTRSNG | | | |
| | | protein) (TAP) | AIQKIVAFENAFERLLDIITEEGNSDGGIVVED | | | |
| | | (Vesicle | CLILLQNLLKNNNSNQNFFKEGSYIQRMKPWFE | | | |
| | | docking | VGDENSGWSAQKVTNLHLMLQLVRVLVSPNNPP | | | |
| | | protein) | GATSSCQKAMFQCGLLQQLCTILMATGVPADIL | | | |
| | | | TETINTVSEVIRGCQVNQDYFASVNAPSNPPRP | | | |
| | | | AIVVLLMSMVNERQPFVLRCAVLYCFQCFLYKN | | | |
| | | | QKGQGEIVSTLLPSTIDATGNTVSAGQLLCGGL | | | |
| | | | FSTDSLSNWCAAVALAHALQENATQKEQLLRVQ | | | |
| | | | LATSIGNPPVSLLQQCTNILSQGSKIQTRVGLL | | | |
| | | | MLLCTWLSNCPIAVTHFLHNSANVPFLTGQIAE | | | |
| | | | NLGEEEQLVQGLCALLLGISIYFNDNSLETYMK | | | |
| | | | EKLKQLIEKRIGKENFIEKLGFISKHELYSRAS | | | |
| | | | QKPQPNFPSPEYMIFDHEFTKLVKELEGVITKA | | | |
| | | | IYKSSEEDKKEEEVKKTLEQHDSIVTHYKNMIR | | | |
| | | | EQDLQLEELKQQISTLKCQNEQLQTAVTQQVSQ | | | |
| | | | IQQHKDQYNLLKVQLGKDSQHQGPYTDGAQMNG | | | |
| | | | VQPEEISRLREEIEELKSNRELLQSQLAEKDSL | | | |
| | | | IENLKSSQLSPGTNEQSSATAGDSEQIAELKQE | | | |
| | | | LATLKSQLNSQSVEITKLQTEKQELLQKTEAFA | | | |
| | | | KSAPVPGESETVIATKTTDVEGRLSALLQETKE | | | |
| | | | LKNEIKALSEERTAIKEQLDSSNSTIAILQNEK | | | |
| | | | NKLEVDITDSKKEQDDLLVLLADQDQKIFSLKN | | | |
| | | | KLKELGHPVEEEDELESGDQDDEDDEDEDDGKE | | | |
| | | | QGHI | | | |
| SWISS_PR | P41542 | General | MNFLRGVMGGQSAGPQHTEAETIQKLCDRVASS | (SEQ ID NO:156) | | |
| OT: | | vesicular | TLLDDRRNAVRALKSLSKKYRLEVGIQAMEHLI | | | |
| VDP_RAT | | transportHVLQTDRSDSEIIAYALDTLYNIISNDEEEEVE | | | | |
| | | factor p115 | ENSTRQSEDLGSQFTEIFIKQPENVTLLLSLLE | | | |
| | | (Transcytosis | EFDFHVRWPGVRLLTSLLKQLGPPVQQIILVSP | | | |
| | | associated\|prot | MGVSKLMDLLADSREIIRNDGVLLLQALTRSNG | | | |
| | | ein) (TAP) | AIQKIVAFENAFERLLDIITEEGNSDGGIVVED | | | |
| | | (Vesicle | CLILLQNLLKNNNSNQNFFKEGSYIQRMKAWFE | | | |
| | | docking | VGDENPGWSAQKVTNLHLMLQLVRVLVSPTNPP | | | |
| | | protein) | GATSSCQKAMFQCGLLQQLCTILMATGIPADIL | | | |
| | | | TETINTVSEVIRGCQVNQDYFASVNAPSNPPRP | | | |
| | | | AIVVLLMSMVNERQPFVLRCAVLYCFQCFLYKN | | | |
| | | | EKGQGEIVATLLPSTIDATGNSVSAGQLLCGGL | | | |
| | | | FSTDSLSNWCAAVALAHALQGNATQKEQLLRVQ | | | |
| | | | LATSIGNPPVSLLQQCTNILSQGSKIQTRVGLL | | | |
| | | | MLLCTWLSNCPIAVTHFLHNSANVPFLTGQIAE | | | |
| | | | NLGEEEQLVQGLCALLLGISIYFNDNSLENYTK | | | |
| | | | EKLKQLIEKRIGKENYIEKLGFISKHELYSRAS | | | |
| | | | QKPQPNFPSPEYMIFDHEFTKLVKELEGVITKA | | | |
| | | | IYKSSEEDKKEEEVKKTLEQHDNIVTHYKNMIR | | | |
| | | | EQDLQLEELKQQVSTLKCQNEQLQTAVTQQASQ | | | |
| | | | TQQHKDQYNLLKVQLGKLNHHQGSHSDGAQASG | | | |
| | | | IQPEEISRLREEIEELRSHQVLLQSQLAEKDTV | | | |
| | | | IENLRSSQVSGMSEQALATCSPRDAEQVAELKQ | | | |
| | | | ELSALKSQLCSQSLEITRLQTENSELQQRAETL | | | |
| | | | AKSVPVEGESELVTAAKTTDVEGRLSALLQETK | | | |
| | | | ELKNEIKALSEERTAIQKQLDSSNSTIAILQTE | | | |
| | | | KDKLYLEVTDSKKEQDDLLVLLADQDQKILSLK | | | |
| | | | SKLKDLGHPVEEEDESGDQEDDDDELDDGDRDQ | | | |
| | | | DI | | | |
| TREMBL: | Q21049 | PUTATIVE LIPRIN | MSYSNGNINCDIMPTISEDGVDNGGPIDEPSDR | (SEQ ID NO:157) | | |
| Q21049 | | ALPHA (LAR-INTERACTING | DNIEQLMMNMLEDRDKLQEQLENYKVQLENAGL | | | |
| | | | RTKEVEKERDMMKRQFEVHTQNLPQELQTMTRE | | | |
| | | PROTEIN ALPHA) | LCLLKEQLLEKDEEIVELKAERNNTRLLLEHLE | | | |
| | | | CLVSRHERSLRMTVMKRQAQNHAGVSSEVEVLK | | | |
| | | | ALKSLFEHHKALDEKVRERLRVAMERVATLEEE | | | |
| | | | LSTKGDENSSLKARIATYAAEAEEAMASNAPIN | | | |
| | | | GSISSESANRLIEMQEALERMKTELANSLKQST | | | |
| | | | EITTRNAELEDQLTEDAREKHAAQESIVRLKNQ | | | |
| | | | ICELDAQRTDQETRITTFESRFLTAQRESTCIR | | | |
| | | | DLNDKLEHQLANKDAAVRLNEEKVHSLQERLEL | | | |
| | | | AEKQLAQSLKKAESLPSVEAELQQRMEALTAAE | | | |
| | | | QKSVSAEERIQRLDNIQELSAELERAVQRERM | | | |
| | | | NEEHSQRLSSTVDKLLSESNDRLQLHLKERMQA | | | |
| | | | LDDKNRLTQQLDGTKKYDQAERIKDRLQRDNE | | | |
| | | | SLRQEIEALRQQLYNARTAQFQSRMHAIPFTHA | | | |
| | | | QNIVQQQPQASIAQQSAYQMYKQQPAQQYQTVG | | | |
| | | | MRRPNKGRISALQDDPNKVQTLNEQEWDRLQQA | | | |
| | | | HVLANVQQAFSSSPSLADVGQSTLPRPNTAVQH | | | |
| | | | QQDDMMNSGMGMPSGMQGGMQGGMGGGQDAQML | | | |
| | | | ASMLQDRLDAINTEIRLIQQEKHHAERVAEQLE | | | |
| | | | RSSREFYDDQGISTRSSPRASPQLDNMRQHKYN | | | |
| | | | TLPANVSGDRRYDIYGNPQFVDDRMVRDLDYEP | | | |
| | | | RRGYNQFDEMQYERDRMSPASSVASSTDGVLGG | | | |
| | | | KKKRSNSSSGLKTLGRFFNKKKNSSSDLFKRNG | | | |
| | | | DYSDGEQSGTEGNQKADYDRRKKKKHELLEEAM | | | |
| | | | KARTPFALWNGPTVVAWLELWVGMPAWYVAACR | | | |
| | | | ANVKSGAIMSALSDQEIQKEIGISNPLHRLKLR | | | |
| | | | LAIQEMVSLTSPSAPRTARLTLAFGDMNHEYIG | | | |
| | | | NDWLPCLGLAQYRSAFMECLLDARMLEHLSKED | | | |
| | | | LRTHLRMVDTFHRTSLQYGIMCLKKVNYDKKVL | | | |
| | | | ADRRKACDNINTDLLVWSNERVQRWVEEIGLGV | | | |
| | | | FSRNLVDSGIHGALIALDETFDASAFAYALQIG | | | |
| | | | SQDVPNRQLLEKKFIGLVNDHRQQSDPHPRSGS | | | |
| | | | SRKNDSIAKSYEFHLYT | | | |
| TREMBL: | Q94071 | PUTATIVE LIPRIN | MYSRHSISDAYGAVCILPEDTLTVSSSQNSHID | (SEQ ID NO:158) | | |
| Q94071 | | BETA (LAR-INTERACTING | AFAALVDRERDSSRSSGSGNIFKDNGSIKRRQA | | | |
| | | | LPYVTHYSDSGFGSAPSAGSSCSYLPPPPPYRM | | | |
| | | PROTEIN BETA) | RGSGCLSSKPQHKIHRSLEDSKYTASLMTTGVP | | | |
| | | | TLPLLSMTPFNQLQSRDARGASWISLVRAPNFH | | | |
| | | | LYCFFVFFFSFNIDETFRNSNISSPSPSMSTVS | | | |
| | | | CPEYPELQDKLHRLAMARDSLQLQVSVLSEQVG | | | |
| | | | AQKEKIKDLETVIALKRNNLTSTEELLQDKYHR | | | |
| | | | IDECQELESKKMDLLAEVSSLKLRYATLEREKN | | | |
| | | | ETEKKLRLSQNEMDHVNQSMHGMVVQQQLAHHT | | | |
| | | | NGHESGGYMEPLREHRSEKNDDEMSQLRTAVQR | | | |
| | | | LMADNEHKELQINTLRNALDEQMRSRSQQEDFY | | | |
| | | | ASQRNYTDNFDVNAQIRRILMDEPSDSMEHSTS | | | |
| | | | FPVSLSSTTSNGKGPRSTVQSSESYNSSLSAVS | | | |
| | | | PQHNWSSAGAGTPRQLHPIGGNQRVNNITSAQY | | | |
| | | | CSPSPPAARQLAAELDELRRNGNEGANHNYSSA | | | |
| | | | SLPRGVGKASSTLTLPSKKLSVASGTSVVESDD | | | |
| | | | EIARGRNLNNATSQSNLKNFSRERTRSSLRNIF | | | |
| | | | EKLTRSTSQDQSNSFRRGSAARSTSTARLGSTN | | | |
| | | | HLGTVSKRPPLSQFVDWRSEQLADWIAEIGYPQ | | | |
| | | | YMNEVSRHVRSGRHFLNMSMNEYEGVLNIKNPV | | | |
| | | | HRKRVAILLRRIEEDIMEPANKWDVHQTLRWLD | | | |
| | | | DIGLPQYKDVFAENVVDGPLLLSLTANDAVEMK | | | |
| | | | VVNAHHYATLARSIQFLKKADFRFNAMEKLIDQ | | | |
| | | | NIVEKYPCPDVVVRWTHSATCEWLRKIDLAEFT | | | |
| | | | QNLLFAGVPGALMIYEPSFTAESLAEILQMPPH | | | |
| | | | KTLLRRHLTSHFNQLLGPKIIADKRDFLAAGNY | | | |
| | | | PQISPTGRVKVVKKGFSLTRKKAKNEICLEPEE | | | |
| | | | LLCPQVLVHKYPTGAGDNSSFEESNV | | | |

High stability leucine zippers may be derived using procedures known to those of ordinary skill in the art (see, e.g., Newman et al., 2000, A computationally directed screen identifying interacting coiled coils from Saccharomyces cerevisiae, Proc. Natl. Acad. Sci. USA 97, 13203-08). Computer programs such as PAIRCOIL (Berger et al., 1995, Predicting Coiled Coils by Use of Pairwise Residue Correlations, Proc. Natl. Acad. Sci. USA, 92: 8259-63) and MULTICOIL (Wolf et al., 1997, MultiCoil: A program for predicting two- and three-stranded coiled coils, Protein Science 6: 1179-89), may be used to predict how coiled coils will interact to form dimers and/or trimers, etc.

Leucine zippers can be described as seven residue repeat units. Of the seven amino acids in each heptad derived from a leucine zipper, the residues in the a and d positions are generally hydrophobic amino acids (alanine, valine, phenylalanine, methionine, isoleucine and leucine) while the amino acids in the e and g positions are usually charged amino acids (aspartic acid, glutamic acid, lysine and arginine). The specific sequence of hydrophobic a and d residues determines whether two members of a pair interact. Accordingly, many coiled coils are already known and computer software analyses may be used to identify, design, and test potential novel coiled coils (Newman et al., 2000, A computationally directed screen identifying interacting coiled coils from Saccharomyces cerevisiae, Proc. Natl. Acad. Sci. USA 97: 13203-08).

The residues in the e and g position of the heptad determine how strongly coiled coils bind to each other by forming salt bridges that stabilize the binding between the coils (see, e.g., Krylov et al., 1998, Interhelical interactions in the leucine zipper coiled coil dimer: pH and salt dependence of coupling energy between charged amino acids, J. Mol. Biol. 279: 959-72). Predictive formulae for interhelical binding strength of leucine zippers based on the zipper sequence, particularly the e and g positions, have been derived, and are known to those of skill in the art. These can be used to determine the length of the leucine zipper needed for construction of a particular assembly unit. The number of heptads in a leucine zipper affects the binding strength between molecules comprising those heptads; generally, about four heptads are sufficient at normal temperatures. In certain embodiments, nanostructures that will be subjected to higher temperatures (>40° C.) are constructed using assembly units comprising longer coiled coils or coiled coils stabilized in another manner such as, but not limited to, the introduction of one or more intermolecular disulfide bonds.

Isolated leucine zippers generally do not form stable dimers outside of a protein milieu (Branden and Tooze 1999, Introduction to Protein Structure, 2nd ed., Garland Publishing, Inc. New York, p. 37). Therefore, in order to stabilize assembly units of the invention that are formed with leucine zippers, flanking cysteines are inserted, in preferred embodiments, to form disulfide bridges. Once these bonds have formed, the designed assembly units should be stable unless exposed to reducing agents. Therefore, in certain embodiments, cysteines are added to the end of the leucine zipper or between the α-helix of a leucine zipper and a PNA joining element, for the formation of stabilizing disulfide bonds.

The precise position of the cysteines in an assembly unit can be determined by modeling the assembly unit or assembly subunits using molecular modeling software such as SIBYL (Tripos Inc., St. Louis, Mo.), RasMol (Sayle et al., 1995, RasMol: Biomolecular graphics for all, Trends Biochem. Sci. (TIBS) 20(9): 374-76), or PdbMotif (Saqi et al., 1994, PdbMotif—a tool for the automatic identification and display of motifs in protein structures, Comput. Appl. Biosci. 10(5): 545-46), and then tested empirically. Conversion of two cysteines into a disulfide bridge is well-known to those skilled in the art and is controlled by altering the redox potential of the solution. Under oxidizing conditions (e.g. in the presence of oxygen) the sulfur atoms will bond. Under reducing conditions (e.g. with the addition of a reducing agent such as dithiothreitol (DTT)) the two sulfur atoms will not bond together.

Generally, two disulfide bonds are sufficient to hold the coiled-coils of an assembly unit together. In preferred embodiments, the cysteine residues are disposed at the ends of the leucine zippers and are used to bind together the assembly unit. However, in other embodiments cysteine residues are placed at the border of any domain within the assembly unit. In certain embodiments, such added cysteine residues are flanked or bracketed by one or more, preferably two to five, glycine residues.

Dimer formation by leucine zippers is a cooperative process, and, therefore, the length of the leucine zipper affects the stability of the binding between two helices (Su et al., 1994, Effect of chain length on the formation and stability of synthetic α-helical coiled coils, Biochemistry 33: 15501-10).There is a significant increase in temperature stability between three and four heptads but a lesser increase for longer helices. In certain embodiments of the invention, four heptads can be used for a single uninterrupted unit dimerization region, while two three-heptad regions will be required when the functional sequence interrupts the heptad (see below).

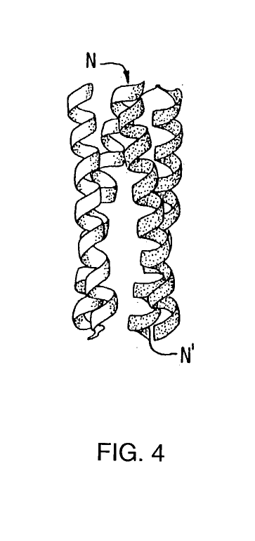

Structural elements comprising four-helix bundles can also be employed in assembly units in the nanaostructures of the invention. The design and construction of leucine zippers represent one type of a coiled coil oligomerization peptide useful in the construction of a structural element of an assembly unit. Another type is a four-helix bundle, a non-limiting example of which is shown in FIG. 4. Because there are one or more loop segments (i.e. non-helical segments) joining the helices to form an assembly unit, this structure is also called a “helix-loop-helix” structure. The loop sections contribute to the stability of the overall structure by keeping the helices near each other and, therefore, at a functionally high concentration. Examples of helix-loop-helix proteins include, but are not limited to: the bacterial Rop protein (a homodimer containing two helix-loop-helix molecules) (Lassalle et al., 1998, Dimer-to-tetramer transformation: loop excision dramatically alters structure and stability of the ROP four alpha-helix bundle protein, J. Mol. Biol. 279(4): 987-1000); the eukaryotic cytochrome b562 (a monomeric protein made up of a single helix-loop-helix-loop-helix-loop-helix structure) (Lederer et al., 1981, Improvement of the 2.5 Å resolution model of cytochrome b562 by redetermining the primary structure and using molecular graphics, J. Mol. Biol. 148(4): 427-48); Max (Lavigne et al., 1998, Insights into the mechanism of heterodimerization from the 1H-NMR solution structure of the c-Myc-Max heterodimeric leucine zipper, J. Mol. Biol. 281(1): 165-81); MyoD DNA-binding domain (Ma et al., 1994, Crystal structure of MyoD bHLH domain-DNA complex: perspectives on DNA recognition and implications for transcriptional activation, Cell 77(3): 451-59); USF1 and USF2 DNA-binding domains (Ferre-D'Amare et al., 1994, Structure and function of the b/HLH/Z domain of USF, EMBO J. 13(1): 180-9; Kurschner et al, 1997, USF2/FIP associates with the b-Zip transcription factor, c-Maf, via its bHLH domain and inhibits c-Maf DNA binding activity, Biochem. Biophys. Res. Commun. 231(2): 333-39); and Mit-f transcription factor DNA-binding domains (Rehli et al., 1999, Cloning and characterization of the murine genes for bHLH-ZIP transcription factors TFEC and TFEB reveal a common gene organization for all MiT subfamily members, Genomics 56(1): 111-20).

Both helical regions and loop regions of the Rop protein exhibit properties that indicate that the Rop protein, or fragments thereof, may be used as structural elements in the construction of assembly units in the staged assembly methods of the invention. In one embodiment, the methods of Munson et al. (1996, What makes a protein a protein? Hydrophobic core designs that specify stability and structural properties, Protein Science 5: 1584-93) are used to mutagenize the a and d residues in the helical regions of the Rop protein to produce variant polypeptides having both increased and decreased thermal stability.

In another embodiment, the methods of Betz et al. (1997, De novo design of native proteins: Characterization of proteins intended to fold into antiparallel, Rop-like, four-helix bundles, Biochemistry 36: 2450-58) are used to design synthetic 55-residue proteins that are based on the Rop protein and that form dimers in the predicted anti-parallel arrangement.

Assembly units for staged assembly based on a Rop protein-like four-helix bundle are constructed with synthetic proteins and oligopeptides including, but not limited to, those of Betz et al. (1997, De novo design of native proteins: Characterization of proteins intended to fold into antiparallel, Rop-like, four-helix bundles, Biochemistry 36: 2450-58). As disclosed in Betz and DeGrado (1996, Controlling topology and native-like behavior of de novo-designed peptides: design and characterization of antiparallel four-stranded coiled coils, Biochemistry 35: 6955-62) and Betz et al. (1997, De novo design of native proteins: Characterization of proteins intended to fold into antiparallel, Rop-like, four-helix bundles, Biochemistry 36: 2450-58), synthetic four-helix bundles can be made from two peptides that have the general form of:

Ncap-(AₐZ_{b}Z_{c}L_{d}YₑZ_{f}Y_{g})₃-Turn-(XₐZ_{b}Z_{c}L_{d}YₑZ_{f}Y_{g})₃-Ccap-CONH₂

where the a-g subscripts refer to heptad position, X is either alanine or valine, Y is glutamic acid, arginine, tyrosine or lysine, Z is any amino acid, Ncap and Ccap are alpha-helix ending residues as defined by Richardson and Richardson (1988, Amino acid preferences for specific locations at the ends of alpha helices, Science 240: 1648-52) and turns are 3-5 glycines.

In certain embodiments, PNA sequences are added to the amino terminus of one assembly unit and the carboxy terminus of the other assembly unit. This leaves the other two ends of the molecules, as well as the loop regions, available for the insertion of one or more functional elements. Proper folding of such four-helix bundles can be monitored by CD spectroscopy, ELISA analysis of the constructed assembly unit, and by electron microscopic analysis of the assembly unit and/or nanostructure fabricated from such assembly units.

The PNA assembly unit of the present invention includes at least one PNA as a joining or functional element. Additional joining elements in the PNA assembly unit may be any joining element that confers binding properties on the assembly unit including, but not limited to: pilin joining elements, haptens, antigens, peptides, PNAs, DNAs, RNAs, aptamers, polymers or other moieties, or combination thereof, that can interact through specific non-covalent interactions, with another joining element.

In certain embodiments, an assembly unit having more than two joining elements is used to build a nanostructure. The additional joining elements can be used, for example: (i) as an attachment point for addition or insertion of a functional element or functional moiety; (ii) as the attachment point of the initiator to a solid substrate; or (iii) as attachment points for subassemblies.

The PNA assembly unit used in the invention may also incorporate functional elements. These may be in the form of PNA functional elements, as described above, or they may be non-PNA functional elements. Functional elements may be incorporated into assembly units and, ultimately into one-, two-, and three-dimensional nanostructures in such a manner as to provide well-defined spatial relationships between and among the functional elements. These well-defined spatial relationships between and among the functional elements permit them to act in concert to provide activities and properties that are not attainable individually or as unstructured mixtures.

In one aspect of the invention, functional elements include, but are not limited to, peptides, proteins, protein domains, small molecules, inorganic nanoparticles, atoms, clusters of atoms, magnetic, photonic or electronic nanoparticles. The specific activity or property associated with a particular functional element, which will generally be independent of the structural attributes of the assembly unit to which it is attached, can be selected from a very large set of possible functions, including but not limited to, a biological property such as those conferred by proteins (e.g., a transcriptional, translational, binding, modifying or catalyzing property). In other embodiments, functional groups may be used that confer chemical, organic, physical electrical, optical, structural, mechanical, computational, magnetic or sensor properties to the assembly unit.

In another aspect of the invention, functional elements include, but are not limited to: metallic or metal oxide nanoparticles (Argonide Corporation, Sanford, Fla.; NanoEnergy Corporation, Longmont, Colo.; Nanophase Technologies Corporation, Romeoville, Ill.; Nanotechnologies, Austin, Tex.; TAL Materials, Inc., Ann Arbor, Mich.); gold or gold-coated nanoparticles (Nanoprobes, Inc., Yaphank, N.Y.; Nanospectra LLC, Houston Tex.); immunoconjugates (Nanoprobes, Inc., Yaphank, N.Y.); non-metallic nanoparticles (Nanotechnologies, Austin, Tex.); ceramic nanofibers (Argonide Corporation, Sanford, Fla.); fullerenes or nanotubes (e.g., carbon nanotubes) (Materials and Electrochemical Research Corporation, Tucson, Ariz.; Nanolab, Brighton Mass.; Nanosys, Inc., Cambridge Mass.; Carbon Nanotechnologies Incorporated, Houston, Tex.); nanocrystals (NanoGram Corporation, Fremont, Calif.; Quantum Dot Corporation, Hayward Calif.); silicon or silicate nanocrystals or powders (MTI Corporation, Richmond, Calif.); nanowires (Nanosys, Inc., Cambridge Mass.); or quantum dots (Quantum Dot Corporation, Hayward Calif.; Nanosys, Inc., Cambridge Mass.).

Functional elements may also comprise any art-known detectable marker, including radioactive labels such as ³²P, ³⁵S, ³H, and the like; chromophores; fluorophores; chemiluminescent molecules; or enzymatic markers.

In certain embodiment of this invention, a functional element is a fluorophore. Exemplary fluorophore moieties that can be selected as labels are set forth in Table 5.

TABLE 5
Fluorophore Moieties That Can Be Used as Functional Elements

4-acetamido-4′-isothiocyanatostilbene-2,2′disulfonic acid
acridine and derivatives:

acridine
acridine isothiocyanate

5-(2′-aminoethyl)aminonaphthalene-1-sulfonic acid (EDANS)
4-amino-N-[3-vinylsulfonyl)phenyl]naphthalimide-3,5 disulfonate (Lucifer Yellow VS) -
(4-anilino-1-naphthyl)maleimide
anthranilamide
Brilliant Yellow
coumarin and derivatives:

coumarin
7-amino-4-methylcoumarin (AMC, Coumarin 120)
7-amino-4-trifluoromethylcoumarin (Coumarin 151)

Cy3
Cy5
cyanosine
4′,6-diaminidino-2-phenylindole (DAPI)
5′,5″-dibromopyrogallol-sulfonephthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansyl chloride)
4-(4′-dimethylaminophenylazo)benzoic acid (DABCYL)
4-dimethylaminophenylazophenyl-4′-isothiocyanate (DABITC)
eosin and derivatives:

eosin
eosin isothiocyanate

erythrosin and derivatives:

erythrosin B
erythrosin isothiocyanate

ethidium
fluorescein and derivatives:

5-carboxyfluorescein (FAM)
5-(4,6-dichlorotriazin-2-yl)aminofluorescein (DTAF)
2′7′-dimethoxy-4′5′-dichloro-6-carboxyfluorescein (JOE)
fluorescein
fluorescein isothiocyanate
QFITC (XRITC)

fluorescamine
IR144
IR1446
Malachite Green isothiocyanate
4-methylumbelliferone
ortho cresolphthalein
nitrotyrosine
pararosaniline
Phenol Red
B-phycoerythrin
o-phthaldialdehyde
pyrene and derivatives:

pyrene
pyrene butyrate
succinimidyl 1-pyrene butyrate

Reactive Red 4 (Cibacron ® Brilliant Red 3B-A)
rhodamine and derivatives:

6-carboxy-X-rhodamine (ROX)
6-carboxyrhodamine (R6G)
lissamine rhodamine B sulfonyl chloride
rhodamine (Rhod)
rhodamine B
rhodamine 110
rhodamine 123
rhodamine X isothiocyanate
sulforhodamine B
sulforhodamine 101
sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)

riboflavin
rosolic acid
terbium chelate derivatives

In other embodiments, a functional element is a chemiluminescent substrate such as luminol (Amersham Biosciences), BOLD™ APB (Intergen), Lumigen APS (Lumigen), etc.

In another embodiment, the functional element is an enzyme. The enzyme, in certain embodiments, may produce a detectable signal when a particular chemical reaction is conducted, such as the enzymes alkaline phosphatase, horseradish peroxidase, β-galactosidase, etc.

In another embodiment, a functional element is a hapten or an antigen (e.g., ras). In yet another embodiment, a functional element is a molecule such as biotin, to which a labeled avidin molecule or streptavidin may be bound, or digoxygenin, to which a labeled anti-digoxygenin antibody may be bound.

In another embodiment, a functional element is a lectin such as peanut lectin or soybean agglutinin. In yet another embodiment, a functional element is a toxin, such as Pseudomonas exotoxin (Chaudhary et al., 1989, A recombinant immunotoxin consisting of two antibody variable domains fused to Pseudomonas exotoxin, Nature 339(6223): 394-97).

Peptides, proteins or protein domains may be added to proteinaceous assembly units using the tools of molecular biology commonly known in the art to produce fusion proteins in which the functional elements are introduced at the N-terminus of the proteins, the C-terminus of the protein, or in a loop within the protein in such a way as to not disrupt folding of the protein. Non-peptide functional elements may be added to an assembly unit by the incorporation of a peptide or protein moiety that exhibits specificity for said functional element, into the proteinaceous portion of the assembly unit.

In a specific embodiment, one or more functional elements is added to an assembly unit comprising a pilin protein at a position identified as being (i) on the surface of the unit; (ii) unimportant to the interaction of the unit with other pilin-comprising assembly unit; and (iii) unimportant for the stability of the unit itself. It has been shown that large loop insertions are tolerated and many recombinant proteins have been expressed that are able to fold successfully into stable, active protein structures. In some instances, such recombinant proteins have been designed and produced without further genetic manipulation, while other approaches have incorporated a randomization and selection step to identify optimal sequence alterations (Regan, 1999, Protein redesign, Curr. Opin. Struct. Biol. 9: 494-99). For example, one pilin region amenable to re-engineering is a surface loop on papA comprising the sequence gly107-ala108-gly109. This loop satisfies all the above-described criteria as a position at which a heterologous peptide may be inserted.

In another embodiment, an entire antibody variable domain (e.g. a single-chain variable domain) is incorporated into an assembly unit, e.g. into the joining or structural element thereof, in order to act as an affinity target for a functional element. In this embodiment, wherein an entire antibody variable domain is inserted into a surface loop of, e.g., a joining element or a structural element, a flexible segment (e.g., a polyglycine peptide sequence) is preferably added to each side of the variable domain sequence. This polyglycine linker will act as a flexible spacer that facilitates folding of the original protein after synthesis of the recombinant fusion protein. The antibody domain is chosen for its binding specificity for a functional element, which can be, but is not limited to, a protein or peptide, or to an inorganic material.

In another embodiment of the present invention, a functional element may be a quantum dot (semiconductor nanocrystal, e.g., QDOT™, Quantum Dot Corporation, Hayward, Calif.) with desirable optical properties. A quantum dot can be incorporated into a nanostructure through a peptide that has specificity for a particular class of quantum dot. As would be apparent to one of ordinary skill, identification of such a peptide, having a required affinity for a particular type of quantum dot, is carried out using methods well known in the art. For example, such a peptide is selected from a large library of phage-displayed peptides using an affinity purification method. Suitable purification methods include, e.g., biopanning (Whaley et al., 2000, Selection of peptides with semiconductor binding specificity for directed nanocrystal assembly, Nature 405(6787): 665-68) and affinity column chromatography. In each case, target quantum dots are immobilized and the recombinant phage display library is incubated against the immobilized quantum dots. Several rounds of biopanning are carried out and phage exhibiting affinity for the quantum dots are identified by standard methods after which the specificity of the peptides are tested using standard ELISA methodology.

Typically, the affinity purification is an iterative process that uses several affinity purification steps. Affinity purification may been used to identify peptides with affinity for particular metals and semiconductors (Belcher, 2001, Evolving Biomolecular Control of Semiconductor and Magnetic Nanostructure, presentation at Nanoscience: Underlying Physical Concepts and Properties, National Academy of Sciences, Washington, D.C., May 18-20, 2001; Belcher et al., 2001, Abstracts of Papers, 222nd ACS National Meeting, Chicago, Ill., United States, Aug. 26-30, 2001, American Chemical Society, Washington, D.C.).

An alternate method is directed toward the use of libraries of phage-displayed single chain variable domains, and to carry out the same type of selection process. Accordingly, in certain embodiments, a functional element is incorporated into a nanostructure through the use of joining elements (interaction sites) by which non-proteinaceous nanoparticles having desirable properties are attached to the nanostructure. Such joining elements are, in two non-limiting examples, derived from the complementarity determining regions of antibody variable domains or from affinity selected peptides.

Routine tests for electronic and photonic functional elements that are commonly used to compare the electronic properties of nanocrystals (single nanoparticles) and assemblies of nanoparticles (Murray et al., 2000, Synthesis and characterization of monodisperse nanocrystals and close-packed nanocrystal assemblies, Ann. Rev. Material Science 30: 545-610), are used for the analysis of nanostructures fabricated using the compositions and methods disclosed herein.

In certain embodiments, the unique, tunable properties of semiconductor nanocrystals make them preferable for use in nanodevices, including photoconductive nanodevices and light emitting diodes. The electrical properties of an individual nanostructure are difficult to measure, and therefore, photoconductivity is used as a measure of the properties of those nanostructures. Photoconductivity is a well-known phenomena used for analysis of the properties of semiconductors and organic solids. Photoconductivity has long been used to transport electrons between weakly interacting molecules in otherwise insulating organic solids.

Photocurrent spectral responses may also be used to map the absorption spectra of the nanocrystals in nanostructures and compared to the photocurrent spectral responses of individual nanocrystals (see, e.g., Murray et al., 2000, Synthesis and characterization of monodisperse nanocrystals and close-packed nanocrystal assemblies, Ann. Rev. Material Science 30: 545-610). In addition, optical and photoluminescence spectra may also be used to study the optical properties of nanostructures (see, e.g., Murray et al., 2000, Synthesis and characterization of monodisperse nanocrystals and close-packed nanocrystal assemblies, Ann. Rev. Material Science 30: 545-610).

The greater the control exerted over the formation of arrays of nanoparticles, the wider the array of optical, electrical and magnetic phenomena that will be produced. With staged assembly of nanostructures into which nanoparticles are incorporated with three-dimensional precision, it is possible to control the properties of solids formed therefrom in three dimensions, thereby giving rise to a host of anisotropic properties useful in the design of nanodevices. Routine tests and methods for characterizing the properties of these assemblages are well-known in the art (see, e.g., Murray et al., 2000, Synthesis and characterization of monodisperse nanocrystals and close-packed nanocrystal assemblies, Ann. Rev. Material Sci. 30: 545-610).

For example, biosensors are commercially available that are made of a combination of proteins and quantum dots (Alivisatos et al., 1996, Organization of ‘nanocrystal molecules’ using DNA, Nature 382: 609-11; Weiss et al., U.S. Pat. No. 6,207,392 entitled “Semiconductor nanocrystal probes for biological applications and process for making and using such probes,” issued Mar. 27, 2001). The ability to complex a quantum dot with a highly specific biological molecule (e.g., a single stranded DNA or an antibody molecule) provides a spectral fingerprint for the target of the molecule. Using different sized quantum dots (each with very different spectral properties), each complexed to a molecule with different specificity, allows multiple sensing of components simultaneously.

Inorganic structures such as quantum dots and nanocrystals of metals or semiconductors may be used in the staged assembly of nanostructures as termini of branches of the assembled nanostructure. Once such inorganic structures are added, additional groups cannot be attached to them because they have an indeterminate stoichiometry for any set of binding sites engineered into a nanostructure. This influences the sequence in which assembly units are added to form a nanostructure being fabricated by staged assembly. For example, once a particular nanocrystal is added to the nanostructure, it is generally not preferred to add additional assembly units with joining elements that recognize and bind that type of nanocrystal, because it is generally not possible to control the positioning of such assembly units relative to the nanocrystal. Therefore, it may be necessary to add the nanocrystals last, or at least after all the assembly units that will bind that particular type of nanocrystal are added. In a preferred embodiment, nanocrystals are added to nanostructures that are still bound to a matrix and are sufficiently separated so that each nanocrystal can only bind to a single nanostructure, thereby preventing multiple cross-linking of nanostructures.

In one embodiment, a rigid nanostructure, fabricated according to the staged assembly methods of the present invention, comprises a magnetic nanoparticle attached as a functional element to the end of a nanostructure lever arm, which acts as a very sensitive sensor of local magnetic fields. The presence of a magnetic field acts to change the position of the magnetic nanoparticle, bending the nanostructure lever arm relative to the solid substrate to which it is attached. The position of the lever arm may be sensed, in certain embodiments, through a change in position of, for example, optical nanoparticles attached as functional elements to other positions (assembly units) along the nanostructure lever arm. The degree of movement of the lever arm is calibrated to provide a measure of the magnetic field.

In other embodiments, nanostructures that are fabricated according to the staged assembly methods of the invention have desirable properties in the absence of specialized functional elements. In such embodiments, a staged assembly process provides a two-dimensional or a three-dimensional nanostructure with small (nanometer-scale), precisely-sized, and well-defined pores that can be used, for example, for filtering particles in a microfluidic system. In further aspects of this embodiment, nanostructures are assembled that not only comprise such well-defined pores but also comprise functional elements that enhance the separation properties of the nanostructure, allowing separations based not only on size but also with respect to the charge and/or hydrophilicity or hydrophobicity properties of the molecules to be separated. Such nanostructures can be used for HPLC separations, providing extremely uniform packing materials and separations based upon those materials. Examples of such functional elements include, but are not limited to, peptide sequences comprising one or more side chains that are positively or negatively charged at a pH used for the desired chromatographic separation; and peptide sequences comprising one or more amino acids having hydrophobic or lipophilic side chains.

Junctions are architectural structures that can serve as “switch points” in microelectronic circuits such as silicon based electronic chips, etc. In certain embodiments, multivalent antibodies or binding derivatives or binding fragments thereof are used as junction structures and are introduced into nanostructures using the methods of the present invention. One non-limiting example of bioelectronic and biocomputational devices comprising these nanostructure junctions are quantum cellular automata (QCA).

Staged Assembly of Nanostructures

PNA assembly units may be assembled to form nanostructures by staged assembly. Staged assembly enables massively parallel synthesis of complex, non-periodic, multi-dimensional nanostructures in which organic and inorganic moieties are placed, accurately and precisely, into a pre-designed, three-dimensional architecture. In a staged assembly, a series of assembly units is added in a given pre-designed order to an initiator unit and/or nanostructure intermediate. Because a large number of identical initiators are used and because subunits are added to all initiators/intermediates simultaneously, staged assembly fabricates multiple identical nanostructures in a massively parallel manner. In preferred embodiments, the initiator units are bound to a solid substrate, support or matrix. Additional assembly units are added sequentially in a procedure akin to solid phase polymer synthesis. The intermediate stage(s) of the nanostructure while it is being assembled, and which comprises the bound assembly units formed on the initiator unit, is generally described as either a nanostructure intermediate or simply, a nanostructure. Addition of each assembly unit to the nanostructure intermediate undergoing assembly depends upon the nature of the joining element presented by the previously added assembly unit and is independent of subsequently added assembly units. Thus assembly units can bind only to the joining elements exposed on the nanostructure intermediate undergoing assembly; that is, the added assembly units do not self-interact and/or polymerize.

Since the joining elements of a single assembly unit are non-complementary and therefore do not interact with one another, unbound assembly units do not form dimers or polymers. An assembly unit to be added is preferably provided in molar excess over the initiator unit or nanostructure intermediate in order to drive its reaction with the intermediate to completion. Removal of unbound assembly units during staged assembly is facilitated by carrying out staged assembly using a solid-substrate-bound initiator so that unbound assembly units can be washed away in each cycle of the assembly process.

This scheme provides for assembly of complex nanostructures using relatively few non-cross-reacting, complementary joining pairs. Only a few joining pairs need to be used, since only a limited number of joining elements will be exposed on the surface of an assembly intermediate at any one step in the assembly process. Assembly units with complementary joining elements can be added and incubated against the nanostructure intermediate, causing the added assembly units to be attached to the nanostructure intermediate during an assembly cycle. Excess assembly units can then be washed away to prevent them from forming unwanted interactions with other assembly units during subsequent steps of the assembly process. Each position in the nanostructure can be uniquely defined through the process of staged assembly and distinct functional elements can be added at any desired position. The staged assembly method of the invention enables massive parallel manufacture of complex nanostructures, and different complex nanostructures can be further self-assembled into higher order architectures in a hierarchic manner.

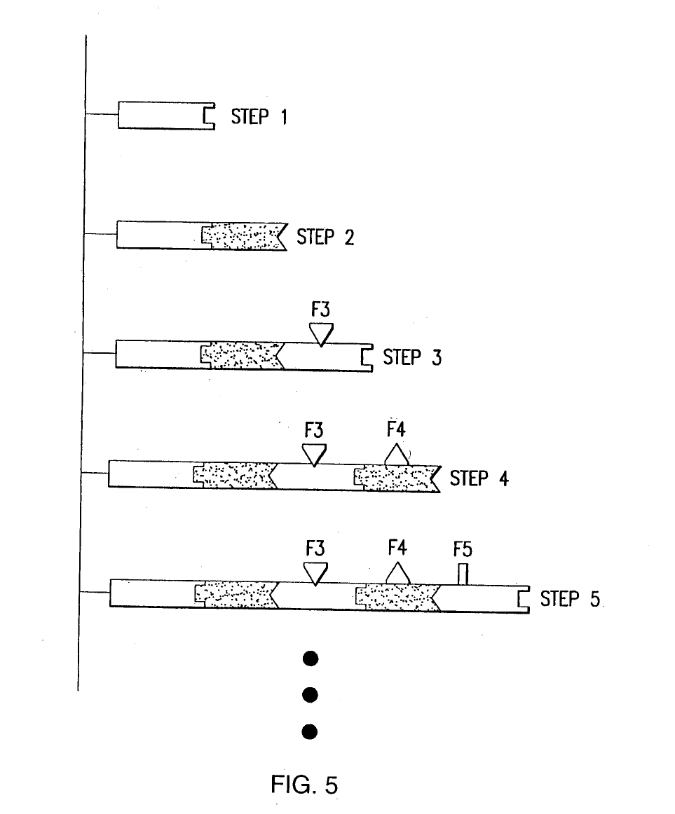

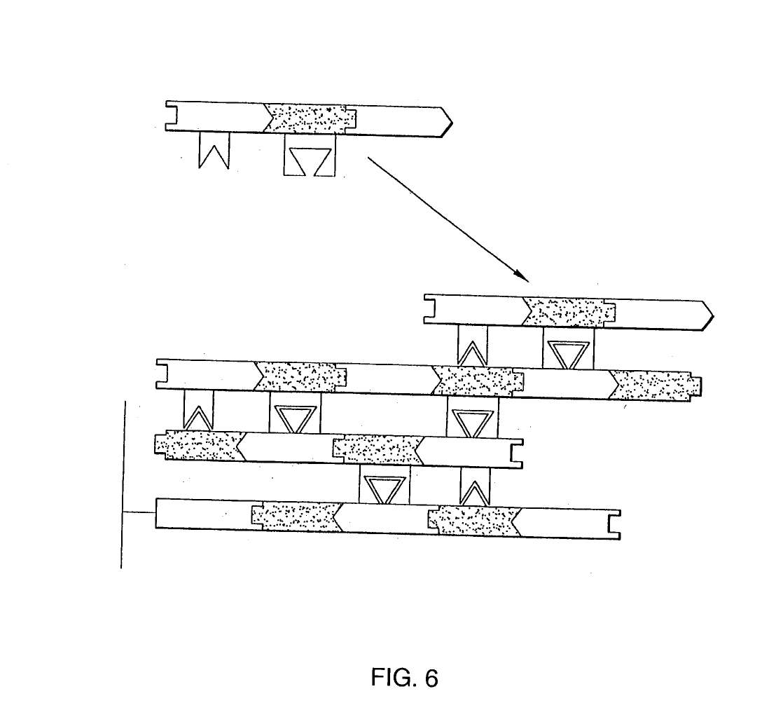

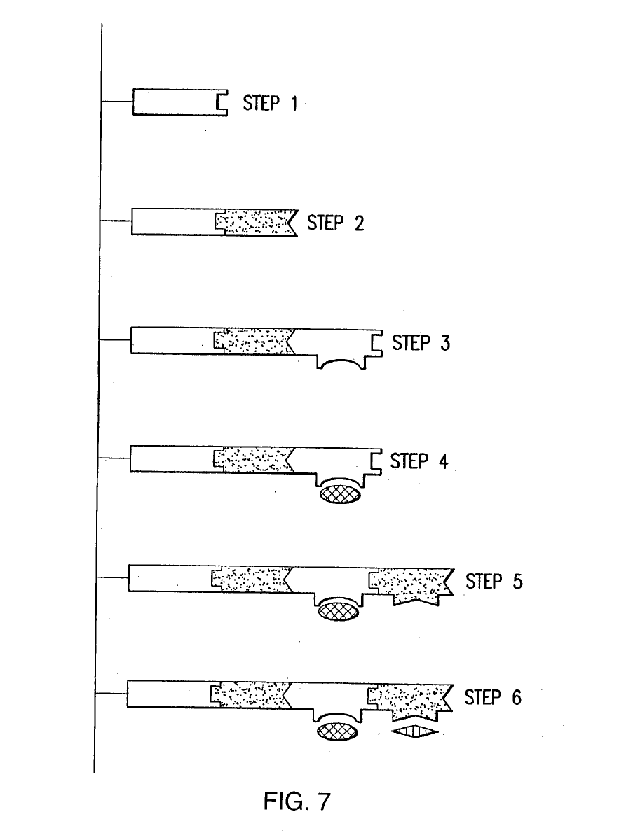

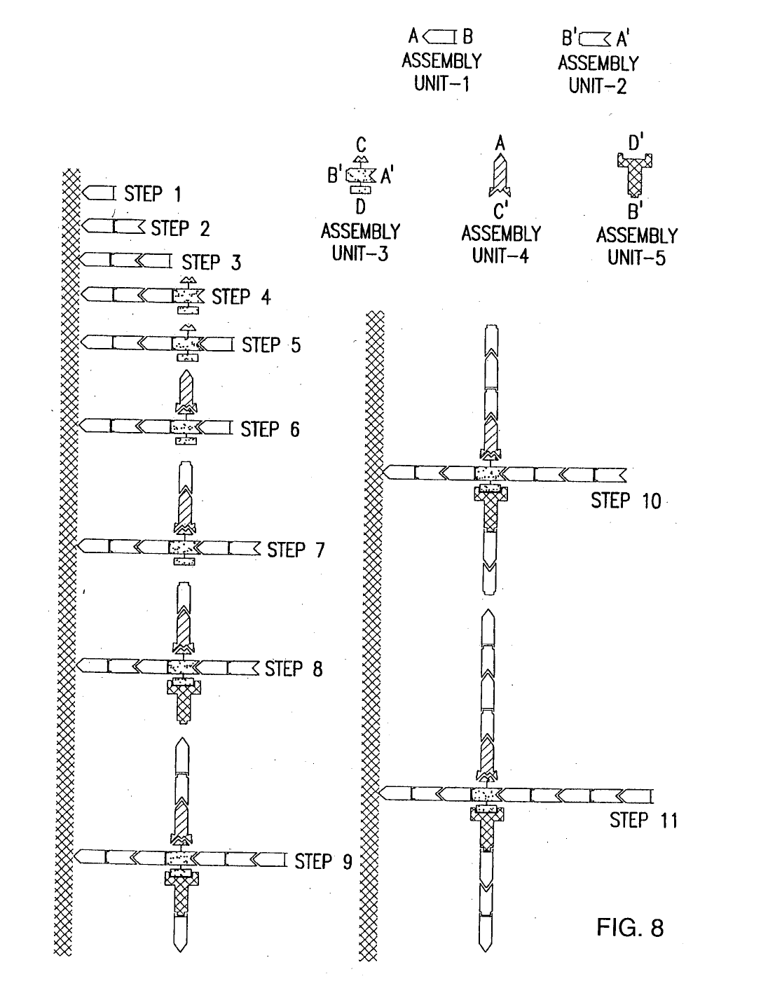

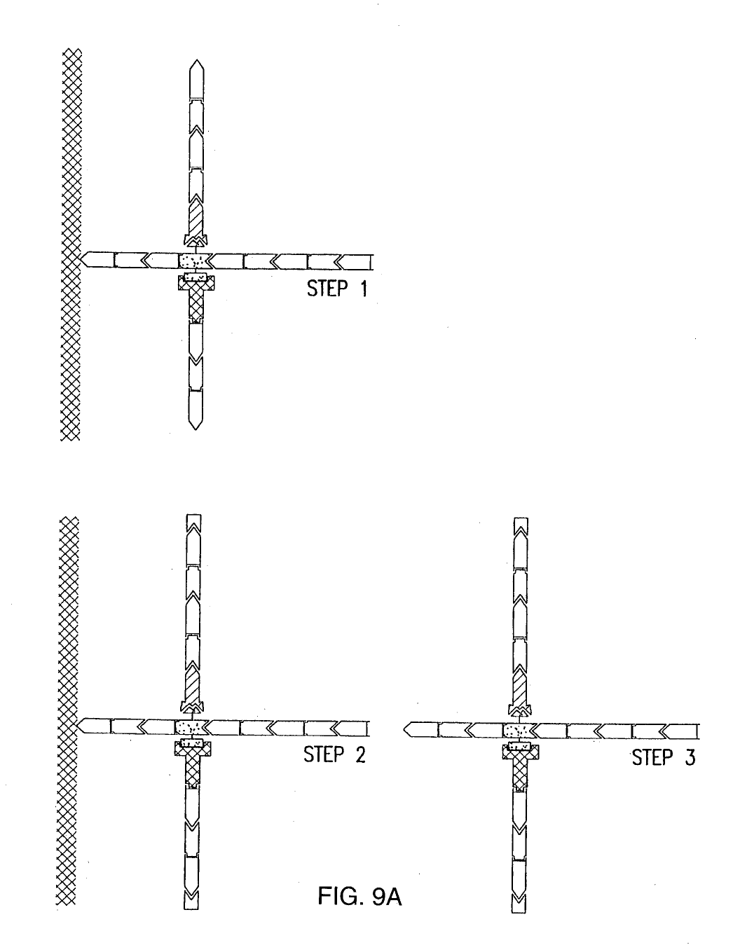

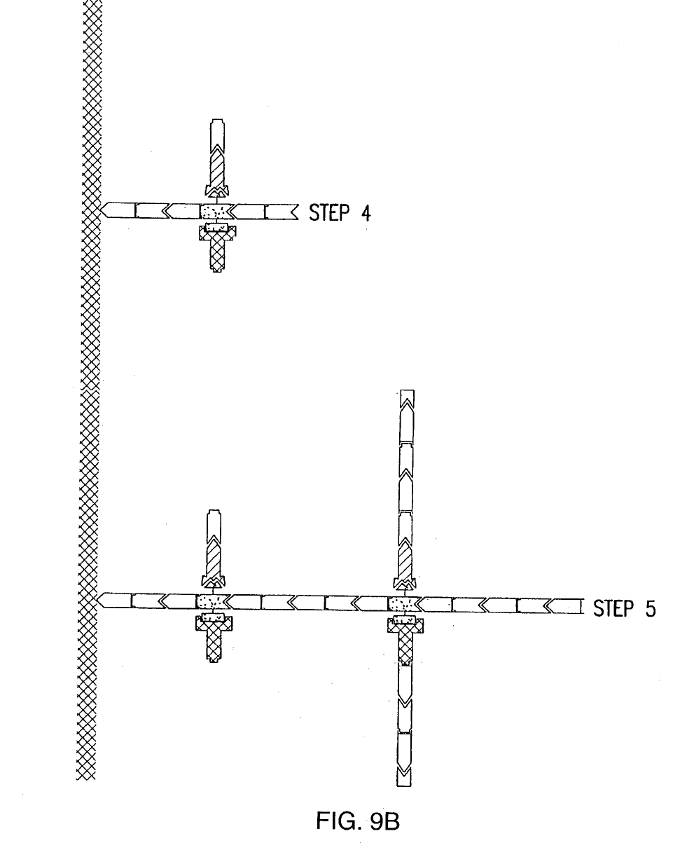

FIG. 5 depicts an embodiment of the staged assembly method of the invention in one dimension. In step 1, an initiator unit is immobilized on a solid substrate. In step 2, an assembly unit is added to the initiator (i.e. the matrix bound initiator unit), resulting in a nanostructure intermediate composed of two units. Only a single assembly unit is added in this step, because the second assembly unit cannot interact (i.e. polymerize) with itself.

The initiator unit, or any of the assembly units subsequently added during staged assembly including the capping unit, may contain an added functional element and/or may comprise a structural unit of different length from previously added units. For example, in step 3 of FIG. 5, a third assembly unit is added that comprises a functional element. In steps 4 and 5, additional assembly units are added, each with a designed functional group. Thus in the embodiment of staged assembly depicted in FIG. 5, the third, fourth and fifth assembly units each carry a unique functional element (designated by geometric shapes protruding from the top of the assembly units in the figure).

The embodiment of staged assembly depicted in FIG. 5 requires only two non-cross-reacting, complementary joining pairs. Self-assembly of the structure, as it stands at the end of step 5, would require four non-cross-reacting, complementary joining pairs. This relatively modest improvement in number of required joining pairs becomes far greater as the size of the structure increases. For instance, for a linear structure of N units assembled by an extension of the five steps illustrated in FIG. 5, staged assembly would still require only two non-cross-reacting, complementary joining pairs, whereas self-assembly would require (N−1) non-cross-reacting, complementary joining pairs.

The number of nanostructures fabricated is determined by the number of initiator units bound to the matrix while the length of each one-dimensional nanostructure is a function of the number of assembly cycles performed. If assembly units with one or more different functional elements are used, then the order of assembly will define the relative spatial orientation of each functional element relative to the other functional elements.

After each step in the method of staged assembly of the invention, excess unbound assembly units are removed from the attached nanostructure intermediate by a removal step, e.g., a washing step. The substrate-bound nanostructure intermediate may be washed with an appropriate solvent (e.g., an aqueous solution or buffer). The solvent must be able to remove subunits held by non-specific interactions without disrupting the specific, interactions of complementary joining elements. Appropriate solvents may vary as to pH, salt concentration, chemical composition, etc., as required by the assembly units being used.

A buffer used for washing the nanostructure intermediate can be, for example, a buffer used in the wash steps implemented in ELISA protocols, such as those described in Current Protocols in Immunology (see Chapter 2, Antibody Detection and Preparation, Section 2.1 “Enzyme-Linked Immunosorbent Assays,” John Wiley & Sons, 2001, Editors John E. Coligan, Ada M. Kruisbeek, David H. Margulies, Ethan M. Shevach, Warren Strober, Series Editor: Richard Coico).

In certain embodiments, an assembled nanostructure is “capped” by addition of a “capping unit,” which is an assembly unit that carries only a single joining element. Furthermore, if the initiator unit has been attached to the solid substrate via a cleavable bond, the nanostructure can be removed from the solid substrate and isolated. However, in some embodiments, the completed nanodevice will be functional while attached to the solid substrate and need not be removed.

The above-described steps of adding assembly units can be repeated in an iterative manner until a complete nanostructure is assembled, after which time the complete nanostructure can be released by breaking the bond immobilizing the first assembly unit from the matrix at a designed releasing moiety (e.g,. a protease site) within the initiator unit or by using a pre-designed process for release (e.g., lowering of pH). The process of staged assembly, as illustrated in FIGS. 2 and 3 is one of the simplest embodiments contemplated for staged assembly. In other embodiments, assembly units with additional joining elements can be used to create more complex assemblies. Assembly units may be added individually or, in certain embodiments, they can be added as subassemblies (FIG. 6). The result is a completely defined nanostructure with functional elements that are distributed spatially in relationship to one another to satisfy desired design parameters. The compositions and methods disclosed herein provide means for the assembly of these complex, designed nanostructures and of more complex nanodevices formed by the staged assembly of one or a plurality of nanostructures into a larger structure. Fabrication of multidimensional nanostructures can be accomplished, e.g., by incorporating precisely-spaced assembly units containing additional joining elements into individual, one-dimensional nanostructures, where those additional joining elements can be recognized and bound by a suitable cross-linking agent to attach the individual nanostructures together. In certain preferred embodiments, such cross-linking could be, e.g., an antibody or a binding derivative or a binding fragment thereof.

In some embodiments of the staged assembly method of the invention, the initiator unit is tethered to a solid support. Such tethering is not random (i.e., is not non-specific binding of protein to plastic or random biotinylation of an assembly unit followed by binding to immobilized streptavidin) but involves the binding of a specific element of the initiator unit to the matrix or substrate. The staged assembly process is a vectorial process that requires an unobstructed joining element on the initiator unit for attachment of the next assembly unit. Random binding of initiator units to substrate would, in some cases, result in the obstruction of the joining element needed for the attachment of the next assembly unit, and thus lowering the number of initiator units on which nanostructures are assembled.

In other embodiments of the staged assembly method of the invention, the initiator unit is not immobilized to a solid substrate. In this case, a removal step, e.g., a washing step, can be carried out on a nanostructure constructed on a non-immobilized or untethered initiator unit by: (1) attaching a magnetic nanoparticle to the initiator unit and separating nanostructure intermediates from non-bound assembly units by applying a magnetic field; 2) separating the larger nanostructure intermediates from unbound assembly units by centrifugation, precipitation or filtration; or 3) in those instances in which a nanostructure intermediate or assembled nanostructure is more resistant to a destructive treatment (e.g., protease treatment or chemical degradation), unbound assembly units are selectively destroyed.

Proteins have well-defined binding properties, and the technology to manipulate the intermolecular interactions of proteins is well known in the art (Hayashi et al., 1995, A single expression system for the display, purification and conjugation of single-chain antibodies, Gene 160(1): 129-30; Hayden et al, 1997, Antibody engineering, Curr. Opin. Immunol. 9(2): 201-12; Jung et al., 1999, Selection for improved protein stability by phage display, J. Mol. Biol. 294(1): 163-80, Viti et al., 2000, Design and use of phage display libraries for the selection of antibodies and enzymes, Methods Enzymol. 326: 480-505; Winter et al., 1994, Making antibodies by phage display technology, Annu. Rev. Immunol. 12: 433-55). The contemplated staged assembly of nanostructures, however, need not be limited to components composed primarily of biological molecules, e.g., proteins and nucleic acids, that have specific recognition properties. The optical, magnetic or electrical properties of inorganic atoms or molecules will be required for some embodiments of nanostructures fabricated by staged assembly.

There will be many embodiments of this invention in which components not made up of proteins will be advantageously utilized. In other embodiments, it may be possible to utilize the molecular interaction properties of proteins or nucleic acids to construct nanostructures composed of both organic and inorganic materials.

In certain embodiments, inorganic nanoparticles are added to components that are assembled into nanostructures using the staged assembly methods of the invention. This may be done using joining elements specifically selected for binding to inorganic particles. For example, Whaley and co-workers have identified peptides that bind specifically to semiconductor binding surfaces (Whaley et al., 2000, Selection of peptides with semiconductor binding specificity for directed nanocrystal assembly, Nature 405: 665-68). In one embodiment, these peptides are inserted into protein components described herein using standard cloning techniques. Staged assembly of protein constructs as disclosed herein, provides a means of distributing these binding sites in a rigid, well-defined three-dimensional array.

Once the binding sites for a particular type of inorganic nanoparticle are all in place, the inorganic nanoparticles can be added using a cycle of staged assembly analogous to that used to add proteinaceous assembly units. To accomplish this, it may be necessary, in certain embodiments to adjust the solution conditions under which the nanostructure intermediates are incubated, in order to provide for the solubility of the inorganic nanoparticles. Once an inorganic nanoparticle is added to the nanostructure intermediate, it is not possible to add further units to the inorganic nanoparticle in a controlled fashion because of the microheterogeneities intrinsic to any population of inorganic nanoparticles. These heterogeneities would render the geometry and stoichiometry of further interactions uncontrollable.

FIG. 7 is a diagram illustrating the addition of protein units and inorganic elements to a nanostructure according to the staged assembly methods of the invention. In step 1, an initiator unit is bound to a solid substrate. In step 2, an assembly unit is bound specifically to the initiator unit. In step 3, an additional assembly unit is bound to the nanostructure undergoing assembly. This assembly unit comprises an engineered binding site specific for a particular inorganic element. In step 4, the inorganic element (depicted as a cross-hatched oval) is added to the structure and bound by the engineered binding site. Step 5 adds another assembly unit with a binding site engineered for specificity to a second type of inorganic element, and that second inorganic element (depicted as a hatched diamond) is added in step 6.

The order in which assembly units are added is determined by the desired structure and/or activity that the product nanostructure, and the need to minimize the number of cross-reacting joining element pairs used in the assembly process. Hence determining the order of assembly is an integral part of the design of a nanostructure to be fabricated by staged assembly. Joining elements are chosen, by design, to permit staged assembly of the desired nanostructure. Since the choice of joining element(s) is generally independent of the functional elements to be incorporated into the nanostructure, the joining elements are mixed and matched as needed to fabricate assembly units with the necessary functional elements and joining elements that will provide for the placement of those functional elements in the desired spatial orientation.

For example, assembly units comprising two joining elements, designed using the six joining elements that make up three joining pairs, can include any of 18 pairs of the joining elements that are non-interacting. There are 21 possible pairs of joining elements, but three of these pairs are interacting (e.g. A-A′) and their use in an assembly unit would lead to the self-association of identical assembly units with one another. In the example illustrated below, joining elements are denoted as A, A′, B, B′, C and C′, where A and A′, B and B′, and C and C′ are complementary pairs of joining elements (joining pairs), i.e. they bind to each other with specificity, but not to any of the other four joining elements depicted. Six representative assembly units, each of which comprises two joining elements, wherein each joining element comprises a non-identical, non-complementary joining element, are depicted below. In this depiction, each assembly unit further comprises a unique functional element, one of a set of six, and represented as F₁ to F₆. According to these conventions, six possible assembly units can be designated as:

A-F₁-B

B′-F₂-A′

B′-F₃-C′

C-F₄-B

B′-F₅-A′

A-F₆-C′

Staged assembly according to the methods disclosed herein can be used to assemble the following illustrative linear, one-dimensional nanostructures, in which the order and relative vectorial orientation of each assembly unit is independent of the order of the functional elements (the symbol ●- is used to represent the solid substrate to which the initiator is attached and a double colon represents the specific interaction between assembly units):

●-A-F1-B::B′-F2-A′::A-F1-B::B′-F2-A′::A-F1-B::B′-F2-A′::A-F1-B::B′-F2-A′

●-A-F1-B::B′-F2-A′::A-F6-C′::C-F4-B::B′-F2-A′::A-F1-B::B′-F5-A′::A-F6-C′

●-A-F1-B::B′-F2-A′::A-F1-B::B′-F5-A′::A-F1-B::B′-F2-A′::A-F1-B::B′-F3-C′

●-A-F1-B::B′-F3-C′::C-F4-B::B′-F3-C′::C-F4-B::B′-F3-C′::C-F4-B::B′-F2-A′

As is apparent from this illustration, a large number of unique assembly units can be constructed using a small number of complementary joining elements. Moreover, only a small number of complementary joining elements are required for the fabrication of a large number of unique and complex nanostructures, since only one type of assembly unit is added in each staged assembly cycle and, therefore, joining elements can be used repeatedly without rendering ambiguous the position of an assembly unit within the completed nanostructure.

In each of the cases illustrated above, only two or three joining pairs have been used. Self-assembly of any of these structures would require the use of seven non-cross-reacting joining pairs. If these linear structures were N units in extent and assembled using staged assembly, they would still only require two or three joining pairs, but for self-assembly, they would require (N−1) non-cross-reacting, complementary joining pairs.

In another aspect of the invention, by interchanging the positions of the two joining elements of an assembly unit depicted above, the spatial position and orientation of the attached functional element will be altered within the overall structure of the nanostructure fabricated. This aspect of the invention illustrates yet another aspect of the design flexibility provided by staged assembly of nanostructures as disclosed herein.

Attachment of each assembly unit to an initiator or nanostructure intermediate is mediated by formation of a specific joining-pair interaction between one joining element of the assembly unit and one or more unbound complementary joining elements carried by the initiator or nanostructure intermediate. In many embodiments, only a single unbound complementary joining element will be present on the initiator or nanostructure intermediate. However, in other embodiments, it may be advantageous to add multiple identical assembly units to multiple sites on the assembly intermediate that comprise identical joining elements. In these embodiments, the staged assembly proceeds by the parallel addition of assembly units, but only a single unit will be attached at any one site on the intermediate, and assembly at all sites that are involved will occur in a pre-designed, vectorial manner.

Structural integrity of the nanostructure is of critical importance throughout the process of staged assembly, and the assembly units are preferably connected by non-covalent interactions. A specific non-covalent interaction is, for example, an interaction that occurs between an assembly unit and a nanostructure intermediate. The specific interaction should exhibit adequate affinity to confer stability to the complex between the assembly unit and the nanostructure intermediate sufficient to maintain the interaction stably throughout the entire staged assembly process. A specific non-covalent interaction should exhibit adequate specificity such that the added assembly unit will form stable interactions only with joining elements designed to interact with it. The interactions that occur among elements during the staged assembly process disclosed herein are preferably operationally “irreversible.” A binding constant that meets this requirement cannot be defined unambiguously since “irreversible” is a kinetic concept, and a binding constant is based on equilibrium properties. Nevertheless, interactions with Kd's of the order of 10⁻⁷ or lower (i.e. higher affinity and similar to the Kd of a typical diabody-epitope complex) will typically act “irreversibly” on the time scale of interest, i.e. during staged assembly of a nanostructure.

The intermolecular interactions need not act “irreversibly,” however, on the timescale of the utilization of a nanostructure (i.e. its shelf life or working life expectancy). In certain embodiments, nanostructures fabricated according to the staged assembly methods disclosed herein are subsequently stabilized by chemical fixation (e.g., by fixation with paraformaldehyde or glutaraldehyde) or by cross-linking. The most common schemes for cross-linking two proteins involve the indirect coupling of an amine group on one assembly unit to a thiol group on a second assembly unit (see, e.g., Handbook of Fluorescent Probes and Research Products, Eighth Edition, Chapter 2, Molecular Probes, Inc., Eugene, Oreg.; Loster et al., 1997, Analysis of protein aggregates by combination of cross-linking reactions and chromatographic separations, J. Chromatogr. B. Biomed. Sci. Appl. 699(1-2): 439-61; Phizicky et al., 1995, Protein-protein interactions: methods for detection and analysis, Microbiol. Rev. 59(1): 94-123).

In certain embodiments of the invention, the fabrication of a nanostructure by the staged assembly methods of the present invention involves joining relatively rigid and stable assembly units, using non-covalent interactions between and among assembly units. Nevertheless, the joining elements that are incorporated into useful assembly units can be rather disordered, that is, neither stable nor rigid, prior to interaction with a second joining element to form a stable, preferably rigid, joining pair. Therefore, in certain embodiments of the invention, individual assembly units may include unstable, flexible domains prior to assembly, which, after assembly, will be more rigid. In preferred embodiments, a nanostructure fabricated using the compositions and methods disclosed herein is a rigid structure.

According to the methods of the invention, analysis of the rigidity of a nanostructure, as well as the identification of any architectural flaws or defects, are carried out using methods well-known in the art, such as electron microscopy.

In another embodiment, structural rigidity can be tested by attaching one end of a completed nanostructure directly to a solid surface, i.e., without the use of a flexible tether. The other end of the nanostructure (or a terminal branch of the nanostructure, if it is a multi-branched structure) is then attached to an atomic force microscope (AFM) tip, which is movable. Force is applied to the tip in an attempt to move it. If the nanostructure is flexible, there will be an approximately proportional relationship between the force applied and tip movement as allowed by deflection of the nanostructure. In contrast, if the nanostructure is rigid, there will be little or no deflection of the nanostructure and tip movement as the level of applied force increases, up until the point at which the rigid nanostructure breaks. At that point, there will be a large movement of the AFM tip even though no further force is applied. As long as the attachment points of the two ends are stronger than the nanostructure, this method will provide a useful measurement of rigidity.

According to the present invention, each position in a nanostructure is distinguishable from all others, since each assembly unit can be designed to interact tightly, specifically, and uniquely with its neighbors. Each assembly unit can have an activity and/or characteristic that is distinct to its position within the nanostructure. Each position in the nanostructure is uniquely defined through the process of staged assembly, and through the properties of each assembly unit and/or functional element that is added at a desired position. In addition, the staged-assembly methods and assembly units disclosed herein are amenable to large scale, massively parallel, automated manufacturing processes for construction of complex nanostructures of well-defined size, shape, and function.

The methods and compositions of the present invention capitalize upon the precise dimensions, uniformity and diversity of spatial geometries that proteins are capable of that are used in the construction of the assembly units employed herein. Furthermore, as described hereinbelow, the methods of the invention are advantageous because genetic engineering techniques can be used to modify and tailor the properties of those biological materials used in the methods of the invention disclosed herein, as well as to synthesize large quantities of such materials in microorganisms.

Initiator Assembly Units

An initiator assembly unit is the first assembly unit incorporated into a nanostructure that is formed by the staged assembly method of the invention. An initiator assembly unit may be attached, in certain embodiments, by covalent or non-covalent interactions, to a solid substrate or other matrix. An initiator assembly unit is also known as an “initiator unit.”

Staged assembly of a nanostructure begins by the non-covalent, vectorial addition of a selected assembly unit to the initiator unit. According to the methods of the invention, an assembly unit is added to the initiator unit through (i) the incubation of an initiator unit, which in some embodiments, is immobilized to a matrix or substrate, in a solution comprising the next assembly unit to be added. This incubation step is followed by (ii) a removal step, e.g., a washing step, in which excess assembly units are removed from the proximity of the initiator unit.

Assembly units bind to the initiator unit through the formation of specific, non-covalent bonds. The joining elements of the next assembly unit are chosen so that they attach only at pre-designated sites on the initiator unit. Only one assembly unit can be added to a target joining element on the initiator unit during the first staged-assembly cycle, and binding of the assembly unit to the target initiator unit is vectorial. Staged assembly continues by repeating steps (i) and (ii) until all of the desired assembly units are incorporated into the nanostructure according to the desired design of the nanostructure.

In a preferred embodiment of the staged assembly method of the invention, an initiator unit is immobilized on a substrate and additional units are added sequentially in a procedure analogous to solid phase polymer synthesis.

An initiator unit is a category of assembly unit, and therefore can comprise any of the structural, joining, and/or functional elements described hereinbelow as being comprised in an assembly unit of the invention. An initiator unit can therefore comprise any of the following molecules, or a binding derivative or binding fragment thereof: a monoclonal antibody; a multispecific antibody, a Fab or F(ab′)₂ fragment, a single-chain antibody fragment (scFv); a bispecific, chimeric or bispecific heterodimeric F(ab′)₂; a diabody or multimeric scFv fragment; a bacterial pilin protein, a leucine zipper-type coiled coil, a four-helix bundle, a peptide epitope, or a PNA, or any other type of assembly unit disclosed herein.

In certain embodiments, the invention provides an initiator assembly unit which comprises at least one joining element. In other embodiments, the invention provides an initiator assembly unit with two or more joining elements.

Initiator units may be tethered to a matrix in a variety of ways. The choice of tethering method will be determined by several design factors including, but not limited to: the type of initiator unit, whether the finished nanostructure must be removed from the matrix, the chemistry of the finished nanostructure, etc. Potential tethering methods include, but are not limited to, antibody binding to initiator epitopes, His tagged initiators, initiator units containing matrix binding domains (e.g., chitin-binding domain, cellulose-binding domain), antibody binding proteins (e.g., protein A or protein G) for antibody or antibody-derived initiator units, streptavidin binding of biotinylated initiators, PNA tethers, and specific covalent attachment of initiators to matrix.

In certain embodiments, an initiator unit is immobilized on a solid substrate. Initiator units may be immobilized on solid substrates using methods commonly used in the art for immobilization of antibodies or antigens. There are numerous methods well known in the art for immobilization of antibodies or antigens. These methods include non-specific adsorption onto plastic ELISA plates; biotinylation of a protein, followed by immobilization by binding onto streptavidin or avidin that has been previously adsorbed to a plastic substrate (see, e.g., Sparks et al., 1996, Screening phage-displayed random peptide libraries, in Phage Display of Peptides and Proteins, A Laboratory manual, editors, B. K. Kay, J. Winter and J. McCafferty, Academic Press, San Diego, pp. 227-53). In addition to ELISA microtiter plates, protein may be immobilized onto any number of other solid supports such as Sepharose (Dedman et al., 1993, Selection of target biological modifiers from a bacteriophage library of random peptides: the identification of novel calmodulin regulatory peptides, J. Biol. Chem. 268; 23025-30) or paramagnetic beads (Sparks et al., 1996, Screening phage-displayed random peptide libraries, in Phage Display of Peptides and Proteins, A Laboratory manual, editors, B. K. Kay, J. Winter and J. McCafferty, Academic Press, San Diego, pp. 227-53). Additional methods that may be used include immobilization by reductive amination of amine-containing biological molecules onto aldehyde-containing solid supports (Hermanson, 1996, Bioconjugate Techniques, Academic Press, San Diego, p. 186), and the use of dimethyl pimelimidate (DMP), a homobifunctional cross-linking agent that has imidoester groups on either end (Hermanson, 1996, Bioconjugate Techniques, Academic Press, San Diego, pp. 205-06). This reagent has found use in the immobilization of antibody molecules to insoluble supports containing bound protein A (e.g., Schneider et al., 1982, A one-step purification of membrane proteins using a high efficiency immunomatrix, J. Biol. Chem. 257, 10766-69).

In a specific embodiment, an initiator unit is a diabody that comprises a tethering domain (T) that recognizes and binds an immobilized antigen/hapten and an opposing domain (A) to which additional assembly units are sequentially added in a staged assembly. Antibody 8F5, which is directed against the antigenic peptide VKAETRLNPDLQPTE (SEQ ID NO: 159) derived human rhinovirus (Serotype 2) viral capsid protein Vp2, is used as the T domain (Tormo et al., 1994, Crystal structure of a human rhinovirus neutralizing antibody complexed with a peptide derived from viral capsid protein VP2, EMBO J. 13(10): 2247-56). The A domain is the same lysozyme anti-idiotopic antibody (E5.2) previously described for Diabody Unit 1. The completed initiator assembly unit therefore contains 8F5×730.1.4 (T×A) as the opposing CDRs. The initiator unit is constructed and functionally characterized using the methods described herein for characterizing joining elements and/or structural elements comprising diabodies.

In order to immobilize the initiator unit onto a solid support matrix, the rhinovirus antigenic peptide may fused to the protease recognition peptide factor Xa through a short flexible linker spliced at the N termini of the Factor Xa sequence, IEGR, (Nagai and Thogersen, 1984, Generation of beta-globin by sequence-specific proteolysis of a hybrid protein produced in Escherichia coli, Nature 309(5971): 810-12) and between the Factor Xa sequence and the antigenic peptide sequence. This fusion peptide may be covalently linked to CH-Sepharose 4B (Pharmacia); a sepharose derivative that has a six-carbon long spacer arm and permits coupling via primary amines. (Alternatively, Sepharose derivatives for covalent attachment via carboxyl groups may be used.) The covalently attached fusion protein will serve as a recognition epitope for the tethering domain “8F5” in the initiator unit (T×A).

Once the initiator is immobilized, additional diabody units (diabody assembly units 1 and 2) may be sequentially added in a staged assembly, unidirectionally from binding domain A′. Upon completion of the staged assembly, the nanostructure may be either cross-linked to the support matrix or released from the matrix upon addition of the protease Factor Xa. The protease will cleave the covalently attached antigenic/Factor Xa fusion peptide, releasing the intact nanostructure from the support matrix, since, by design, there are no Factor Xa recognition sites contained within any of the designed protein assembly units.

An alternate strategy of cleaving the peptide fusion from the solid support matrix that does not require the addition of Factor Xa, can also be implemented. This method utilizes a cleavable spacer arm attached to the sepharose matrix. The antigen peptide is covalently attached through a phenyl-ester linkage to the matrix. Once the immobilized antibody binds initiator assembly unit, the initiator assembly unit remains tethered to the support matrix until chemical cleavage of the spacer arm with imidazoleglycine buffer at pH 7.4 at which point the initiator unit/antigen complex (and associated nanostructure) are released from the support matrix.

Methods for Characterizing Joining Elements

Methods for Identifying Joining-Element Interactions by Antibody-Phage-Display Technology

In certain embodiments of the invention, joining elements suitable for use in the methods of the invention are screened and their interactions identified using antibody-phage-display technology. Phage-display technology for production of recombinant antibodies, or binding derivatives or binding fragments thereof, can be used to produce proteins capable of binding to a broad range of diverse antigens, both organic and inorganic (e.g. proteins, peptides, nucleic acids, sugars, and semiconducting surfaces, etc.). Methods for phage-display technology are well known in the art (see, e.g., Marks et al., 1991, By-passing immunization: human antibodies from V-gene libraries displayed on phage, J. Mol. Biol. 222: 581-97; Nissim et al., 1994, Antibody fragments from a “single pot” phage display library as immunochemical reagents, EMBO J. 13: 692-98; De Wildt et al., 1996, Characterization of human variable domain antibody fragments against the U1 RNA-associated A protein, selected from a synthetic and patient derived combinatorial V gene library, Eur. J. Immunol. 26: 629-39; De Wildt et al., 1997, A new method for analysis and production of monoclonal antibody fragments originating from single human B-cells, J. Immunol. Methods. 207: 61-67; Willems et al., 1998, Specific detection of myeloma plasma cells using anti-idiotypic single chain antibody fragments selected from a phage display library, Leukemia 12: 1295-1302; van Kuppevelt et al., 1998, Generation and application of type-specific anti-heparin sulfate antibodies using phage display technology, further evidence for heparin sulfate heterogeneity in the kidney, J. Biol. Chem. 273: 12960-66; Hoet et al., 1998, Human monoclonal autoantibody fragments from combinatorial antibody libraries directed to the U1snRNP associated U1C protein, epitope mapping, immunolocalization and V-gene usage, Mol. Immunol. 35: 1045-55).

Whereas recombinant antibody technology permits the isolation of antibodies with known specificity from hybridoma cells, it does not allow for the rapid creation of specific mAbs. Separate immunizations, followed by cell fusions to generate hybridomas are required to generate each mAb of interest. This can be time consuming as well as laborious.

In preferred embodiments, antibody-phage-display technology is used to overcome these limitations, so that mAbs that recognize particular antigens of interest can be generated more effectively (for methods, see Winter et al, 1994, Making antibodies by phage display technology, Ann. Rev. Immunol. 12: 433-55; Hayashi et al., 1995, A single expression system for the display, purification and conjugation of single-chain antibodies, Gene 160(1): 129-30; McGuinness et al, 1996, Phage diabody repertoires for selection of large numbers of bispecific antibody fragments, Nat. Biotechnol. 14(9): 1149-54; Jung et al., 1999, Selection for improved protein stability by phage display, J. Mol. Biol. 294(1): 163-80; Viti et al., 2000, Design and use of phage display libraries for the selection of antibodies and enzymes, Methods Enzymol. 326: 480-505). Generally, in antibody-phage-display technology, the Fv or Fab antigen-binding portions of V_{L} and the V_{H} genes are “rescued” by PCR amplification using the appropriate primers, from cDNA derived from human spleen or human peripheral blood lymphocyte cells. The rescued V_{L} and the V_{H} gene repertoires (DNA sequences) are spliced together and inserted into the minor coat protein of a bacteriophage (e.g., M13 or fd, or a binding derivative thereof) to create a fusion bacteriophage coat protein (Chang et al., 1991, Expression of antibody Fab domains on bacteriophage surfaces. Potential use for antibody selection, J. Immunol. 147(10): 3610-14; Kipriyanov and Little, 1999, Generation of recombinant antibodies, Mol. Biotechnol. 12(2): 173-201). The resulting bacteriophage contain a functional antibody fused to the outer surface of the phage protein coat and a copy of the gene fragment encoding the antibody V_{L} and V_{H} incorporated into the phage genome.

Using these methods, bacteriophage displaying antibodies that have affinity towards a particular antigen of interest can be isolated by, e.g., affinity chromatography, via the binding of a population of recombinant bacteriophage carrying the displayed antibody to a target epitope or antigen, which is immobilized on a solid surface or matrix. Repeated cycles of binding, removal of unbound or weakly-bound phage particles, and phage replication yield an enriched population of bacteriophage carrying the desired V_{L} and V_{H} gene fragments.

Antigens of interest may include peptides, proteins, immunoglobulin constant regions, CDRs (for production of anti-idiotypic antibodies) other macromolecules, haptens, small molecules, inorganic particles and surfaces.

Once purified, the linked V_{L} and V_{H} gene fragments can be rescued from the bacteriophage genome by standard DNA molecular techniques known in the art, cloned and expressed. The number of antibodies created by this method is directly correlated to the size and diversity of the gene repertoire and offers an optimal method by which to create diverse antibody libraries that can be screened for antigenicity towards virtually any target molecule. mAbs that have been created by antibody-phage-display technology often demonstrate specific binding towards antigen in the picomolar to nanomolar range (Sheets et al., 1998, Efficient construction of a large nonimmune phage antibody library: the production of high-affinity human single-chain antibodies to protein antigens, Proc. Natl. Acad. Sci. USA 95(11): 6157-62).

Antibodies, or binding derivatives or binding fragments thereof, that are useful in the methods of the invention may be selected using an antibody or fragment phage display library constructed and characterized as described above. Such an approach has the advantage of providing methods for efficiently screening a library having a high complexity (e.g. 10⁹), so as to dramatically increase identification of antibodies or fragments suitable for use in the methods of the invention.

In certain embodiments, methods for cloning an immunoglobulin repertoire (“repertoire cloning”) are used to produce an antibody for use in the staged-assembly methods of the invention. Repertoire cloning may be used for the production of virtually any kind of antibody without involving an antibody-producing animal. Methods for cloning an immunoglobulin repertoire (“repertoire cloning”) are well known in the art, and can be performed entirely in vitro. In general, to perform repertoire cloning, messenger RNA (mRNA) is extracted from B lymphocytes obtained from peripheral blood. The mRNA serves as a template for cDNA synthesis using reverse transcriptase and standard protocols (see, e.g., Clinical Gene Analysis and Manipulation, Tools, Techniques and Troubleshooting, Sections IA, IC, IIA, IIB, IIC and IIIA, Editors Janusz A. Z. Jankowski, Julia M. Polak, Cambridge University Press 2001; Sambrook et al., 2001, Molecular Cloning, A Laboratory Manual, Third Edition, Chapters 7, 11, 14 and 18, Cold Spring Harbor Laboratory Press, N.Y.; Ausubel et al., 1989, Current Protocols in Molecular Biology, Chapters 3, 4, 11, 15 and 24, Green Publishing Associates and Wiley Interscience, N.Y.). Immunoglobulin cDNAs are specifically amplified by PCR, using the appropriate primers, from this complex mixture of cDNA. In order to construct immunoglobulin fragments with the desired binding properties, PCR products from genes encoding antibody light (L) and heavy (H) chains are obtained. The products are then introduced into a phagemid vector. Cloned genes or gene fragments incorporated into the bacteriophage genome as fusions with a phage coat protein, are expressed in a suitable bacterial host leading to the synthesis of a hybrid scFv immunoglobulin molecule that is carried on the surface of the bacteriophage. Therefore the bacteriophage population represents a mixture of immunoglobulins with all specificities included in the repertoire.

Antigen-specific immunoglobulin is selected from this population by an iterative process of antigen immunoadsorption followed by phage multiplication. A bacteriophage specific only for an antigen of interest will remain following multiple rounds of selection, and may be introduced into a new vector and/or host for further engineering or to express the phage-encoded protein in soluble form and in large amounts.

Antibody phage display libraries can thus be used, as described above, for the isolation, refinement, and improvement of epitope-binding regions of antibodies that can be used as joining elements in the construction of assembly units for use in the staged assembly of nanostructures, as disclosed herein.

Methods for Characterizing Joining-Element Interactions Using X-ray Crystallography

In many instances, molecular recognition between proteins or between proteins and peptides may be determined experimentally. In one aspect of the invention, the protein-protein interactions that define the joining element interactions, and are critical for formation of a joining pair are characterized and identified by X-ray crystallographic methods commonly known in the art. Such characterization enables the skilled artisan to recognize joining pair interactions that may be useful in the compositions and methods of the present invention.

Methods for Characterizing Joining-Element Specificity and Affinity

Verification that two complementary joining elements interact with specificity may be established using, for example, ELISA assays, analytical ultracentrifugation, or BIAcore methodologies (Abraham et al., 1996, Determination of binding constants of diabodies directed against prostate-specific antigen using electrochemiluminescence-based immunoassays, J. Mol. Recognit. 9(5-6): 456-61; Atwell et al., 1996, Design and expression of a stable bispecific scFv dimer with affinity for both glycophorin and N9 neuraminidase, Mol. Immunol. 33(17-18): 1301-12; Muller et al. 1998), A dimeric bispecific miniantibody combines two specificities with avidity, FEBS Lett. 432(1-2): 45-49), or other analogous methods well known in the art, that are suitable for demonstrating and/or quantitating the strength of intermolecular binding interactions.

Design and Engineering of Structural, Joining and Functional Elements

Design of structural, joining and functional elements of the invention, and of the assembly units that comprise them, is facilitated by analysis and determination of those structures in the desired binding interaction, as revealed in a defined crystal structure, or through homology modeling based on a known crystal structure of a highly homologous material. Design of a useful assembly unit comprising one or more functional elements preferably involves a series of decisions and analyses that may include, but are not limited to, some or all of the following steps:

(i) selection of the functional elements to be incorporated based on the desired overall function of the nanostructure;

(ii) selection of the desired geometry based on the target function, in particular, determination of the relative positions of the functional elements;

(iii) selection of joining elements through determination, identification or selection of those peptides or proteins, e.g. from a combinatorial library, that have specificity for the functional nanoparticles to be incorporated into the desired nanostructure;

(iv) based on the needed separations between functional elements comprising, e.g. nanoparticles such as quantum dots, etc., selection of structural elements that will provide a suitably rigid structure with correct dimensions and having positions for incorporation of joining elements with the correct geometry and stoichiometry;

(v) design of fusion proteins incorporating peptide or protein joining elements, from step (iii) and the structural element selected in step (iv) such that the folding of the structural and joining elements of the assembly unit are not disrupted (e.g., through incorporation at β-turns);

(vi) computer modeling of the resultant fusion proteins in the context of the overall design of the nanostructure and refining of the design to optimize the structural dimensions as required by the functional specifications; or

(vii) design of the assembly sequence for staged assembly.

Modification of a structural element protein, for example, usually involves insertion, deletion, or modification of the amino acid sequence of the protein in question. In many instances, modifications involve insertions or substitutions to add joining elements not extant in the native protein. A non-limiting example of a routine test to determine the success of an insertion mutation is a circular dichroism (CD) spectrum. The CD spectrum of the resultant fusion mutant protein can be compared to the CD of the native protein.

If the insert is small (e.g., a short peptide), then the spectra of a properly folded insertion mutant will be very similar to the spectra of the native protein. If the insertion is an entire protein domain (e.g. single chain variable domain), then the CD spectrum of the fusion protein should correspond to the sum of the CD spectra of the individual components (i.e. that of the native protein and fusion protein comprising the native protein and the functional element). This correspondence provides a routine test for the correct folding of the two components of the fusion protein.

Preferably, a further test of the successful engineering of a fusion protein is made. For example, an analysis may be made of the ability of the fusion protein to bind to all of its targets, and therefore, to interact successfully with all joining pairs. This may be performed using a number of appropriate ELISA assays; at least one ELISA is performed to test the affinity and specificity of the modified protein for each of the joining pairs required to form the nanostructure.

Uses of the Staged-Assembly Method and of Nanostructures Constructed Thereby

The staged-assembly methods and the assembly units of the invention have use in the construction of myriad nanostructures. The uses of such nanostructures are readily apparent and include applications that require highly regular, well-defined arrays of one-, two-, and three-dimensional structures such as fibers, cages, or solids, which may include specific attachment sites that allow them to associate with other materials.

In certain embodiments, the nanostructures fabricated by the staged assembly methods of the invention are one-dimensional structures. For example, nanostructures fabricated by staged assembly can be used for structural reinforcement of other materials, e.g., aerogels, paper, plastics, cement, etc. In certain embodiments, nanostructures that are fabricated by staged assembly to take the form of long, one-dimensional fibers are incorporated, for example, into paper, cement or plastic during manufacture to provide added wet and dry tensile strength.

In another embodiment, the nanostructure is a patterned or marked fiber that can be used for identification or recognition purposes. In such embodiments, the nanostructure may contain such functional elements as e.g., a fluorescent dye, a quantum dot, or an enzyme.

In a further embodiment, a particular nanostructure is impregnated into paper and fabric as an anti-counterfeiting marker. In this case, a simple color-linked antibody reaction (such as those commercially available in kits) is used to verify the origin of the material. Alternatively, such a nanostructure could bind dyes, inks or other substances, either before or after incorporation, to color the paper or fabrics or to modify their appearance or properties in other ways.

In another embodiment, nanostructures are incorporated, for example, into ink or dyes during manufacture to increase solubility or miscibility.

In another embodiment, a one-dimensional nanostructure e.g., a fiber, bears one or more enzyme or catalyst functional elements in desired positions. The nanostructure serves as a support structure or scaffold for an enzymatic or catalytic reaction to increase its efficiency. In such an embodiment, the nanostructure may be used to “mount” or position enzymes or other catalysts in a desired reaction order to provide a reaction “assembly line.”

In another embodiment, a one-dimensional nanostructure, e.g., a fiber, is used as an assembly jig. Two or more components, e.g., functional units, are bound to the nanostructure, thereby providing spatial orientation. The components are joined or fused, and then the resultant fused product is released from the nanostructure.

In another embodiment, a nanostructure is a one-, two- or three-dimensional structure that is used as a support or framework for mounting nanoparticles (e.g., metallic or other particles with thermal, electronic or magnetic properties) with defined spacing, and is used to construct a nanowire or nanocircuit.

In another embodiment, the staged assembly methods of the invention are used to accomplish electrode-less plating of a one-dimensional nanostructure (fiber) with metal to construct a nanowire with a defined size and/or shape. For example, a nanostructure could be constructed that comprises metallic particles as functional elements.

In another embodiment, a one-dimensional nanostructure (e.g., a fiber) comprising magnetic particles as functional elements is aligned by an external magnetic field to control fluid flow past the nanostructure. In another embodiment, the external magnetic field is used to align or dealign a nanostructure (e.g., fiber) comprising optical moieties as functional elements for use in LCD-type displays.

In another embodiment, a nanostructure is used as a size standard or marker of precise dimensions for electron microscopy.

In other embodiments, the nanostructures fabricated by the staged assembly methods of the invention are two- or three-dimensional structures. For example, in one embodiment, the nanostructure is a mesh with defined pore size and can serve as a two-dimensional sieve or filter.

In another embodiment, the nanostructure is a three-dimensional hexagonal array of assembly units that is employed as a molecular sieve or filter, providing regular vertical pores of precise diameter for selective separation of particles by size. Such filters can be used for sterilization of solutions (i.e., to remove microorganisms or viruses), or as a series of molecular-weight cut-off filters. In this embodiment, the protein components of the pores, such as structural elements or functional elements, may be modified so as to provide specific surface properties (i.e., hydrophilicity or hydrophobicity, ability to bind specific ligands, etc.). Among the advantages of this type of filtration device is the uniformity and linearity of pores and the high pore to matrix ratio.

It will be apparent to one skilled in the art that the methods and assembly units disclosed herein may be used to construct a variety of two- and three-dimensional structures such as polygonal structures (e.g., octagons), as well as open solids such as tetrahedrons, icosahedrons formed from triangles, and boxes or cubes formed from squares and rectangles (e.g., the cube disclosed in Section 11, Example 6). The range of structures is limited only by the types of joining and functional elements that can be engineered on the different axes of the structural elements.

In another embodiment, a two- or three-dimensional nanostructure may be used to construct a surface coating comprising optical, electric, magnetic, catalytic, or enzymatic moieties as functional units. Such a coating could be used, for example, as an optical coating. Such an optical coating could be used to alter the absorptive or reflective properties of the material coated.

A surface coating constructed using nanostructures of the invention could also be used as an electrical coating, e.g., as a static shielding or a self-dusting surfaces for a lens (if the coating were optically clear). It could also be used as a magnetic coating, such as the coating on the surface of a computer hard drive.

Such a surface coating could also be used as a catalytic or enzymatic coating, for example, as surface protection. In a specific embodiment, the coating is an antioxidant coating.

In another embodiment, the nanostructure may be used to construct an open framework or scaffold with optical, electric, magnetic, catalytic, enzymatic moieties as functional elements. Such a scaffold may be used as a support for optical, electric, magnetic, catalytic, or enzymatic moieties as described above. In certain embodiments, such a scaffold could comprise functional elements that are arrayed to form thicker or denser coatings of molecules, or to support soluble micron-sized particles with desired optical, electric, magnetic, catalytic, or enzymatic properties.

In another embodiments, a nanostructure serves as a framework or scaffold upon which enzymatic or antibody binding domains could be linked to provide high density multivalent processing sites to link to and solubilize otherwise insoluble enzymes, or to entrap, protect and deliver a variety of molecular species.

In another embodiment, the nanostructure may be used to construct a high density computer memory with addressable locations.

In another embodiment, the nanostructure may be used to construct an artificial zeolite, i.e., a natural mineral (hydrous silicate) that has the capacity to absorb ions from water, wherein the design of the nanostructure promotes high efficiency processing with reactant flow-through an open framework.

In another embodiment, the nanostructure may be used to construct an open framework or scaffold that serves as the basis for a new material, e.g., the framework may possess a unique congruency of properties such as strength, density, determinate particle packing and/or stability in various environments.

In certain embodiments, the staged-assembly methods of the invention can also be used for constructing computational architectures, such as quantum cellular automata (QCA) that are composed of spatially organized arrays of quantum dots. In QCA technology, the logic states are encoded by positions of individual electrons, contained in QCA cells composed of spatially positioned quantum dots, rather than by voltage levels. Staged assembly can be implemented in an order that spatially organizes quantum dot particles in accordance with the geometries necessary for the storage of binary information. Examples of logic devices that can be fabricated using staged assembly for the spatially positioning and construction of QCA cells for quantum dot cellular automata include QCA wires, QCA inverters, majority gates and full adders (Amlani et al., 1999, Digital logic gate using quantum-dot cellular automata, Science 284(5412): 289-91; Cowburn and Welland, 2000), Room temperature magnetic quantum cellular automata, Science 287(5457): 1466-68; Orlov et al, 1997, Realization of a Functional Cell for Quantum-Dot Automata, Science 277: 928-32).

The invention will now be further described with reference to the following, non-limiting examples.

### EXAMPLE 1

### Protocol for Staged Assembly

The following steps of staged assembly are illustrated in FIG. 8. The resultant nanostructure is illustrated FIG. 8, Step 11.

| Staged Assembly Steps | Procedure |
|---|---|
| Step 1 | a) Add assembly unit-1 |
| | b) Wash |
| Step 2 | a) Add assembly unit-2 |
| | b) Wash |
| Step 3 | a) Repeat Step 1 |
| Step 4 | a) Add assembly unit-3 |
| | b) Wash |
| Step 5 | a) Repeat Step 1 |
| Step 6 | a) Add assembly unit-4 |
| | b) Wash |
| Step 7 | a) Repeat Step 2 |
| Step 8 | a) Add assembly unit-5 |
| | b) Wash |
| Step 9 | a) Repeat Step 1 |
| Step 10 | a) Repeat Step 2 |
| Step 11 | a) Repeat Step 1 |

### EXAMPLE 2

### Fabrication of a Macromolecular Nanostructure

To build a macromolecular assembly, two assembled nanostructures intermediates can be joined to one another using the staged assembly methods of the invention. This example describes the fabrication of a macromolecular nanostructure from two nanostructure intermediates.

FIG. 9 illustrates the staged assembly of the two nanostructure intermediates fabricated from the staged assembly protocol illustrated in FIG. 8. Nanostructure intermediate-1 is illustrated as Step-11 in FIG. 8. Nanostructure intermediate-2 is illustrated as Step-8 in FIG. 8. The following protocol describes the addition of two nanostructure intermediates by the association of a complementary joining pair as illustrated in FIG. 9. The resultant macromolecular nanostructure is illustrated FIG. 9, Step 5.

| Staged Assembly Steps | Procedure |
|---|---|
| Step 1 | Steps 1-11 of staged assembly protocol |
| | described above in Section 8 (Example 3) |
| Step 2 | a) Add A′ capping unit |
| | b) Wash |
| Step 3 | Remove nanostructure intermediate-1 from |
| | the support matrix and isolate |
| Step 4 | Perform Steps 1-8 of staged assembly |
| | protocol described above in Section 8 |
| | (Example 3), leaving nanostructure |
| | intermediate-2 attached to the support |
| | matrix |
| Step 5 | a) Add nanostructure intermediate-1 |
| | b) Wash |

### EXAMPLE 3

### Analysis of Polymerization by Light Scattering

The extent polymerization of macromolecular monomers, such as the diabodies used in this example, may be analyzed by light scattering. Light scattering measurements from a light scattering photometer, e.g., the DAWN-DSP photometer (Wyatt Technology Corp., Santa Barbara, Calif.), provides information for determination of the weight average molecular weight, determination of particle size, shape and particle-particle pair correlations.

### EXAMPLE 4

### Molecular Weight Determination (Degree of Polymerization) by Sucrose Gradient Sedimentation

Linked diabody units of different lengths sediment at different rates in a sucrose gradient in zonal ultracentrifugation. The quantitative relationship between the degree of polymerization and sedimentation in Svedberg units is then calculated. This method is useful for characterizing the efficiency of self-assembly in general, as well as the process of staged assembly at each step of addition of a new diabody unit.

### EXAMPLE 5

### Morphology and Length of Rods by Electron Microscopy

After sucrose gradient fractionation and SDS-PAGE analysis, the partially purified fractions containing rods are apparent. Samples of the appropriate fractions are placed on EM grids and stained or shadowed to look for large structures using electron microscopy in order to determine their morphology.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A method for staged assembly of a nanostructure comprising:
(a) contacting a nanostructure intermediate comprising at least one unbound joining element with an assembly unit comprising a plurality of different joining elements, wherein:
(i) none of the joining elements of said plurality of different joining elements can interact with itself or with another joining element of said plurality, and
(ii) a single joining element of said plurality and a single unbound joining element of the nanostructure intermediate are complementary joining element,
whereby the assembly unit is non-covalently bound to the nanostructure intermediate to form a new nanostructure intermediate for use in subsequent cycles;
(b) removing unbound assembly units; and
(c) repeating steps (a) and (b) for a sufficient number of cycles to form a nanostructure,
wherein the assembly unit in at least one cycle comprises a peptide nucleic acid.

2. The method of claim 1, wherein the nanostructure intermediate comprises a surface-bound initiator assembly unit.

3. The method of claim 1, comprising the additional step of:
(d) capping the nanostructure with at least one capping unit.

4. The method of claim 1, wherein a first assembly unit used in at least one cycle comprises at least one structural element covalently linked to a first joining element comprising a peptide nucleic acid.

5. The method of claim 4, wherein the structural element is covalently linked to the first joining element and to a second joining element.

6. The method of claim 5, wherein the second joining element comprises a peptide nucleic acid.

7. The method of claim 4, wherein the first assembly unit comprises a first structural element that is bound to a second structural element to form a stable complex.

8. The method of claim 4, wherein the assembly unit further comprises a functional element.

9. The method of claim 8, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

10. The method of claim 8, wherein the functional element comprises a peptide nucleic acid.

11. The method of claim 1, wherein a first assembly unit used in at least one cycle comprises a functional element and a joining element comprising a peptide nucleic acid.

12. The method of claim 11, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

13. The method of claim 11, wherein the functional element comprises a peptide nucleic acid.

14. The method of claim 1, further comprising the step of post-assembly conversion of specific non-covalent interactions of complementary joining elements to covalent linkages, whereby the linkages are stabilized.

15. The method of claim 1, wherein the assembly unit comprises a plurality of sub-assembly units that bind to each other to form a stable complex.

16. A nanostructure formed from a plurality of species of assembly units comprising a plurality of different joining elements, said assembly units including at first assembly unit comprising a peptide nucleic acid.

17. The nanostructure of claim 16, wherein the peptide nucleic acid in the first assembly unit is present as a joining element.

18. The nanostructure of claim 17, wherein the first assembly unit further comprises a functional element.

19. The nanostructure of claim 18, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

20. The nanostructure of claim 18, wherein the functional element comprises a peptide nucleic acid.

21. The nanostructure of claim 17, wherein the peptide nucleic acid in the first assembly unit is present as a functional element.
